SEQUENCE LISTING (4 sequences)

SEQ ID NO: 1 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 2 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 3 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 4 - protein, 6 aa, Homo sapiens
AVEAGF

(19) United States
(12) Patent Application Publication
(10) Pub. No.: US 2012/0010230 A1
(43) Pub. Date: Jan. 12, 2012

(54) METHODS AND COMPOSITIONS FOR IDENTIFICATION, ASSESSMENT AND TREATMENT OF CANCERS ASSOCIATED WITH HEDGEHOG SIGNALING

(76) Inventors: John R. MacDougall, Hingham, MA (US); Juan Guillermo Paez, Spencer, MA (US); Karen J. McGovern, Groton, MA (US); Jeffery L. Kutok, Natick, MA (US); Georgios Skliris, Cambridge, MA (US); Kerry White, Medford, MA (US); Kerrie L. Faia, Topsfield, MA (US); Marisa Osswalt Peluso, Brookline, MA (US); Veronica Travaglione Campbell, Framingham, MA (US)

(21) Appl. No.: 13/179,438

(22) Filed: Jul. 8, 2011

Related U.S. Application Data
(60) Provisional application No. 61/362,541, filed on Jul. 8, 2010.
(60) Provisional application No. 61/362,557, filed on Jul. 8, 2010.
(60) Provisional application No. 61/471,085, filed on Apr. 1, 2011.
(60) Provisional application No. 61/492,594, filed on Jun. 2, 2011.

(51) Int. Cl. A61K 31/4355; A61P 35/02; G01N 33/53; C12Q 1/02; C12Q 1/68; C12Q 1/37; C12Q 1/48; A61P 35/00; C40B 30/04

(52) U.S. Cl. 514/278; 435/7.1; 506/9; 435/6.12; 435/23; 435/15; 435/29

(57) ABSTRACT

Provided herein are methods, assays and kits for evaluating a sample, e.g., a sample obtained from a cancer patient, to detect one or more hedgehog biomarkers and/or one or more cilium markers. Thus, the invention can be used, inter alia, as a means to identify patients likely to benefit from administration of one or more hedgehog inhibitors, alone or in combination with therapeutic agents; to predict a time course of disease or a probability of a significant event in the disease of a cancer patient; to stratify patient populations; and/or to more effectively treat or prevent a cancer or a tumor associated with hedgehog signaling.

### CROSS REFERENCE TO RELATED APPLICATIONS

This application claims the benefit of priority to U.S. Provisional application Ser. No. 61/362,541, filed Jul. 8, 2010; U.S. Provisional application Ser. No. 61/362,557, filed Jul. 8, 2010; U.S. Provisional application Ser. No. 61/471,085, filed Apr. 1, 2011; and U.S. Provisional application Ser. No. 61/492,594, filed Jun. 2, 2011. The contents of all of the aforesaid applications are hereby incorporated by reference in their entirety. A PCT patent application entitled “Methods and Compositions for Identification, Assessment and Treatment of Cancers Associated with Hedgehog Signalling,” filed Jul. 8, 2011 with the U.S. Receiving Office and designating attorney docket number I2041-7003WO is also incorporated by reference.

### SEQUENCE LISTING

The instant application contains a Sequence Listing which has been submitted in ASCII format via EFS-Web and is hereby incorporated by reference in its entirety. Said ASCII copy, created on Jul. 7, 2011, is named 124122PC.txt and is 11,563 bytes in size.

### BACKGROUND

Hedgehog signaling plays a role in many stages of development, especially in the formation of left-right symmetry. Loss or reduction of hedgehog signaling leads to multiple developmental deficits and malformations, one of the most striking of which is cyclopia.

Many cancers and proliferative conditions have been shown to depend on the hedgehog pathway. It has been reported that activating hedgehog pathway mutations occur in sporadic basal cell carcinoma (Xie et al. (1998) Nature 391: 90-2) and primitive neuroectodermal tumors of the central nervous system (Reifenberger et al. (1998) Cancer Res 58: 1798-803). Uncontrolled activation of the hedgehog pathway has also been shown in other numerous cancer types, such as cancers of the gastrointestinal tract including pancreatic, esophageal, and gastric cancer (Berman et al. (2003) Nature 425: 846-51, Thayer et al. (2003) Nature 425: 851-56); lung cancer (Watkins et al. (2003) Nature 422: 313-317); prostate cancer (Karhadkar et al. (2004) Nature 431: 707-12, Sheng et al. (2004) Molecular Cancer 3: 29-42, Fan et al. (2004) Endocrinology 145: 3961-70); breast cancer (Kubo et al. (2004) Cancer Research 64: 6071-74, Lewis et al. (2004) Journal of Mammary Gland Biology and Neoplasia 2: 165-181); hepatocellular cancer (Sicklick et al. (2005) ASCO conference, Mohini et al. (2005) AACR conference); and neuroendocrine cancers (also known as gastroenteropancreatic tumors or gastroenteropancreatic neuroendocrine cancers).

There is a need to identify reliable biomarkers that are predictive of likelihood of efficacy of treatments of cancers associated with hedgehog signaling, and in particular therapies involving hedgehog inhibitors.

### SUMMARY

The present invention provides methods, assays and kits for evaluating a sample, e.g., a sample obtained from a cancer patient, to detect a hedgehog (Hh)-associated biomarker. In certain embodiments, responsiveness to a hedgehog signal (e.g., an Hh ligand or Hh inhibitor) in a cancer or tumor cell is correlated with the level of one or more Hh-associated biomarkers. In one embodiment, the hedgehog-associated biomarker evaluated is an alteration in nuclear Glioma-associated oncogene family zinc finger 1 (Gli1), e.g., a change in nuclear Gli-1 (e.g., a change in one or more of: Gli1 expression, subcellular localization or translocation of Gli1 into the nucleus, or a change in Gli1 stability). Alternatively, or in combination with nuclear Gli1, one or more of the following hedgehog biomarkers can be evaluated: the presence or absence of primary cilia, an alteration in desmoplasia, or an alteration in a pericytic marker. For example, the presence of primary cilia, an increased nuclear Gli1, and/or an increased level in a pericytic marker, in a tumor or cancer can serve as a predictive biomarker of hedgehog responsiveness. Thus, detection of a hedgehog-associated marker can provide a useful diagnostic, predictive, and prognostic reagent for identifying a subject (e.g., a cancer patient) in need of therapy, or likely to be responsive to therapy, with a hedgehog inhibitor. Accordingly, methods, assays and kits of the invention can be used, inter alia, to identify patients likely to benefit from administration of a hedgehog inhibitor, alone or in combination with a cancer therapy (e.g., one or more therapeutic agents, radiation and/or surgery); to predict a time course of disease or a probability of a significant event in the disease of a cancer patient; to stratify patient populations; and/or to more effectively treat or prevent a cancer or a tumor associated with hedgehog signaling.

In one aspect, the invention features a method of, or assay for, evaluating a sample, e.g., a cancer or tumor sample (e.g., a sample from a cancer patient). The method or assay includes: detecting a hedgehog-associated biomarker (e.g., an alteration in a hedgehog-associated biomarker chosen from one or more of: increased nuclear Gli1, the presence or absence of primary cilia or a phosphorylated hedgehog receptor (e.g., phosphorylated Smoothened (Smo)) in the cilia, an alteration in desmoplasia, or an increased pericytic marker). The presence of an alteration in one or more hedgehog-associated biomarkers in the sample indicates an increased or decreased responsiveness of the cancer or tumor to hedgehog signaling, and/or a need for hedgehog inhibition therapy. In one embodiment, the hedgehog-associated biomarker evaluated is Gli1, e.g., nuclear Gli1. Alternatively, or in combination with nuclear Gli1, one or more of the following hedgehog biomarkers can be evaluated: the presence or absence of primary cilia or a phosphorylated hedgehog receptor (e.g., phosphorylated Smo) in the cilia, an alteration in desmoplasia, or an alteration in a pericytic marker, in the sample. In one embodiment, the presence of primary cilia or the phosphorylated hedgehog receptor (e.g., phosphorylated Smo) in the cilia, an increased nuclear Gli1, and/or an increased pericytic marker, in a tumor or cancer is indicative of increased responsiveness of the cancer or tumor to a hedgehog inhibitor. Conversely, the absence of primary cilia, decreased nuclear Gli1, and/or decreased pericytic marker, in a tumor or cancer is indicative of decreased responsiveness of the cancer or tumor to a hedgehog inhibitor.

The method, or assay, can further include one or more of the following: (i) identifying a subject (e.g., a patient, patient group or population), having a cancer or a tumor, or at risk of developing a cancer or a tumor, as having an increased or a decreased likelihood to respond to treatment with a hedgehog inhibitor; (ii) determining a treatment regimen upon evaluation of the sample (e.g., selecting or altering the course of therapy, dosing, treatment schedule or time course, and/or combination therapy); (iii) analyzing a time course of the cancer or tumor in the subject; and/or (iv) analyzing the probability of a significant event in the subject with the cancer or tumor. Detection of an alteration in the hedgehog-associated biomarker (e.g., one or more of: increased nuclear Gli1, the presence or absence of primary cilia or a phosphorylated hedgehog receptor (e.g., phosphorylated Smo) in the cilia, an alteration in desmoplasia, or an increased pericytic marker) can indicate one or more of the following: identifies the subject as having an increased or decreased likelihood to respond to treatment with the hedgehog inhibitor; determines the treatment regimen to be used; analyzes or predicts the time course of the cancer or tumor, or the probability of a significant event occurring in the subject. In one embodiment, the method or assay includes comparing the level of one or more hedgehog biomarkers to a specified parameter (e.g., a reference value or sample; a sample obtained from a healthy subject; or a non-malignant sample obtained from the subject, such as a non-malignant tissue sample or a blood matched control).

In a related aspect, the invention features a method of, or assay for, identifying a subject (e.g., a patient, a patient group or population), having a cancer or a tumor, or at risk for developing a cancer or a tumor, as having an increased or decreased likelihood to respond to a treatment with a hedgehog inhibitor. The method or assay includes: evaluating a sample from the subject, e.g., to detect a hedgehog-associated biomarker (e.g., an alteration in a hedgehog-associated biomarker chosen from one or more of: increased nuclear Gli1, the presence or absence of primary cilia or a phosphorylated hedgehog receptor (e.g., phosphorylated Smo) in the cilia, an alteration in desmoplasia, or an increased pericytic marker); and (optionally) identifying the subject having the cancer or at risk for developing the cancer as likely to respond to the treatment with the hedgehog inhibitor. The presence of an alteration in one or more hedgehog-associated biomarkers in the sample (e.g., an increased nuclear Gli1, and/or one or more of: the presence of primary cilia or the phosphorylated hedgehog receptor (e.g., phosphorylated Smo) in the cilia, an alteration in desmoplasia, or an increased level of a pericytic marker) indicates that the subject has an increased likelihood to respond to treatment with the hedgehog inhibitor (e.g., IPI-926). Conversely, the absence of primary cilia or the phosphorylated hedgehog receptor (e.g., phosphorylated Smo) in the cilia, decreased nuclear Gli1, and/or decreased pericytic marker, in a tumor or cancer is indicative of decreased responsiveness of the cancer or tumor to a hedgehog inhibitor.

In yet another aspect, the invention features a method of, or assay for, evaluating or monitoring a treatment regimen (e.g., a cancer therapy treatment regimen) in a subject (e.g., a patient, a patient group or population), having a cancer or tumor, or at risk for developing cancer or tumor. The method includes evaluating a sample from the subject, e.g., to detect a hedgehog-associated biomarker (e.g., an alteration in a hedgehog-associated biomarker chosen from one or more of: increased nuclear Gli1, the presence or absence of primary cilia or a phosphorylated hedgehog receptor (e.g., phosphorylated Smo) in the cilia, an alteration in desmoplasia, or an increased pericytic marker); and (optionally) selecting or altering one or more of the course of therapy, dosing, treatment schedule or time course, or a combination therapy (e.g., a combination of a hedgehog inhibitor with a second therapeutic agent). In one embodiment, the hedgehog-associated biomarker evaluated is Gli1, e.g., nuclear Gli1, and/or one or more of: the presence or absence of primary cilia or the phosphorylated hedgehog receptor (e.g., phosphorylated Smo) in the cilia, an alteration in desmoplasia, or an alteration in a pericytic marker, in the sample. A decrease in one or more of: Gli1, e.g., nuclear Gli1; a pericytic marker; or decrease in the cilium marker (e.g., decreased cilia or Smo phosphorylation) is indicative of improved therapeutic outcome. Conversely, an increase in one or more of: Gli1, e.g., nuclear Gli1; the pericytic marker; or in the cilium marker is indicative of reduced improvement in the therapeutic outcome. In one embodiment, the treatment regimen includes administration of one or more hedgehog inhibitors, one or more therapeutic agents, and/or radiation. The method can be used, e.g., to evaluate the suitability of, or to choose between alternative treatments, e.g., a particular dosage, mode of delivery, time of delivery, or inclusion of adjunctive therapy (e.g., administration in combination with a second therapeutic agent).

In yet another aspect, the invention features a method of, or assay for, evaluating a time course of a cancer, and/or the probability of a significant event, in a subject (e.g., a patient, a patient group or population), having a cancer or a tumor, or at risk for developing a cancer or a tumor. The method includes evaluating a sample from the subject, e.g., to detect a hedgehog-associated biomarker (e.g., an alteration in a hedgehog-associated biomarker chosen from one or more of: increased nuclear Gli1, the presence or absence of primary cilia or a phosphorylated hedgehog receptor (e.g., phosphorylated Smo) in the cilia, an alteration in desmoplasia, or an increased pericytic marker); and (optionally) comparing the detected alteration to a specified parameter (e.g., a reference value or sample). In one embodiment, the hedgehog-associated biomarker evaluated is chosen from one or more of: Gli1, e.g., nuclear Gli1; the presence or absence of primary cilia or a phosphorylated hedgehog receptor (e.g., phosphorylated Smo) in the cilia; an alteration in desmoplasia; or an alteration in a pericytic marker, in the sample, wherein a decrease in one or more of: Gli1, e.g., nuclear Gli1; the pericytic marker; or decrease in the cilium marker, is indicative of improved therapeutic outcome. Conversely, an increase in one or more of: Gli1, e.g., nuclear Gli1; the pericytic marker; or the cilium marker, is indicative of reduced improvement in the therapeutic outcome.

In certain embodiments of the methods or assays disclosed herein, the hedgehog-associated biomarker is Gli1, e.g., nuclear Gli1. In embodiments, a Gli1 gene or gene product is detected, e.g., a Gli1 mRNA or protein. The Gli1 mRNA or protein can be detected by techniques known in the art, e.g., immunostaining, immunohistochemistry, and immunofluorescence. In one embodiment, a change in the level of nuclear Gli1 is detected, e.g., an increase in nuclear Gli1 mRNA or protein. A change in the level of nuclear Gli-1 can include one or more of: a change in Gli1 expression, subcellular localization or translocation into the nucleus, or a change in Gli1 stability. In one embodiment, Gli1, e.g., nuclear Gli1, is detected in the tumor cells in the sample, e.g., in the tumor cells of a hedgehog-independent or dependent tumor sample (e.g., a BCC, medulloblastoma or sarcoma). In other embodiments, Gli1, e.g., nuclear Gli1, is detected in the stromal tissue surrounding the tumor cells in the sample (e.g., in a desmoplastic tumor or cancer such as a pancreatic cancer).

For any of the methods or assays disclosed herein, the sample can be analyzed at any stage of treatment, e.g., prior to or after administration of the hedgehog inhibitor and/or therapeutic agent, to thereby determine appropriate dosage(s) and treatment regimen(s) of the hedgehog inhibitor and/or therapeutic agent (e.g., amount per treatment or frequency of treatments) for prophylactic or therapeutic treatment of the subject. In certain embodiments, the methods of the invention include the step of detecting the level of one or more hedgehog biomarkers in the subject, prior to, or after, administering a hedgehog inhibitor, a chemotherapeutic agent, and/or radiation, to the subject. A change in one or more biomarkers (e.g., one or more of: increased Gli1 (e.g., increased nuclear Gli1); the presence of primary cilia (e.g., detection of cilia immunofluorescence (IF)) or an increased level of a pericytic marker, in the sample indicates that the tumor from which the sample was obtained is likely to therapy with a hedgehog inhibitor, alone or in combination with other therapeutic agents, surgical and/or radiation procedures.

In certain embodiments, the methods or assays disclosed herein include the step of comparing the level of one or more hedgehog biomarkers to a specified parameter (e.g., a reference value or sample; a sample obtained from a healthy subject; or a non-malignant sample obtained from the subject, such as a non-malignant tissue sample or a blood matched control).

In other embodiments, the method further includes treating a cancer or tumor harboring altered levels of the one or more hedgehog biomarkers, with one or more hedgehog inhibitors, alone or in combination with other chemotherapeutic agents, surgery and/or radiation.

In certain embodiments, the hedgehog-associated biomarkers include, but are not limited to, cytogenetic abnormalities, point mutations, deletions, changes in gene copy number, and changes in expression of a gene or gene product. In certain embodiments, the level, expression, subcellular localization, structure (e.g., post-translational modifications, such as phosphorylation) and/or activity of one or more biomarker polypeptides is evaluated. In related embodiments, the expression level, structure, and/or activity of one or more mutant isoforms, e.g., isoforms arising from one or more of alternative splicing, frameshifting, translational and/or post-translational events, of various proto-oncogene expression products in a cell, e.g., a cancerous or tumor cell, are detected. In other embodiments, the one or more alterations of the hedgehog-associated biomarkers include changes in tumor architecture, e.g., desmoplasia, tumor perfusion, interstitial fluid pressure, microvascular density, tumor-associated stroma and/or pericytes.

In other embodiments, the hedgehog-associated biomarker evaluated or treated is chosen from one or more of: an alteration in a marker of a hedgehog pathway, an alteration in a genomic marker, an alteration in a marker of Epithelial to Mesenchymal Transition (EMT), an alteration in a Gemcitabine marker, or an alteration in tumor architecture. Examples of hedgehog-associated biomarkers include, but are not limited to:

(i) an alteration in a marker of a hedgehog pathway, including but not limited to, an alteration in a gene or a gene product (e.g., DNA, RNA, protein, including alterations in sequence, activity and/or expression levels) of, a hedgehog ligand (e.g., Sonic Hedgehog (SHh), Indian Hedgehog (IHh) or Desert Hedgehog (DHh)), for example, an increase in the levels of a hedgehog ligand polypeptide, detection of a single nucleotide polymorphism of a hedgehog ligand (e.g., a SHh SNP); an alteration in a gene or a gene product (e.g., DNA, RNA, protein, including alterations in sequence, activity and/or expression levels) of, an upstream or downstream component(s) of the hedgehog signaling pathway, e.g., a hedgehog receptor (e.g., patched (PTCH) or smoothened (Smo, e.g., phosphorylated Smo)), an activator or inhibitor of hedgehog, or a signaling mediator (e.g., Gli1, Gli2, and Gli3). For example, an alterations in sequence, activity and/or expression level of any of SMO, PTCH, SUFU, GLI1 (e.g., GLI1 gene amplification or increased gene expression), GLI3, and/or BOC can be detected;

(ii) an alteration in a genomic marker, including but not limited to, an alteration in a gene or a gene product (e.g., DNA, RNA, protein, including alterations in sequence, activity and/or expression levels) of, KRAS, TGFβ-SMADs, p53, cyclin D1, or Gli1; or an oncogenic gene or gene product chosen from ALK, EGFR, PIK3CA, BRAF, PTEN, AKT, TP53, NRAS, CTNNB1 (beta-catenin), APC, KIT, JAK2, NOTCH, or FLT3;

(iii) an alteration in a marker of Epithelial to Mesenchymal Transition (EMT), including but not limited to, e.g. snail, twist, slug, vimentin, cadherins, and SPARC;

(iv) an alteration in a Gemcitabine marker, including but not limited to genes or gene products associated with metabolism, transport and DNA repair mechanisms, e.g., SLC29A1, SLC28A1, SLC28A3, CDA, NT5C, DCK, UMP/CMP kinase, RRM1, RRM2, Nucleoside diphosphate kinase, and HuR;

(v) an alteration associated with chondrosarcoma, including but not limited to, histological evaluation (e.g., evaluation of histological grade, for example, Grades I-III, wherein an increase in histological grade is associated with higher metastatic potential); cytogenetic analysis (e.g., structural aberrations of chromosomes 1, 6, 9, 12 and 15 and numerical aberrations of chromosomes 5, 7, 8 and 18); mutational analysis (e.g., somatic mutations of IDH1 and IDH2); changes in cytogenic molecular markers, such as LOH at loci (EXT, EXTL, 13q14, 17p13, 9p21 and chromosome 10); evaluation of osteogenic lesions by Magnetic Resonance Imaging (MRI) and/or CT-scan; and detection of one or ore of type II collagen (e.g., in the extracellular tumor matrix of mesenchymal chondrosarcomas), MIB-1, p53, or Ki-MCM6 (e.g., for identifying proliferative activity in Grade I chondrosarcomas); and/or

(vi) an alteration in tumor architecture, including but not limited to, alterations in tumor size; desmoplasia (e.g., evaluation of collagen, fibronectin or alpha-smooth muscle-specific actin (SMA) levels); tumor perfusion; interstitial fluid pressure; microvascular density (MVD) (e.g., evaluation of markers such as CD31 and Meca32); pericytes (e.g., evaluation of a pericytic marker, including but not limited to, NG-2 (CSPG4), RGS5, sphingosine-1-phosphate (S-1-P), PDGF-BB, N-cadherin, and PDGFR-beta) associated with a tumor or cancer; evaluation of the association of pericytes with endothelial cells; evaluation of the numbers or distribution of pericytes within a tumor or tumor-associated stroma; and/or stroma molecular markers (e.g., reactive stroma molecular signature).

In one embodiment, the hedgehog-associated biomarker is a component of hedgehog signaling (e.g., detecting selective localization of an upstream or downstream component(s) of the hedgehog signaling pathway, e.g., a hedgehog receptor (e.g., patched (PTCH) or smoothened (Smo)), an activator or inhibitor of hedgehog, or a signaling mediator (e.g., Gli1, Gli2, and Gli3)). In one embodiment, the hedgehog biomarker is a phoshorylated hedgehog receptor (e.g., phosphorylated Smo) in cilia.

In other embodiments, the hedgehog-associated biomarker is the presence or absence of one or more cilium markers, including cilium (e.g., primary cilium) or a component thereof, or the presence (or increased level) of a phosphorylated hedgehog receptor (e.g., phosphorylated Smo) in the cilia. Examples of cilia and components thereof include, but are not limited to, a microtubule or a component thereof (e.g., tubulin and rootelin), a component of intraflagellar transport (IFT), a kinesin (e.g., kinesin II), a microtubule organizing center or a component thereof, a basal body or a component thereof (e.g., basal body proteins such as CEP164, ODF2, CEP170, gamma-tubulin, rootletin, or pericentrin).

In another embodiment, the hedgehog-associated biomarker is an alteration in desmoplasia. For example, a change in the level of one or more of: collagen, fibronectin or alpha-smooth muscle-specific actin (SMA) levels. In one embodiment, an increase in one or more markers of demoplasia (e.g., increased collagen content) is indicative of increased responsiveness to an Hh inhibitor, whereas a decrease in such markers indicated an improved therapeutic outcome.

In yet another embodiment, the hedgehog-associated biomarker is a change in the level of a pericytic marker. The pericytic marker can be chosen from one or more of: NG-2 (CSPG4), RGS5, sphingosine-1-phosphate (S-1-P), PDGF-BB, N-cadherin, or PDGFR-beta. An increase in the level of a pericytic marker, such as S-1-P and/or PDGF-BB, can be indicative of increased responsiveness to a hedgehog inhibitor.

In one embodiment, the hedgehog biomarker is a hedgehog ligand, e.g., SHh, IHh or DHh.

In other embodiments, the hedgehog-associated biomarker is differentially expressed in chondrosarcoma. Non-limiting examples of such biomarkers include, but are not limited to, ADAMTSL1, BOK, C7, CES1, CNR1, DUSP10, FAM150B, FLJ38379, FRMD3, GDF10, Gli1, HGF, HhIP, ITGB3, KCNIP1, LAMA1, LOC339240, MEGF11, PLCXD3, RBP4, SFN, SHANK2, WIF1, FGF18, UBD, ANGPTL7 and SLC2A4.

Detection of the hedgehog biomarkers can be carried out by standard histological and/or immuno-detection methods. In one embodiment, the hedgehog-associated marker can be detected by any means of polypeptide detection, or detection of the expression level of the polypeptides. For example, the polypeptide can be detected using any of antibody detection methods (e.g., immunofluorescent (IF) methods, immunofluorescence cell sorting (FACS)), antigen retrieval and/or microarray detection methods can be used. A reagent that specifically binds to a hedgehog-associated marker polypeptide, e.g., an antibody, and antibody derivative, and an antibody fragment, can be used. Other detection techniques that can be used include, e.g., capture assays (e.g., ELISA), mass spectrometry (e.g., LCMS/MS), and/or polymerase chain reaction (e.g., RT-PCR). The hedgehog-associated biomarkers can also be detected by systemic administration of a labeled form of an antibody to the hedgehog-associated biomarkers followed by imaging. In another embodiment, the nucleic acid sample from the subject is evaluated by a nucleic acid detection technique as described herein.

In one embodiment, the detection of the level of the hedgehog-associated biomarker includes contacting the sample with a reagent, e.g., an antibody that binds to the hedgehog biomarker, such as a component of a cilium (e.g., an anti-acetylated tubulin antibody) and detecting the level of the reagent, e.g., an antibody, bound to the biomarker. The antibody can be labeled with a detectable label (e.g., a fluorescent or a radioactive label) or can be detected using a fluorescently labeled secondary antibody. In one embodiment, the amount, structure and/or activity of the one or more hedgehog-associated marker polypeptides can be compared to a reference value, e.g., a control sample. In one embodiment, detection of the biomarker includes use of scanning- or transmission-electron microscopy or electron tomography using standard methods.

In certain embodiments where the one or more alterations in the hedgehog-associated marker(s) involve nucleic acid alterations, the alterations are detected by any method of detection available in the art, including but not limited to, nucleic acid hybridization assay, amplification-based assays (e.g., polymerase chain reaction such as a reverse transcription polymerase chain reaction (RT-PCR) assay), sequencing, screening analysis (including metaphase cytogenetic analysis by standard karyotype methods, FISH, spectral karyotyping or MFISH, and comparative genomic hybridization), and/or in situ hybridization. In one embodiment, the amount, structure and/or activity of the one or more hedgehog marker nucleic acid (e.g., DNA or RNA) can be compared to a reference value or sample, e.g., a control sample.

In one embodiment, the method includes: contacting a sample, e.g., a genomic DNA sample (e.g., a chromosomal sample or a fractionated, enriched or otherwise pre-treated sample) or a gene product (mRNA), obtained from the subject with a probe (e.g., an exon-specific probe, a probes specific for the desired sequence) under conditions suitable for hybridization, and determining the presence or absence of one or more of the abnormalities in the gene or gene product (e.g., genomic DNA in chromosomal regions associated with cytogenetic abnormalities. The method can, optionally, include enriching a sample for the gene or gene product.

Nuclear Gli1 can be detected using art known techniques, including immunohistochemistry and immunofluorescence.

In one embodiment, the detection of the hedgehog-associated biomarker includes detection of one or more hedgehog ligands, or detection of the expression level of the one or more hedgehog ligands. For example, the amount of one or more of SHh, IHh or DHh in a sample (e.g., in a subject's plasma or sera) can be quantified. Hedgehog ligand expression can be measured by detection of a soluble form of the ligand in peripheral blood and/or urine (e.g., by an ELISA assay or radioimmunoassay), in circulating tumor cells (e.g., by a fluorescence-activated cell sorting (FACS) assay, or an immunohistochemistry assay), or in tumor or bone marrow biopsies (e.g., by an immunohistochemistry assay, a RT-PCR assay, or by in situ hybridization). Detection of hedgehog ligand in a given patient tumor could also be assessed in vivo, by systemic administration of a labeled form of an antibody to a hedgehog ligand followed by imaging.

In one embodiment, the hedgehog ligand levels can be quantified by detecting a signature peptide common to at least two hedgehog ligands, e.g., a peptide having the amino acid sequence: AVEAGF (SEQ ID NO: 4), or an amino acid sequence substantially identical thereto (e.g., having one, two or three substitutions, e.g., conservative substitutions). The method includes (optionally) treating the sample (e.g., plasma or serum sample) with an agent that breaks at least one peptidic bond, e.g., a protease, thereby releasing one or more peptide products; detecting the presence or amount of the signature peptide (e.g., a peptide having the amino acid sequence: AVEAGF (SEQ ID NO: 4), or an amino acid sequence substantially identical thereto), thereby identifying one or more peptides having the signature peptide sequence. The detection step can include the step of analysis of the digested peptide product by one or more of mass spectrometry (e.g., LC-MS/MS), mapping, sequencing, and/or antibody based detection methods (e.g., ELISA, RIA, Western blot). For example, the sample can be digested with a protease and the digested peptide can be analyzed by mass spectrometry (e.g., LC-MS/MS), mapped, and/or sequenced.

In yet other embodiments, the detection of the hedgehog-associated marker involves detection of an alteration in tumor architecture, including but not limited to, measurement of tumor size and/or tumor perfusion. In one embodiment, a change in tumor perfusion is evaluated before and after dosing with a hedgehog inhibitor and/or a chemotherapeutic agent (e.g., pre- and after treatment with gemcitabine and/or a hedgehog inhibitor (e.g., IPI-926)). For example, the tumor uptake of a labeling agent (e.g., Gd-DTPA) can be detected before and after treatment with the hedgehog inhibitor and/or the therapeutic agent. In other embodiments, the tumor perfusion can be detected using Magnetic Resonance Imaging (MRI), positron emission tomography (PET), or other imaging techniques.

In certain embodiments, the detection of the hedgehog-associated biomarker (e.g., detection of the presence of a mutation in a gene or gene product, increase in the level of expression, or increase in tumor size) in the methods or assays of the invention is indicative of a pre-determined clinical outcome, prognosis, and/ diagnosis. In other embodiments, detection of the presence of a mutation in a gene or gene product, or a change (e.g., an increase or decrease) in the level of a marker of a hedgehog pathway (e.g., WIF, Gli1 (e.g., nuclear Gli1)); presence or absence of primary cilia or a phosphorylated hedgehog receptor (e.g., phosphorylated Smo) in the cilia; a genomic marker or an EMT marker (e.g., an increase in any of (i) to (iii) above (e.g., an increased level in a hedgehog ligand; the presence of an SNP of a hedgehog ligand, detection; or a change (e.g., an increase or decrease) or detection of a mutation in KRAS, TGFβ-SMADs, p53, cyclin D1, or Gli1) is indicative of a positive clinical outcome upon administration of a hedgehog inhibitor (e.g., is indicative of the likelihood that a subject with cancer will respond to a hedgehog inhibitor).

In other embodiments, a change (e.g., an increase or decrease) in one of more of Gemcitabine markers (e.g., a marker as described in (iv) above) is indicative of a positive outcome (e.g., administration of a hedgehog inhibitor can lead to an increase in Gemcitabine uptake by the tumor cell), thus increasing the beneficial therapeutic effects of Gemcitabine, alone or in combination with a hedgehog inhibitor. Likewise, detection of one or more of: increased tumor perfusion, interstitial fluid pressure, alteration microvascular density (MVD), and increased permeability of tumor-associated stroma can be indicative of a positive clinical outcome in response to administration of the hedgehog inhibitor.

In one embodiment, the sample is collected or obtained from the subject. For example, the sample has been previously collected by a medical practitioner. Alternatively, the method, or assay, further includes obtaining or collecting a sample from the subject. The sample can be chosen from one or more of: tissue, whole blood, serum, plasma, buccal scrape, saliva, cerebrospinal fluid, urine, stool, circulating tumor cells, circulating nucleic acids, or bone marrow. In some embodiments, the sample is a serum, plasma, or tissue sample from the subject, e.g., a human cancer patient (e.g., a human patient with pancreatic cancer). In some embodiments, the sample is a formalin-fixed paraffin-embedded (FFPE) tissue sample, or a frozen sample. The sample can be collected from a biopsy or surgery. The FFPE or frozen samples can be analyzed for, e.g., immunohistochemistry (IHC) or histopathology (such as hematoxylin and eosin (HE) stain, hedgehog ligand detection, Gli-1 and/or -2 detection, desmoplasia, MVD, or SMA immunohistochemistry), or DNA extraction. In other embodiments, the sample is a plasma or serum sample (e.g., for use in an ELISA or proteomics assay). In yet other embodiments, the sample is a whole blood sample, e.g., for germline DNA extraction to evaluate SNPs.

In certain embodiments, the methods, or assays, of the invention include the step of analyzing a nucleic acid or a protein from the subject, e.g., analyzing the genotype of the subject. In one embodiment, detection of one or more cilium markers, or an elevated level of a hedgehog-associated biomarker is detected (e.g., an elevated level of a hedgehog ligand protein, or a nucleic acid encoding a hedgehog ligand; and/or an elevated or decreased upstream or downstream component(s) of the hedgehog signaling (e.g., a hedgehog receptor, or a hedgehog signaling mediator (e.g., Gli1, Gli2, and Gli3) as described herein)). The marker can be detected in blood, plasma, serum, urine, circulating tumor cells, a tumor biopsy or a bone marrow biopsy.

In certain embodiments, the methods, or assays, of the invention include the step of detecting the one or more hedgehog-associated biomarkers in the subject, prior to, or after, administering a hedgehog inhibitor and/or a chemotherapeutic agent to the subject, e.g., the patient.

The hedgehog-associated biomarker can be measured at least at two time-points. For example, the hedgehog-associated biomarker can be measured pre- and post-chemotherapy, pre-chemotherapy and at one or more time-points while chemotherapy is ongoing, or at two or more different time-points while chemotherapy is ongoing. If the hedgehog biomarker is found to be present or up-regulated, a hedgehog inhibitor can be administered. Thus, measurement of the hedgehog-associated biomarker in the patient can determine whether the patient receives a hedgehog pathway inhibitor in combination with or following other chemotherapy.

In certain embodiments, the step of detecting the hedgehog-associated biomarker(s) can include the steps of measuring the biomarker(s) in the patient prior to administration of other cancer therapy, measuring the biomarker(s) in the patient after administration of other cancer therapy, and determining if the amount of hedgehog biomarker(s) after administration of the other chemotherapy is greater than the amount of the hedgehog-associated biomarker(s) before administration of the other chemotherapy. The other cancer therapy can be, for example, a chemotherapeutic or radiation therapy.

In yet another embodiment, the hedgehog-associated markers are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to metastatic disease. In another embodiment, the significant event is the progression from primary diagnosis to relapse. In another embodiment, the significant event is the progression from metastatic disease to death. In another embodiment, the significant event is the progression from metastatic disease to relapse. In another embodiment, the significant event is the progression from relapse to death. In certain embodiments, the time course is measured with respect to one or more overall survival rate, time to progression and/or using the Response Evaluation Criteria in Solid Tumors (RECIST) or other response criteria.

In certain embodiments, a predetermined measure is created after evaluating the sample by dividing subject's samples into at least two patient subgroups. In certain embodiments, the number of subgroups is two so that the patient sample is divided into a subgroup of patients having the one or more abnormalities, e.g., an alteration in one or more of the hedgehog-associated biomarkers described herein, and a subgroup not having the abnormalities. In certain embodiments, the hedgehog biomarker status in the subject is compared to either the subgroup having or not having an alteration in the hedgehog biomarker(s); if the patient has an alteration the hedgehog marker(s), then the patient is likely to respond to an hedgehog inhibitor (e.g., IPI-926) and/or the patient has an increased likelihood, or is likely, to have a long time course. In certain embodiments, the number of subgroups is greater than two, including, without limitation, three subgroups, four subgroups, five subgroups and six subgroups, depending on stratification of predicted hedgehog inhibitor efficacy as correlated with particular abnormalities. In certain embodiments, likeliness to respond is measured with respect to overall survival rate, time to progression and/or using the RECIST criteria. In certain embodiments, the hedgehog inhibitor is IPI-926.

In other embodiments, the method, or assay, further includes the step of identifying one or more therapeutic agents that elevate the hedgehog-associated marker (e.g., elevate the level, expression or subcellular localization of one or more hedgehog-associated markers as described herein). The methods can include the step of administering a therapeutically effective amount of the one or more therapeutic agents that elevate the hedgehog-associated marker and a therapeutically effective amount of a hedgehog inhibitor. The step of identifying the therapeutic agent that elevate the hedgehog-associated biomarker can include the steps of exposing cells from the tumor to one or more therapeutic agents in vitro and measuring the presence or expression of the hedgehog-associated biomarker in the cells.

In certain embodiments, the subject identified or treated is a mammal, e.g., a primate, typically, a human (e.g., a patient having, or at risk of, a cancer or tumor described herein). The subject can be one at risk of having the disorder, e.g., a subject having a relative afflicted with the cancer, or a subject having a genetic trait associated with risk for the cancer. In one embodiment, the subject can be symptomatic or asymptomatic. In certain embodiments, the subject is a patient having one or more alterations in a hedgehog biomarker, e.g., an alteration in primary cilia or a phosphorylated hedgehog receptor (e.g., phosphorylated Smo) in the cilia, a marker of a hedgehog pathway, an alteration in a genomic marker, an alteration in a marker of Epithelial to Mesenchymal Transition (EMT), an alteration in a Gemcitabine marker, and/or an alteration in tumor architecture.

Additional embodiments or features of the present invention are as follows.

In another aspect, the invention provides a method of, or assay for, evaluating a sample, e.g., a sample obtained from a cancer patient, to detect the presence or amount of a signature peptide common to at least two hedgehog polypeptides, e.g., a peptide having the amino acid sequence: AVEAGF (SEQ ID NO: 4), or an amino acid sequence substantially identical thereto (e.g., having one, two or three substitutions, e.g., conservative substitutions). The method includes (optionally) treating the sample (e.g., plasma or serum sample) with an agent that breaks at least one peptidic bond, e.g., a protease, thereby releasing one or more peptide products; detecting the presence or amount of the signature peptide (e.g., a peptide having the amino acid sequence: AVEAGF (SEQ ID NO: 4), or an amino acid sequence substantially identical thereto), thereby identifying one or more peptides having the signature peptide sequence. The detection step can include the step of analysis of the digested peptide product by one or more of mass spectrometry (e.g., LC-MS/MS), mapping, sequencing, and/or antibody based detection methods (e.g., ELISA, RIA, Western blot).

In a related aspect, the invention features a peptide consisting of the amino acid sequence: AVEAGF (SEQ ID NO: 4) or an amino acid sequence substantially identical thereto (e.g., having one, two or three substitutions, e.g., conservative substitutions); as well as capture reagents, e.g., antibodies, that specifically bind to the AVEAGF (SEQ ID NO: 4), or an amino acid sequence substantially identical thereto.

Alternatively, or in combination with the methods described herein, the invention features a method of treating a cancer or tumor harboring one or more hedgehog-associated biomarkers described herein, with a hedgehog inhibitor, alone or in combination with other therapeutic agents and/or radiation. The method includes administering to the subject one or more hedgehog inhibitor(s) as described herein, in an amount sufficient to reduce or inhibit the tumor cell growth, and/or treat or prevent the cancer(s), in the subject.

In another aspect, alternatively, or in combination with the methods described herein, the invention features a method of enhancing delivery of a therapeutic agent(s), to a target site, e.g., a tumor or a cancer cell. The method includes contacting a tumor or a cancer cell with, or administering to a subject, a hedgehog inhibitor (e.g., one or more hedgehog inhibitors as described herein), alone or in combination with the therapeutic agent, in an amount sufficient to increase the delivery of the therapeutic agent(s) to the target site. In one embodiment, the hedgehog inhibitor causes a decrease in interstitial fluid pressure. The hedgehog inhibitor can be administered prior to, or concurrently with, the therapeutic agent.

In yet another aspect, alternatively, or in combination with the methods described herein, the invention features a method of reducing interstitial pressure in a tumor or a cancer cell. The method includes contacting a tumor or cancer cell, or administering to a subject, a hedgehog inhibitor (e.g., one or more hedgehog inhibitors as described herein), in an amount sufficient to decrease the interstitial fluid pressure in the tumor or cancer cell. The hedgehog inhibitor can be administered prior to, or concurrently with, the therapeutic agent(s).

In another aspect, alternatively, or in combination with the methods described herein, the invention features a method of targeting delivery of a hedgehog inhibitor and/or a therapeutic agent(s) to a hedgehog-responsive cell. The method includes contacting a hedgehog-responsive cell, e.g., a pericyte, with a hedgehog inhibitor (e.g., one or more hedgehog inhibitors as described herein) and/or a therapeutic agent, wherein the hedgehog inhibitor and/or the therapeutic agent are selectively targeted to the hedgehog-responsive cell. In one embodiment, the hedgehog inhibitor and/or the therapeutic agent are associated with (e.g., coupled to) an agent that binds to the hedgehog-responsive cell (e.g., an antibody or a fragment thereof that binds to a surface molecule in the hedgehog-responsive cell). In one embodiment, the agent is an antibody molecule or fragment thereof that binds to a PDGF receptor, e.g., PDGFR-beta, present on the surface of the hedgehog-responsive cell, e.g., a pericyte.

Treatment as referred to herein can include, but is not limited to, inhibiting tumor growth, reducing tumor mass, reducing size or number of metastatic lesions, inhibiting the development of new metastatic lesions, prolonged survival, prolonged progression-free survival, prolonged time to progression, and/or enhanced quality of life.

In certain embodiments, the cancer or tumor evaluated or treated by the methods, or assays, of the invention includes, but is not limited to, a solid tumor, a soft tissue tumor, and a metastatic lesion (e.g., a cancer as described herein). In some embodiments, the cancer identified or treated harbors a hedgehog-associated biomarker (e.g., a cilium and/or other hedgehog-associated biomarker as described herein).

Exemplary cancers that can be treated include, but are not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphoblastic leukemia (ALL), acute myeloid leukemia (AML), chronic myelogenous leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), multiple myeloma (MM), myelodysplastic syndrome (MDS), myeloproliferative disorder (MPD) (e.g., polycythemia Vera (PV), essential thrombocythemia (ET), agnogenic myeloid metaplasia (AMM) a.k.a. primary myelofibrosis (PMF), chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (KA), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, rhabdomyosarcoma, myxosarcoma).

In certain embodiments, the cancer or tumor that can be treated is selected from bladder cancer, breast cancer, medulloblastoma, colorectal cancer, head and neck cancer, lung cancer (e.g., small cell lung cancer (SCLC), non-small cell lung cancer (NSCLC)), leukemia (e.g., acute lymphoblastic leukemia (ALL), acute myeloid leukemia (AML), chronic myelogeneous leukemia (CML), chronic lymphocytic leukemia (CLL)), lymphoma (e.g., Hodgkin lymphoma (HL), non-Hodgkin lymphoma (NHL)), multiple myeloma (MM), chronic myeloproliferative disorder (primary myelofibrosis, polycythemia vera, essential thrombocytemia), osteosarcoma, ovarian cancer, pancreatic cancer, prostate cancer, basal cell carcinoma (BCC)) or chondrosarcoma.

In other embodiments, the methods, assays, and/or kits described herein further include providing and/or transmitting information, e.g., a report, containing data of the evaluation or treatment determined by the methods, assays, and/or kits as described herein to a report-receiving party or entity (e.g., a patient, a health care provider, a diagnostic provider, and/or a regulatory agency, e.g., the FDA), or otherwise submitting information about the methods, assays and kits disclosed herein to another party. The method can relate to compliance with a regulatory requirement, e.g., a pre- or post approval requirement of a regulatory agency, e.g., the FDA. In one embodiment, the report-receiving party or entity can determine if a predetermined requirement or reference value is met by the data, and, optionally, a response from the report-receiving entity or party is received, e.g., by a physician, patient, diagnostic provider.

In another aspect, the invention features a method of treating a patient having cancer or at risk for developing cancer. The method includes: (optionally) (a) providing or collecting a sample from a subject, e.g., a sample and a subject as described herein; (b) evaluating the sample to detect the presence of a marker as described herein (e.g., one or more hedgehog-associated biomarkers as described herein); and (c) administering to said subject a therapeutically effective amount of a hedgehog inhibitor (e.g., one or more hedgehog inhibitors), alone or in combination with other agents as described herein.

In another aspect, the invention features a method of reducing or inhibiting growth of one or more tumors in a subject. The invention also features a method of treating a subject having, or at risk of having, a cancer or tumor having a hedgehog-associated biomarker (e.g., one or more hedgehog-associated biomarker as described herein). The method includes evaluating a sample to detect a hedgehog-associated biomarker (e.g., one or more of the hedgehog-associated biomarker described herein); and administering to the subject a hedgehog inhibitor, e.g., one or more of the hedgehog inhibitors as described herein, alone or in combination with other therapeutic agents as described herein, in an amount sufficient to reduce or inhibit the tumor cell growth, and/or treat or prevent the cancer(s), in the subject. The subject can have a hedgehog marker chosen from one or more of, e.g., primary cilia or a component thereof, or a phosphorylated hedgehog receptor (e.g., phosphorylated Smo) in the cilia; a hedgehog pathway (e.g., nuclear Gli1 expression); an alteration in a genomic marker; an alteration in a marker of Epithelial to Mesenchymal Transition (EMT), an alteration in a Gemcitabine marker, and/or an alteration in tumor architecture. In certain embodiments, the subject has a cancer as described herein. In certain embodiments, the cancer treated includes, but is not limited to, a solid tumor, a soft tissue tumor, and a metastatic lesion (e.g., a cancer as described herein). In certain embodiments, the subject has a cancer selected from bladder cancer, breast cancer, medulloblastoma, colorectal cancer, head and neck cancer, lung cancer (e.g., small cell lung cancer (SCLC), non-small cell lung cancer (NSCLC)), leukemia (e.g., acute lymphoblastic leukemia (ALL), acute myeloid leukemia (AML), chronic myelogeneous leukemia (CML), chronic lymphocytic leukemia (CLL)), lymphoma (e.g., Hodgkin lymphoma (HL), non-Hodgkin lymphoma (NHL)), multiple myeloma (MM), osteosarcoma, ovarian cancer, pancreatic cancer, prostate cancer, basal cell carcinoma (BCC)) and chondrosarcoma. In certain embodiments, the subject has been previously evaluated for the presence of one or more alterations in an oncogenic marker (e.g., KRAS, EGFR, HER2, ALK) and/or a cilium or other hedgehog-associated marker as described herein.

The methods of the invention can further include the step of monitoring the subject, e.g., for a change (e.g., an increase or decrease) in one or more of: tumor size; hedgehog levels, signaling or subcellular localization; stromal activation; levels of one or more cancer markers; the rate of appearance of new lesions, e.g., in a bone scan; the appearance of new disease-related symptoms; the size of soft tissue mass, e.g., a decreased or stabilization; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same hedgehog inhibitor, alone or in combination with, the same therapeutic agent, or for additional treatment with other additional therapeutic agents and/or radiation. Generally, a decrease in one or more of the parameters described above is indicative of the improved condition of the subject, although with serum hemoglobin levels, an increase can be associated with the improved condition of the subject.

In another aspect, the invention features a kit for evaluating a sample, e.g., a sample from a cancer patient, to detect a hedgehog-associated biomarkers as described herein (e.g., nuclear Gli1; the presence of one or more of cilium marker (e.g., a cilium or a component thereof); a marker of a hedgehog pathway (e.g., nuclear Gli1); a genomic marker; a marker of Epithelial to Mesenchymal Transition (EMT); a Gemcitabine marker; and/or an alteration in tumor architecture). In one embodiment, a kit for determining the chemosensitivity of a subject, e.g., a cancer patient, to treatment with a hedgehog inhibitor is provided. The kit includes a means for detection of (e.g., a reagent that specifically detects) the hedgehog biomarker as described herein. In certain embodiments, the kit includes a hedgehog inhibitor, alone or in combination with a therapeutic agent. In one another embodiment, the kit comprises an antibody, and antibody derivative, and an antibody fragment to a hedgehog biomarker polypeptide (e.g., a cilium or a component thereof, a hedgehog signaling component (e.g., Gli1) and/or a hedgehog ligand). In one embodiment, the kit includes an antibody-based detection technique, such as immunofluorescence cell sorting (FACS), immunohistochemistry, antigen retrieval and/or microarray detection reagents.

In one embodiment, at least one of the reagents in the kit is an antibody that binds to a component of a cilium (e.g., an anti-tubulin antibody) (optionally) with a detectable label (e.g., a fluorescent or a radioactive label). In certain embodiments, the kit is an ELISA or an IHC assay for detection of a cilium marker, a hedgehog ligand, Gli-1, Gli-2, SPARC, and/or EMT markers. For example, the kit can detect the amount of one or more of SHh, IHh or DHh in a sample (e.g., in a subject's plasma or sera). In one embodiment, the hedgehog ligand levels can be detected by evaluating the presence or amount of a signature peptide in common with at least two hedgehog ligands, e.g., a peptide having the amino acid sequence: AVEAGF (SEQ ID NO: 4), or an amino acid sequence substantially identical thereto (e.g., having one, two or three substitutions, e.g., conservative substitutions). For example, the kit can contain reagents for digesting a sample with a protease and analyzing the digested peptide by one or more of: mass spectrometry (e.g., LC-MS/MS), mapping, and/or sequencing techniques.

In another embodiment, the reagent of the kit comprises one or more polynucleotide probes (e.g., a polynucleotide sequence which is complementary to a nucleotide sequence encoding a cilium marker as described herein, or a complementary sequence thereto). In another embodiment, the probes comprise polynucleotides from 10, 20, 30, 40, or 50 to 10⁷ nucleotides in length. In yet another embodiment, the probes are selected from the group consisting of oligonucleotides, cDNA molecules, RNA molecules, and synthetic gene probes comprising nucleobases. In other embodiment, the probes include exonic sequence, or sequences complementary thereto.

In embodiments, the sample is evaluated in relation to a reference value, e.g., a control sample. The kit can optionally include instructions for use in detecting the alterations, and/or evaluating the results.

In one embodiment, the hedgehog inhibitor used in the methods or compositions of the invention is a compound of formula:

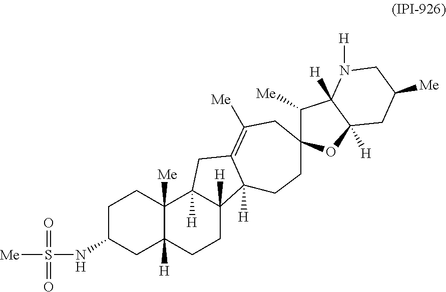

or a pharmaceutically acceptable salt thereof. A compound of formula above, or a pharmaceutically acceptable salt thereof, is also referred to herein as IPI-926. An example of a pharmaceutically acceptable salt of the compound of formula I is the hydrochloride salt.

In some embodiments, the hedgehog inhibitor is administered as a pharmaceutical composition comprising the hedgehog inhibitor, or a pharmaceutically acceptable salt thereof, and a pharmaceutically acceptable excipient.

In certain embodiments, one or more hedgehog inhibitors are administered, or are present in the composition, e.g., the pharmaceutical composition.

The hedgehog inhibitors described herein can be administered to the subject systemically (e.g., orally, parenterally, subcutaneously, intravenously, rectally, intramuscularly, intraperitoneally, intrathecally, intranasally, transdermally, or by inhalation or intracavitary installation). Typically, the hedgehog inhibitors are administered orally.

In one embodiment, the hedgehog inhibitor is IPI-926. IPI-926 can be administered orally in a daily schedule at a dose of about 20 mg to 200 mg, typically about 50 to 150 mg, 75 to 140 mg, and more typically 120 to 130 mg, alone or in combination with a second agent as described herein.

The methods and compositions of the invention can, optionally, be used in combination with one or more other cancer therapies (e.g., one or more therapeutic agents surgery and/or radiation). Thus, the methods of the invention can include the steps of administering to the subject in need of treatment, or at risk of having the cancer, a hedgehog inhibitor as described herein, in combination with one or more cancer therapies as described herein, in an amount effective to reduce or treat the cancer, e.g., a cancer as described herein.

Any combination of the hedgehog inhibitor and other cancer therapy can be used. For example, the hedgehog inhibitor and other cancer therapy can be administered during periods of active disorder, or during a period of remission or less active disease. The hedgehog inhibitor and other cancer therapy can be administered before treatment, concurrently with treatment, post-treatment, or during remission of the disorder. In one embodiment, the cancer therapy is administered simultaneously or sequentially with the hedgehog inhibitor. In certain embodiments, the cancer therapy is radiation. In certain embodiments, the cancer therapy is surgery. In certain embodiments, the cancer therapy is a therapeutic agent (e.g., a biotherapeutic agent or chemotherapeutic agent).

In other embodiments, the hedgehog inhibitor and the therapeutic agent are administered as separate compositions, e.g., pharmaceutical compositions. In other embodiments, the hedgehog inhibitor and the therapeutic agent are administered separately, but via the same route (e.g., both orally or both intravenously). In still other instances, the hedgehog inhibitor and the therapeutic agent are administered in the same composition, e.g., pharmaceutical composition.

In one embodiment, hedgehog inhibitor is administered in combination with an anti-cancer agent (e.g., a cytotoxic or a cytostatic agent). In one embodiment, the anti-cancer agent is chosen from a tyrosine kinase inhibitor, a taxane, gemcitabine, cisplatin, epirubicin, 5-fluorouracil, a VEGF inhibitor, leucovorin, oxaplatin, Ara-c, or a combination thereof. In other embodiments, the anti-cancer agent is chosen from one or more of an insulin-like growth factor receptor (IGF-1R) inhibitor, a PI3K inhibitor, an HSP90 inhibitor, folfirinox, a BRAF inhibitor, a MEK inhibitor, or a JAK2 inhibitor. Exemplary tyrosine kinase inhibitors include, but are not limited to, sunitinib, erlotinib, gefitinib, sorafenib, icotinib, lapatinib, neratinib, vandetanib, BIBW 2992 or XL-647. Other tyrosine kinase inhibitor can be chosen from a monoclonal antibody against EGFR, e.g., cetuximab, panitumumab, zalutumumab, nimotuzumab necitumumab or matuzumab. Additional exemplary combination therapies are described herein.

In one embodiment, the hedgehog inhibitor (e.g., IPI-926) is administered in combination with a PI3K inhibitor. In one embodiment, the PI3K inhibitor is an inhibitor of delta and gamma isoforms of PI3K. Exemplary PI3K inhibitors that can be used in combination are described in, e.g., WO 09/088,990; WO 09/088,086; WO 2011/008302; WO 2010/036380; WO 2010/006086, WO 09/114,870, WO 05/113556; US 2009/0312310, US 2011/0046165. Additional PI3K inhibitors that can be used in combination with the hedgehog inhibitors, include but are not limited to, GSK 2126458, GDC-0980, GDC-0941, Sanofi XL147, XL756, XL147, PF-46915032, Novartis BEZ 235, BKM 120, CAL-101, CAL 263, SF1126 and PX-886. In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof. The hedgehog inhibitor and the PI3K inhibitor can be administered simultaneously or sequentially as described herein. In certain embodiments, the inhibitors are administered in the same composition, or in different compositions, as described hereinbelow.

In certain embodiments, the therapeutic agent is gemcitabine.

In another embodiment, the therapeutic agent is a paclitaxel agent. In certain embodiments, the paclitaxel agent is a paclitaxel equivalent. In certain embodiments, the paclitaxel equivalent is ABRAXANE®. In yet other embodiments, the hedgehog inhibitor, alone or combination with the therapeutic agent is administered in a therapeutically effective amount, e.g., at a predetermined dosage schedule.

In certain embodiments, wherein the hedgehog inhibitor is used in combination with a therapeutic agent, the method includes administering the hedgehog inhibitor and/or the therapeutic agent at sub-cytotoxic levels.

In another aspect, the invention features a composition, e.g., a pharmaceutical composition that includes one or more hedgehog inhibitors, e.g., a hedgehog inhibitor as described herein, and one or more therapeutic agents.

All publications, patent applications, patents, and other references mentioned herein are incorporated by reference in their entirety.

Other features, objects, and advantages of the invention will be apparent from the description and drawings, and from the claims.

### DESCRIPTION OF THE FIGURES

The application file contains at least one drawing executed in color. Copies of this patent application publication with color drawing(s) will be provided by the Office upon request and payment of the necessary fee.

FIG. 1A is a bar graph depicting the level of Gli-1 mRNA expression normalized to human GAPDH in HT1080 fibrosarcoma cells and SW872 liposarcoma cells, each one in two control samples (C), two samples treated with Sonic Hedgehog (SHh), and two samples treated with SHh and 500 nM of the hedgehog inhibitor, IPI-926.

FIG. 1B is a bar graph depicting the changes in human and murine Gli-1 mRNA expression in response to treatment with IPI-926.

FIG. 2 is a bar graph depicting the level of Gli-1 mRNA expression normalized to human GAPDH in MG-63 osteosarcoma cells in two control samples (C), two samples treated with Sonic Hedgehog (SHh), and two samples treated with SHh and 500 nM of the hedgehog inhibitor, IPI-926.

FIG. 3 is a bar graph depicting the level of Gli-1 mRNA expression normalized to human GAPDH in GCT and SK-LMS-1 sarcomas cells, each one in two control samples (C), two samples treated with Sonic Hedgehog (SHh), and two samples treated with SHh and 500 nM of the hedgehog inhibitor, IPI-926.

FIG. 4 is a bar graph depicting the level of Gli-1 mRNA expression normalized to human GAPDH in SW982 synovial sarcoma cells in two control samples (C), two samples treated with Sonic Hedgehog (SHh), and two samples treated with SHh and 500 nM of the hedgehog inhibitor, IPI-926.

FIGS. 5A-5B are tables summarizing the cell line information, and experimental data on cilia IF, Hh response and control vimentin IF for multiple cell lines tested.

FIG. 6 depicts a panel of immunohistochemical stains of Basal Cell Carcinoma (BCC) tissue samples using immunofluorescence (IF) with antibodies specific for acetylated-tubulin FIGS. 7A-7E are a series of panels and a table depicting expression of Sonic hedgehog (SHh) in primary tumors and xenograft models. FIGS. 7A-7D show photographs of pancreatic ductal adenocarcinoma, colon adenocarcinoma, ovarian cystadenocarcinoma and prostate adenocarcinoma, respectively. FIG. 7E is a table summarizing the level of expression of SHh in the tissues shown in FIGS. 7A-7D.

FIGS. 8A-8B show a timecourse of increased human SHh expression in bladder cells treated with gemcitabine and doxorubicin.

FIGS. 8C-8D show Western blots corresponding to the timecourse shown in FIGS. 8A-8B.

FIGS. 9A and 9B show upregulation of expression of human IHh ligand in after chemotherapy of LX-22 tumors, whereas expression of murine Gli-1 in the tumor stroma was decreased after chemotherapy.

FIGS. 10A-10D depict images from ultrasound measurements of blood perfusion in xenografts.

FIG. 11 depicts an amino acid sequence comparison of SHh, IHh and DHh (SEQ ID NOs:1-3, respectively).

FIGS. 12A-12B depict the responsiveness of stroma in L3.6pl cells to IPI-926. FIG. 12A is a bar graph depicting the inhibition of stromal Gli1 expression in IPI-926-treated tumors (treated with 40 mg/kg or IPI-926) compared to vehicle. FIG. 12B is a photograph depicting the level of SHh ligand by immunohistochemistry (IHC).

FIG. 13 is a linear graph depicting the increased tumor growth inhibition (detected as tumor volume after days of treatment post implant) in the animals treated with the combination of IPI-926 and albumin-bound paclitaxel (Abraxane®) in L3.6pl pancreatic xenograft model.

FIGS. 14A-14E depict the effects of IPI-926 in tumor perfusion in a subcutaneous L3.6pl tumor model. Figures A and D depict control (vehicle-treated) and Figures B and D show results from IPI-926-treated animals. The results are tabulated in FIG. 14E. The time to reach peak contrast was measured and showed a decrease in IPI-926-treated animals compared to vehicle (FIGS. 14C and 14D, and tabulated in FIG. 14E.

FIG. 15 is a bar graph depicting the paclitaxel levels in tumor tissue.

FIG. 16 shows two bar graphs depicting the level of IPI-926-responsiveness in Lin+ and Lin− cells, and CD31± and CD31− cells, as detected by evaluating mGli-1 expression.

FIG. 17 is a bar graph depicting the effects of IPI-926, alone or in combination with Bevacizumab measured by changes in tumor size as a function of days of treatment post-implant.

FIGS. 18A-18B are bar graphs depicting a comparison of pericyte-specific (RGS5) and endothelial-specific (PECAM-1) marker mRNA expression from BxPC3 tumors in mice treated with IPI-926, alone of in combination with Bevacizumab.

FIG. 19 is a micrograph depicting tumor cells stained for cilia. A human bone and cartilage tumor tissue array (Biomax™) was stained for cilia by immunofluorescence. The majority of the cilia (white arrow) were found on the chondrosarcoma tumor cells.

FIG. 20 is a table showing that detection of primary cilia is associated with responsiveness to hedgehog inhibition in a panel of sarcoma cell lines.

FIGS. 21A-21C show that IPI-926 inhibits tumor growth in multiple patient derived tumor xenograft models. Mice received daily treatment of IPI-926, M-F, for 6-10 weeks (n=10-15 per group). The range of tumor growth inhibition is 33-52%, with a mean of 43%.

FIGS. 22A-22D are micrographs depicting changes in morphology observed with IPI-926 treatment, including loss of tumor cells and calcification of the chondroid matrix.

FIGS. 23A-23B show images of Basal Cell Carcinoma (BCC) lesion in a patient before and after IPI-926 treatment. A reduction in the size and appearance of the lesion is detected in the patient prior to treatment (FIG. 23A) compared to the size of the same lesion six months after treatment with IPI-926 (FIG. 23B).

FIGS. 23C-23D show images of nuclear Gli1 staining in biopsy samples of a BCC lesion pre- and post-IPI-926 treatment (22 days post-treatment).

FIGS. 24A-24B show images of hematoxylin and eosin staining in biopsy samples of a BCC lesion pre- and post-IPI-926 treatment, respectively.

FIGS. 25A-25B show images of Gli-1 immunohistochemical staining of BCC lesions pre- and post-IPI-926 treatment, respectively. The H score of the pre-treated sample (A) was 100, whereas the post-treated sample (B) had an H score of 57.

FIG. 26 shows an image of stromal nuclear Gli1 staining from a pancreatic tumor sample. Representative stromal nuclear Gli1 staining is shown as a darker stain in the region labeled as Gli-1 positive stromal cells. Tumor cells are indicated by the arrows. Image is shown at a 400× magnification.

FIG. 27 is a bar graph showing increased Gli1 mRNA expression in response to SHh addition to pericytic C3H10T1/2 cells in culture. The increased Gli1 mRNA expression was inhibited by co-addition of SHh and IPI-926.

FIG. 28 is a bar graph depicting the regulation of the indicated markers (NG2. RGS5. CD13 and N-cadherin) in pericytic C3H10T1/2 cells in culture treated under the conditions shown, namely, control, SHh, SHh+IPI-926, and IPI-926. Expression of RGS5 was downregulated by SHh in these cells, and this effect was reversed by addition of IPI-926.

### DETAILED DESCRIPTION

Malignant activation of the Hedgehog (Hh) pathway is associated with multiple tumor types and can promote the growth of certain cancers via at least three mechanisms of action: Hh ligand-dependent signaling between tumor cells, Hh ligand-dependent signaling between tumor cells and their microenvironment, and ligand-independent signaling caused by mutations in the Hh receptors Patched or Smoothened. Hedgehog inhibitors can target tumors directly, e.g., by inhibiting oncogenic signaling and/or tumor cell apoptosis. Examples of such tumors can be ligand-independent (e.g., Patched mutant tumors), such as Basal Cell Carcinoma (BCC) and medulloblastoma. In BCC and some medulloblastomas, malignant activation of the Hh pathway is due, at least in part, to a genetic mutation in the Patched receptor Inhibition of the Smoothened receptor is believed to disrupt the malignant activation of the Hh pathway by ensuring that Gli transcription factors are held in an inactive form. Other examples of tumor cell inhibition include ligand-dependent sarcomas where inhibition of Hh signaling is believed to inhibit autologous signaling. Chondrosarcomas provide an example of such tumors. In other embodiments, Hh inhibitors can target the tumor microenvironment of ligand dependent cancers, such as desmoplastic tumors, e.g., pancreatic cancer and/or neurodendocrine tumors). In such embodiments, hedgehog inhibitors can decrease fibrosis, thus leading to improved drug delivery. In other embodiments, hedgehog inhibitors can target ligand-dependent residual disease, for example, in solid tumors and heme malignancies. Examples of these solid tumors include SCLC, ovarian and bladder cancer; exemplary heme malignancies include CML, CLL, ALL and AML.

Applicants have discovered, at least in part, that responsiveness to a hedgehog signal (e.g., a hedgehog ligand) in a cancer or tumor cell can be correlated with the presence of cilium in the cancer or tumor cell. In one embodiment, several tumor cell lines were tested for Hh pathway responsiveness and for the presence or absence of cilia or vimentin immunofluorescence (IF). Hh pathway responsiveness was evaluated by detecting increases in hGli-1 mRNA expression in response to Sonic Hedgehog stimulation. A correlation between the presence of cilia in several tumor cell lines and hedgehog signaling capability was established. Similar results were found in Basal Cell Carcinoma (BCC) tissue samples.

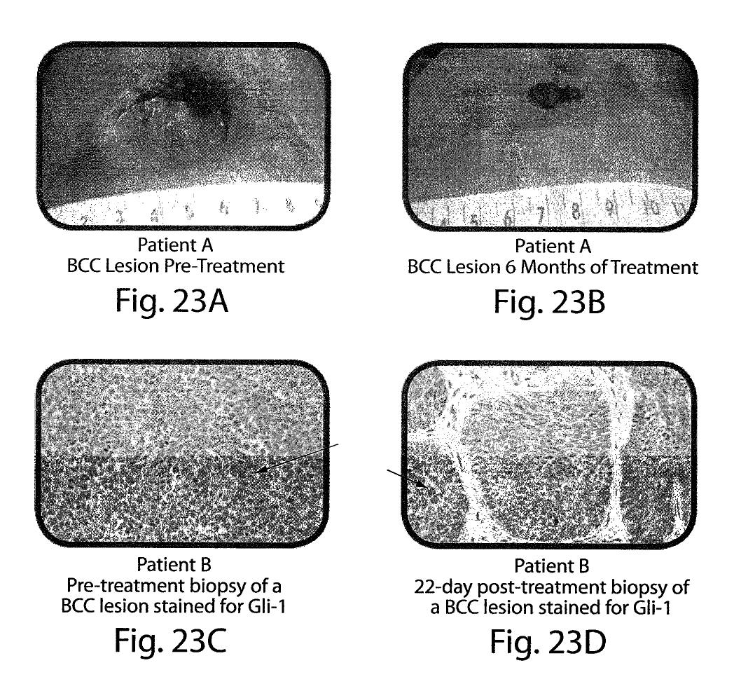

It has been further discovered that increased nuclear Gli1 immunostaining is associated with increased Hh responsiveness, and that inhibition pf Hh nuclear staining is associated with positive clinical activity to Hh inhibition (FIGS. 23C-23D).

In certain embodiments, an alteration in desmoplasia in the tumor sample can be used as a maker to evaluate responsiveness to Hh inhibition. For example, a change in the level of one or more of: collagen, fibronectin or alpha-smooth muscle-specific actin (SMA) levels can be detected.

In other embodiment, the hedgehog-associated biomarker evaluated can be a change in the level of a pericytic marker. The pericytic marker can be chosen from one or more of: NG-2 (CSPG4), RGS5, sphingosine-1-phosphate (S-1-P), PDGF-BB, N-cadherin, or PDGFR-beta. An increase in the level of a pericytic marker, such as S-1-P and/or PDGF-BB can be indicative of increased responsiveness to a hedgehog inhibitor.

In other embodiments, the hedgehog-associated biomarker evaluated can be a change in the level of a gene according to Table 2 differentially expressed in chondrosarcoma.

Thus, an alteration in a hedgehog biomarker as described herein in a tumor or cancer sample can serve as a predictive biomarker of hedgehog responsiveness, i.e., to distinguish patients who will benefit from those who will not benefit from treatment to a hedgehog inhibitor. Detection of these biomarkers can also provide useful diagnostic and prognostic reagents for identifying a subject (e.g., a cancer patient) in need of hedgehog therapy, for example, a subject in need of therapy with a hedgehog inhibitor, such as IPI-926.

In other embodiments, Applicants have discovered that IPI-926 enhances chemotherapeutic drug delivery to tumors by influencing the hedgehog signaling pathway in perivascular fibroblasts, such as pericytes. Without being bound by theory, inhibition of hedgehog signaling in perivascular fibroblasts associated with a tumor or cancer cell can reduce interstitial fluid pressure in the tumor or cancer cell, thereby enhancing delivery of a therapeutic agent to the tumor or cancer cell. Alternatively, hedgehog inhibition and/or anti-cancer therapy can be enhanced by selective targeting one or more hedgehog inhibitor(s) and/or therapeutic agents to perivascular fibroblasts. Thus, targeting perivascular fibroblasts, e.g., pericytes, is a viable strategy to enhance the delivery and efficacy of hedgehog inhibition and/or chemotherapeutic agents.

The present invention provides methods, assays and kits for evaluating a sample, e.g., a sample from a cancer patient, to detect a hedgehog-associated biomarker. In one embodiment, the hedgehog-associated marker evaluated is one or more of: Gli1, e.g., nuclear Gli1; the presence or absence of primary cilia; an alteration in desmoplasia; or an alteration in a pericytic marker. For example, the presence of primary cilia and/or an increased level of nuclear Gli1 (including one or more of Gli1 expression, subcellular localization, or stability) in a tumor or cancer can serve as a predictive biomarker of hedgehog responsiveness. Detection of a hedgehog-associated marker can provide a useful diagnostic, predictive, and prognostic reagent for identifying a subject (e.g., a cancer patient) in need of therapy, or likely to be responsive to therapy, with a hedgehog inhibitor, such as IPI-926. Thus, the methods, assays and kits of the invention can be used, inter alia, as a means to identify patients likely to benefit from administration of a hedgehog inhibitor, alone or in combination with a cancer therapy (e.g., one or more therapeutic agents, radiation and/or surgery); to predict a time course of disease or a probability of a significant event in the disease of a cancer patient; to stratify patient populations; and/or to more effectively treat or prevent a cancer or a tumor associated with hedgehog signaling.

As used herein, a “hedgehog-associated biomarker” or a “marker” as generally referred throughout, includes any detectable indication of hedgehog signaling, including a gene or a gene product (e.g., DNA, RNA, protein), a change in tumor architecture, vascularity, stromal content, desmoplasia, tumor perfusion, pericyte activity and/or distribution, including but not limited to: (i) Gli1, e.g., nuclear Gli1; (ii) cilium or a component thereof; (iii) an upstream or downstream component(s) of the hedgehog signaling pathway, or the signaling pathway of a second agent used in combination with a hedgehog inhibitor (e.g., Gemcitabine); (iv) a genomic marker associated with a cancer, the hedgehog signaling pathway, or the signaling pathway of a second agent used in combination with a hedgehog inhibitor; (v) a marker of Epithelial to Mesenchymal Transition (EMT) associated with a cancer, the hedgehog signaling pathway, or the signaling pathway of a second agent used in combination with a hedgehog inhibitor; (vi) a Gemcitabine marker used in combination with a hedgehog inhibitor, and/or (vii) tumor architecture associated with a cancer, the hedgehog signaling pathway, or the signaling pathway of a second agent used in combination with the hedgehog inhibitor.

A “component” of the hedgehog signaling pathway includes an upstream or downstream component(s) of the hedgehog signaling pathway, e.g., a hedgehog receptor (e.g., patched (PTCH) or smoothened (SMO)), an activator or inhibitor of hedgehog, or a signaling mediator (e.g., Gli1, Gli2, and Gli3).

Primary cilia appear to be required for hedgehog (Hh) signaling. Mutations that affect the assembly or maintenance of cilia have been shown to cause defects in activation of the Hh pathway. For example, Huangfu, D. et al. (2003) Nature 426:83-87 have shown that mutations in intraflagellar transport proteins (IFTs) and KiF3a abrogated hedgehog signaling and resulted in loss of ventral neural cell types. Hedgehog pathway activation depends on the proper localization of hedgehog signaling components, for example, dynamic movement of the hedgehog receptors, patched (Ptch) and smoothened (Smo) into and out of the cilium, activation of Gli1 and/or Gli-2, and processing of Gli3 from activator to repressor (Zaghloul (2009) J. Clin Inv. 119(3): 428-437). Some reports have shown the selective translocation of intracellular Smo to the primary cilium in response to modulation of the hedgehog pathway. Some hedgehog inhibitors, e.g., GDC-0449, are believed to reduce or block movement of Smo to the cilium. Other hedgehog inhibitors can reduce hedgehog signaling, while allowing (or even promoting)

Smo to localize to the cilium. For example, some reports have shown that cyclopamine promotes Smo accumulation at the primary cilium.

As used herein, a “cilium marker” includes cilium (e.g., primary cilium) or a component thereof. Examples of cilia and components thereof include but are not limited to, a microtubule or a component thereof (e.g., tubulin), a component of intraflagellar transport (IFT), a kinesin (e.g., kinesin II), a microtubule organizing center or a component thereof, a basal body or a component thereof (e.g., basal body proteins such as CEP164, ODF2 and CEP170). The term “cilium marker” additionally includes a phosphorylated component of the cilia, e.g., a phosphorylated hegdehog receptor (e.g., a phosphorylated smoothened receptor (Smo)).

Various aspects of the invention are described in further detail in the following subsections.

### DEFINITIONS

As used herein, each of the following terms has the meaning associated with it in this section.

Definitions of specific functional groups and chemical terms are described in detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in, for example, Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, i.e., stereoisomers (enantiomers, diastereomers, cis-trans isomers, E/Z isomers, etc.). Thus, inventive compounds and pharmaceutical compositions thereof can be in the form of an individual enantiomer, diastereomer or other geometric isomer, or can be in the form of a mixture of stereoisomers. Enantiomers, diastereomers and other geometric isomers can be isolated from mixtures (including racemic mixtures) by any method known to those skilled in the art, including chiral high pressure liquid chromatography (HPLC) and the formation and crystallization of chiral salts or prepared by asymmetric syntheses; see, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Carbon atoms, unless otherwise specified, can optionally be substituted with one or more substituents. The number of substituents is typically limited by the number of available valences on the carbon atom, and can be substituted by replacement of one or more of the hydrogen atoms that would be available on the unsubstituted group. Suitable substituents are known in the art and include, but are not limited to, alkyl, alkenyl, alkynyl, alkoxy, alkoxy, aryl, aryloxy, arylthio, aralkyl, heteroaryl, heteroaralkyl, cycloalkyl, heterocyclyl, halo, azido, hydroxyl, thio, alkthiooxy, amino, nitro, nitrile, imino, amido, carboxylic acid, aldehyde, carbonyl, ester, silyl, alkylthio, haloalkyl (e.g., perfluoroalkyl such as —CF₃), ═O, ═S, and the like.

When a range of values is listed, it is intended to encompass each value and sub-range within the range. For example, an alkyl group containing 1-6 carbon atoms (C₁₋₆ alkyl) is intended to encompass, C₁, C₂, C₃, C₄, Cₛ, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

The term “alkyl,” as used herein, refers to saturated, straight- or branched-chain hydrocarbon radical containing between one and thirty carbon atoms. In certain embodiments, the alkyl group contains 1-20 carbon atoms. Alkyl groups, unless otherwise specified, can optionally be substituted with one or more substituents. In certain embodiments, the alkyl group contains 1-10 carbon atoms. In certain embodiments, the alkyl group contains 1-6 carbon atoms. In certain embodiments, the alkyl group contains 1-5 carbon atoms. In certain embodiments, the alkyl group contains 1-4 carbon atoms. In certain embodiments, the alkyl group contains 1-3 carbon atoms. In certain embodiments, the alkyl group contains 1-2 carbon atoms. In certain embodiments, the alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a straight- or branched-chain hydrocarbon radical having at least one carbon-carbon double bond by the removal of a single hydrogen atom, and containing between two and thirty carbon atoms. Alkenyl groups, unless otherwise specified, can optionally be substituted with one or more substituents. In certain embodiments, the alkenyl group contains 2-20 carbon atoms. In certain embodiments, the alkenyl group contains 2-10 carbon atoms. In certain embodiments, the alkenyl group contains 2-6 carbon atoms. In certain embodiments, the alkenyl group contains 2-5 carbon atoms. In certain embodiments, the alkenyl group contains 2-4 carbon atoms. In certain embodiment, the alkenyl group contains 2-3 carbon atoms. In certain embodiments, the alkenyl group contains 2 carbon atoms. Alkenyl groups include, for example, ethenyl, propenyl, butenyl, 1-methyl-2-buten-1-yl, and the like.

The term “alkynyl,” as used herein, denotes a straight- or branched-chain hydrocarbon radical having at least one carbon-carbon triple bond by the removal of a single hydrogen atom, and containing between two and thirty carbon atoms. Alkynyl groups, unless otherwise specified, can optionally be substituted with one or more substituents. In certain embodiments, the alkynyl group contains 2-20 carbon atoms. In certain embodiments, the alkynyl group contains 2-10 carbon atoms. In certain embodiments, the alkynyl group contains 2-6 carbon atoms. In certain embodiments, the alkynyl group contains 2-5 carbon atoms. In certain embodiments, the alkynyl group contains 2-4 carbon atoms. In certain embodiments, the alkynyl group contains 2-3 carbon atoms. In certain embodiments, the alkynyl group contains 2 carbon atoms. Representative alkynyl groups include, but are not limited to, ethynyl, 2-propynyl (propargyl), 1-propynyl, and the like.

The terms “cycloalkyl”, used alone or as part of a larger moiety, refer to a saturated monocyclic or bicyclic hydrocarbon ring system having from 3-15 carbon ring members. Cycloalkyl groups, unless otherwise specified, can optionally be substituted with one or more substituents. In certain embodiments, cycloalkyl groups contain 3-10 carbon ring members. In certain embodiments, cycloalkyl groups contain 3-9 carbon ring members. In certain embodiments, cycloalkyl groups contain 3-8 carbon ring members. In certain embodiments, cycloalkyl groups contain 3-7 carbon ring members. In certain embodiments, cycloalkyl groups contain 3-6 carbon ring members. In certain embodiments, cycloalkyl groups contain 3-5 carbon ring members. Cycloalkyl groups include, without limitation, cyclopropyl, cyclobutyl, cyclopentyl, cyclohexyl, cycloheptyl, and cyclooctyl. The term “cycloalkyl” also includes saturated hydrocarbon ring systems that are fused to one or more aryl or heteroaryl rings, such as decahydronaphthyl or tetrahydronaphthyl, where the point of attachment is on the saturated hydrocarbon ring.

The term “aryl” used alone or as part of a larger moiety (as in “aralkyl”), refers to an aromatic monocyclic and bicyclic hydrocarbon ring system having a total of 6-10 carbon ring members. Aryl groups, unless otherwise specified, can optionally be substituted with one or more substituents. In certain embodiments of the present invention, “aryl” refers to an aromatic ring system which includes, but not limited to, phenyl, biphenyl, naphthyl, anthrancyl and the like, which can bear one or more substituents. Also included within the scope of the term “aryl”, as it is used herein, is a group in which an aryl ring is fused to one or more non-aromatic rings, such as indanyl, phthalimidyl or tetrahydronaphthalyl, and the like, where the point of attachment is on the aryl ring.

The term “aralkyl” refers to an alkyl group, as defined herein, substituted by aryl group, as defined herein, wherein the point of attachment is on the alkyl group.

The term “heteroatom” refers to boron, phosphorus, selenium, nitrogen, oxygen, or sulfur, and includes any oxidized form of nitrogen or sulfur, and any quaternized form of abasic nitrogen.

The terms “heteroaryl” used alone or as part of a larger moiety, e.g., “heteroaralkyl”, refer to an aromatic monocyclic or bicyclic hydrocarbon ring system having 5-10 ring atoms wherein the ring atoms comprise, in addition to carbon atoms, from one to five heteroatoms. Heteroaryl groups, unless otherwise specified, can optionally be substituted with one or more substituents. When used in reference to a ring atom of a heteroaryl group, the term “nitrogen” includes a substituted nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaryl ring is fused to one or more aryl, cycloalkyl or heterocycloalkyl rings, wherein the point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl.

The term “heteroaralkyl” refers to an alkyl group, as defined herein, substituted by a heteroaryl group, as defined herein, wherein the point of attachment is on the alkyl group.

As used herein, the terms “heterocycloalkyl” or “heterocyclyl” refer to a stable non-aromatic 5-7 membered monocyclic hydrocarbon or stable non-aromatic 7-10 membered bicyclic hydrocarbon that is either saturated or partially unsaturated, and having, in addition to carbon atoms, one or more heteroatoms. Heterocycloalkyl or heterocyclyl groups, unless otherwise specified, can optionally be substituted with one or more substituents. When used in reference to a ring atom of a heterocycloalkyl group, the term “nitrogen” includes a substituted nitrogen. The point of attachment of a heterocycloalkyl group can be at any of its heteroatom or carbon ring atoms that results in a stable structure. Examples of heterocycloalkyl groups include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. “Heterocycloalkyl” also include groups in which the heterocycloalkyl ring is fused to one or more aryl, heteroaryl or cycloalkyl rings, such as indolinyl, chromanyl, phenanthridinyl, or tetrahydroquinolinyl, where the radical or point of attachment is on the heterocycloalkyl ring.

The term “unsaturated”, as used herein, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but is not intended to include aromatic groups, such as aryl or heteroaryl moieties, as defined herein.

The term “diradical” as used herein refers to an alkyl, alkenyl, alkynyl, aryl, cycloalkyl, heterocycloalkyl, aralkyl, heteroaryl, and heteroaralkyl groups, as described herein, wherein 2 hydrogen atoms are removed to form a divalent moiety (e.g., an alkyl diradical, an alkenyl diradical, an alkynyl diradical, an aryl diradical, a cycloalkyl diradical, a heterocycloalkyl diradical, an aralkyl diradical, a heteroaryl diradical, and a heteroaralkyl diradical). Diradicals are typically end with a suffix of “-ene”. For example, alkyl diradicals are referred to as alkylenes (for example:

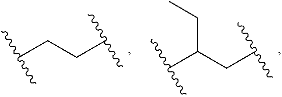

and —(CR′₂)ₓ— wherein R′ is hydrogen or other substituent and x is 1, 2, 3, 4, 5 or 6); alkenyl diradicals are referred to as “alkenylenes”; alkynyl diradicals are referred to as “alkynylenes”; aryl and aralkyl diradicals are referred to as “arylenes” and “aralkylenes”, respectively (for example:

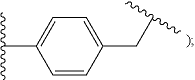

heteroaryl and heteroaralkyl diradicals are referred to as “heteroarylenes” and “heteroaralkylenes”, respectively (for example:

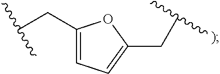

cycloalkyl diradicals are referred to as “cycloalkylenes”; heterocycloalkyl diradicals are referred to as “heterocycloalkylenes”; and the like.

The terms “halo”, “halogen” and “halide” as used herein refer to an atom selected from fluorine (fluoro, F), chlorine (chloro, Cl), bromine (bromo, Br), and iodine (iodo, I).

As used herein, the term “haloalkyl” refers to an alkyl group, as described herein, wherein one or more of the hydrogen atoms of the alkyl group is replaced with one or more halogen atoms. In certain embodiments, the haloalkyl group is a perhaloalkyl group, that is, having all of the hydrogen atoms of the alkyl group replaced with halogens (e.g., such as the perfluoroalkyl group —CF₃).

As used herein, the term “azido” refers to the group —N₃.

As used herein, the term “nitrile” refers to the group —CN.

As used herein, the term “nitro” refers to the group —NO₂.

As used herein, the term “hydroxyl” or “hydroxy” refers to the group —OH.

As used herein, the term “thiol” or “thio” refers to the group —SH.

As used herein, the term “carboxylic acid” refers to the group —CO₂H.

As used herein, the term “aldehyde” refers to the group —CHO.

As used herein, the term “alkoxy” refers to the group —OR′, wherein R′ is an alkyl, alkenyl or alkynyl group, as defined herein.

As used herein, the term “aryloxy” refers to the group —OR′, wherein each R′ is an aryl or heteroaryl group, as defined herein.

As used herein, the term “alkthiooxy” refers to the group —SR′, wherein each R′ is, independently, a carbon moiety, such as, for example, an alkyl, alkenyl, or alkynyl group, as defined herein.

As used herein, the term “arylthio” refers to the group —SR′, wherein each R′ is an aryl or heteroaryl group, as defined herein.

As used herein, the term “amino” refers to the group —NR′₂, wherein each R′ is, independently, hydrogen, a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “carbonyl” refers to the group —C(═O)R′, wherein R′ is, independently, a carbon moiety, such as, for example, an alkyl, alkenyl, alkynyl, aryl or heteroaryl group, as defined herein.

As used herein, the term “ester” refers to the group —C(═O)OR′ or —OC(═O)R′ wherein each R′ is, independently, a carbon moiety, such as, for example, an alkyl, alkenyl, alkynyl, aryl or heteroaryl group, as defined herein.

As used herein, the term “amide” or “amido” refers to the group —C(═O)N(R′)₂ or —NR′C(═O)R′ wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfonamido” or “sulfonamide” refers to the group —N(R′)SO₂R′ or —SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfamido” or “sulfamide” refers to the group —NR′SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “imide” or “imido” refers to the group —C(═NR′)N(R′)₂ or —NR′C(═NR′)R′ wherein each R′ is, independently, hydrogen or a carbon moiety, such as, for example, an alkyl, alkenyl, alkynyl, aryl or heteroaryl group, as defined herein, or wherein two R′ groups together with the nitrogen atom to which they are bound form a 5-8 membered ring.

As used herein “silyl” refers to the group —SiR′ wherein R′ is a carbon moiety, such as, for example, an alkyl, alkenyl, alkynyl, aryl or heteroaryl group.

In some cases, the hedgehog inhibitor can contain one or more basic functional groups (e.g., such as an amino group), and thus is capable of forming pharmaceutically acceptable salts with pharmaceutically acceptable acids. The term “pharmaceutically acceptable salts” in these instances refers to the relatively non-toxic, inorganic and organic acid addition salts. These salts can be prepared in situ in the administration vehicle or the dosage form manufacturing process, or by separately treating the compound in its free base form with a suitable acid. Examples of pharmaceutically acceptable, nontoxic acid addition salts from inorganic acids include, but are not limited to, hydrochloric, hydrobromic, phosphoric, sulfuric, nitric and perchloric acid or from organic acids include, but are not limited to, acetic, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic, and valeric acid addition salts, and the like. In other cases, the hedgehog inhibitor can contain one or more acidic functional groups, and thus is capable of forming pharmaceutically acceptable salts with pharmaceutically acceptable bases. The term “pharmaceutically acceptable salts” in these instances refers to the relatively non-toxic, inorganic and organic base addition salts. These salts can likewise be prepared in situ in the administration vehicle or the dosage form manufacturing process, or by separately treating the compound in its free acid form with a suitable base. Examples of suitable bases include, but are not limited to, metal hydroxides, metal carbonates or metal bicarbonates, wherein the metal is an alkali or alkaline earth metal such as lithium, sodium, potassium, calcium, magnesium, or aluminum. Suitable bases can also include ammonia or organic primary, secondary or tertiary amines. Representative organic amines useful for the formation of base addition salts include, for example, ethylamine, diethylamine, ethylenediamine, ethanolamine, diethanolamine, piperazine and the like (see, e.g., Berge et al., supra).

The term “solvate” refers to a compound of the present invention having either a stoichiometric or non-stoichiometric amount of a solvent associated with the compound. The solvent can be water (i.e., a hydrate), and each molecule of inhibitor can be associated with one or more molecules of water (e.g., monohydrate, dihydrate, trihydrate, etc.). The solvent can also be an alcohol (e.g., methanol, ethanol, propanol, isopropanol, etc.), a glycol (e.g., propylene glycol), an ether (e.g., diethyl ether), an ester (e.g., ethyl acetate), or any other suitable solvent. The hedgehog inhibitor can also exist as a mixed solvate (i.e., associated with two or more different solvents).

The term “sugar” as used herein refers to a natural or an unnatural monosaccharide, disaccharide or oligosaccharide comprising one or more pyranose or furanose rings. The sugar can be covalently bonded to the steroidal alkaloid of the present invention through an ether linkage or through an alkyl linkage. In certain embodiments the saccharide moiety can be covalently bonded to a steroidal alkaloid of the present invention at an anomeric center of a saccharide ring. Sugars can include, but are not limited to ribose, arabinose, xylose, lyxose, allose, altrose, glucose, mannose, gulose, idose, galactose, talose, glucose, and trehalose.

Additional terms are defined herein.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

A “biomarker gene or gene product” or “marker gene or gene product” is a gene, mRNA, or protein which can be altered, wherein said alteration is associated with cancer or in response to chemo- or hedgehog treatment. The alteration can be in amount, level, structure, subcellular localization, and/or activity in a cancer tissue or cancer cell, as compared to a reference value, e.g., its amount, level, structure, and/or activity, in a normal or healthy tissue or cell (e.g., a control). For example, a marker which is associated with cancer or predictive of responsiveness to anti-cancer therapeutics can have an altered nucleotide sequence, amino acid sequence, chromosomal translocation, intra-chromosomal inversion, copy number, expression level, protein level, subcellular localization, protein activity, or methylation status, in a cancer tissue or cancer cell as compared to a normal, healthy tissue or cell. Furthermore, a “marker” includes a molecule whose structure is altered, e.g., mutated (contains an mutation), e.g., differs from the wild type sequence at the nucleotide or amino acid level, e.g., by substitution, deletion, or insertion, when present in a tissue or cell associated with a disease state, such as cancer.

The term “altered amount,” “altered level,” or “altered subcellular localization” of a marker refers to any of: (i) increased or decreased copy number of a marker or chromosomal region, such as gene mutations and/or gene products; (ii) increased or decreased gene expression level of a particular marker gene or genes in a cancer sample; (iii) an increased or decreased protein level of a marker in a sample; (iv) an alteration in subcellular distribution, e.g., increased nuclear localization; as compared to the amount, level, or localization of the marker in a reference, e.g., a control sample. The term “altered level” need not result from increased or decreased expression of a gene or gene product, but differences in, e.g., subcellular localization, can lead to an altered level in a subcellular compartment, e.g., increased nucleus compared to the cytoplasm.

The term “altered level of expression” of an alteration, e.g., gene mutations and/or gene products, or other markers disclosed herein, refers to an expression level or copy number of a marker in a test sample, such as a sample derived from a patient suffering from cancer, that is greater or less than the standard error of the assay employed to assess expression or copy number. In embodiments, the alteration can be at least twice, at least twice three, at least twice four, at least twice five, or at least twice ten or more times the expression level or copy number of the alterations, e.g., gene mutations and/or gene products in a control sample (e.g., a sample from a healthy subject not having the associated disease, or a non-malignant sample from the subject), or the average expression level or copy number of the alterations, e.g., gene mutations and/or gene products in several control samples. The altered level of expression is greater or less than the standard error of the assay employed to assess expression or copy number. In embodiments, the alteration is at least twice, at least three, at least four, at least five, at least ten or more times the expression level or copy number of the alterations, e.g., gene mutations and/or gene products in a control sample (e.g., a sample from a healthy subject not having the associated disease), or the average expression level or copy number of the alterations, e.g., gene mutations and/or gene products (in several control samples.

The term “altered activity” of a marker refers to an activity of a marker which is increased or decreased in a disease state, e.g., in a cancer sample, as compared to the activity of the marker in a normal, control sample. Altered activity of a marker can be the result of, for example, altered expression of the marker, altered protein level of the marker, altered structure of the marker, or, e.g., an altered interaction with other proteins involved in the same or different pathway as the marker.

The term “altered structure” or “alteration” of a marker, gene or gene product refers to the presence of mutations or mutations within the marker gene or maker protein, e.g., mutations which affect expression or activity of the marker, as compared to the normal or wild-type gene or protein. For example, mutations include, but are not limited to inter- and intra-chromosomal rearrangement, substitutions, deletions, and insertion mutations. Mutations can be present in the coding or non-coding region of the marker.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an anti-parallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

The “copy number of a gene” or the “copy number of a marker” refers to the number of DNA sequences in a cell encoding a particular gene product. Generally, for a given gene, a mammal has two copies of each gene. The copy number can be increased, however, by gene amplification or duplication, or reduced by deletion.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, at least 60%, at least 65%, at least 70%, at least 75%, at least 80%, at least 85%, at least 90%, at least 95% or more.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

A “transcribed polynucleotide” is a polynucleotide (e.g., an RNA, a cDNA, or an analog of one of an RNA or cDNA) which is complementary to or homologous with all or a portion of a mature RNA made by transcription of a marker of the invention and normal post-transcriptional processing (e.g., splicing), if any, of the transcript, and reverse transcription of the transcript.

The “normal” copy number of a marker or “normal” level of expression of a marker is the level of expression, copy number of the marker, in a biological sample, e.g., a sample containing tissue, whole blood, serum, plasma, buccal scrape, saliva, cerebrospinal fluid, urine, stool, and bone marrow, from a subject, e.g., a human, not afflicted with cancer.

An “overexpression” or “significantly higher level of expression or copy number” of the gene mutations and/or gene products refers to an expression level or copy number in a test sample that is greater than the standard error of the assay employed to assess expression or copy number. In embodiments, the overexpression can be at least two, at least three, at least four, at least five, or at least ten or more times the expression level or copy number of the gene mutations and/or gene products in a control sample, or the average expression level or copy number of the gene mutations and/or gene products in several control samples.

An “underexpression” or “significantly lower level of expression or copy number” of gene mutations and/or gene products refers to an expression level or copy number in a test sample that is greater than the standard error of the assay employed to assess expression or copy number, for example, at least twice, at least three, at least four, at least five, or at least ten or more times less than the expression level or copy number of the gene mutations and/or gene products in a control sample (e.g., a sample from a healthy subject not afflicted with cancer, or a non-malignant sample from the patient), or the average expression level or copy number of the gene mutations and/or gene products in several control samples.

The amount of a marker, e.g., expression or copy number of gene mutations and/or gene products, in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about two, at least about three, at least about four, or at least about five times, higher or lower, respectively, than the normal amount of the marker.

As used herein, “cancer” and “tumor” are synonymous terms. The term “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. Cancer cells also include cancer stem cells (CSC). As used herein, the term “cancer” includes premalignant as well as malignant cancers.

As used herein, “cancer therapy” and “cancer treatment” are synonymous terms.

As used herein “therapeutic agent” and “drug” are synonymous terms and are meant to include both biotherapeutic agents (e.g., cancer biologics) as well as chemotherapeutic agents.

As used herein, and unless otherwise specified, the terms “treat,” “treating” and “treatment” contemplate an action that occurs while a patient is suffering from cancer, which reduces the severity of the cancer, or retards or slows the progression of the cancer. Treatment can include, but is not limited to, inhibiting tumor growth, reducing tumor mass, reducing size or number of metastatic lesions, inhibiting the development of new metastatic lesions, prolonged survival, prolonged progression-free survival, prolonged time to progression, and/or enhanced quality of life.

As used herein, unless otherwise specified, the terms “prevent,” “preventing” and “prevention” contemplate an action that occurs before a patient begins to suffer from the re-growth of the cancer and/or which inhibits or reduces the severity of the cancer.

As used herein, and unless otherwise specified, the terms “manage,” “managing” and “management” encompass preventing the recurrence of the cancer in a patient who has already suffered from the cancer, and/or lengthening the time that a patient who has suffered from the cancer remains in remission. The terms encompass modulating the threshold, development and/or duration of the cancer, or changing the way that a patient responds to the cancer.

As used herein, and unless otherwise specified, a “therapeutically effective amount” of a compound is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent.

As used herein, and unless otherwise specified, a “prophylactically effective amount” of a compound is an amount sufficient to prevent regrowth of the cancer, or one or more symptoms associated with the cancer, or prevent its recurrence. A prophylactically effective amount of a compound means an amount of the compound, alone or in combination with other therapeutic agents, which provides a prophylactic benefit in the prevention of the cancer. The term “prophylactically effective amount” can encompass an amount that improves overall prophylaxis or enhances the prophylactic efficacy of another prophylactic agent.

The term “subject” as used herein, refers to an animal, typically a human (i.e., a male or female of any age group, e.g., a pediatric subject (e.g, infant, child, adolescent) or adult subject (e.g., young adult, middle-aged adult or senior adult) or other mammal, such as primates (e.g., cynomolgus monkeys, rhesus monkeys); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the subject has been the object of treatment, observation, and/or administration of the compound or drug.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

“Likely to” or “increased likelihood,” as used herein, refers to an increased probability that an item, object, thing or person will occur. Thus, in one example, a subject that is likely to respond to treatment with a hedgehog inhibiting agent has an increased probability of responding to treatment with an hedgehog inhibiting agent relative to a reference subject or group of subjects.

“Unlikely to” refers to a decreased probability that an event, item, object, thing or person will occur with respect to a reference. Thus, a subject that is unlikely to respond to treatment with a hedgehog inhibiting agent has a decreased probability of responding to treatment with an hedgehog inhibiting agent relative to a reference subject or group of subjects.

“RECIST” shall mean an acronym that stands for “Response Evaluation Criteria in Solid Tumours” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “PR,” “CR,” “SD” and “PD.”

“Responsiveness,” to “respond” to treatment, and other forms of this verb, as used herein, refer to the reaction of a subject to treatment with a hedgehog inhibiting agent. As an example, a subject responds to treatment with a hedgehog inhibiting agent if growth of a tumor in the subject is retarded about 10%, 20%, 30%, 40%, 50%, 60%, 70%, 80%, 90% or more. In another example, a subject responds to treatment with an hedgehog inhibiting agent if a tumor in the subject shrinks by about 5%, 10%, 20%, 30%, 40%, 50% or more as determined by any appropriate measure, e.g., by mass or volume. In another example, a subject responds to treatment with a hedgehog inhibitor if the subject experiences a life expectancy extended by about 5%, 10%, 20%, 30%, 40%, 50% or more beyond the life expectancy predicted if no treatment is administered. In another example, a subject responds to treatment with an hedgehog inhibiting agent if the subject has an increased disease-free survival, overall survival or increased time to progression. Several methods can be used to determine if a patient responds to a treatment including the RECIST criteria, as set forth above.

“Sample,” “tissue sample,” “patient sample,” “patient cell or tissue sample” or “specimen” each refers to a collection of similar cells obtained from a tissue of a subject or patient. The source of the tissue sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like.

As used herein, “significant event” shall refer to an event in a patient's disease that is important as determined by one skilled in the art. Examples of significant events include, for example, without limitation, primary diagnosis, death, recurrence, the determination that a patient's disease is metastatic, relapse of a patient's disease or the progression of a patient's disease from any one of the above noted stages to another. A significant event can be any important event used to assess OS, TTP and/or using the RECIST or other response criteria, as determined by one skilled in the art.

As used herein, “time course” shall refer to the amount of time between an initial event and a subsequent event. For example, with respect to a patient's cancer, time course can relate to a patient's disease and can be measured by gauging significant events in the course of the disease, wherein the first event can be diagnosis and the subsequent event can be metastasis, for example.

“Time to progression” or “TTP” refers to a time as measured from the start of the treatment to progression or a cancer or censor. Censoring can come from a study end or from a change in treatment. Time to progression can also be represented as a probability as, for example, in a Kaplein-Meier plot where time to progression can represent the probability of being progression free over a particular time, that time being the time between the start of the treatment to progression or censor.

Various aspects of the invention are described in further detail below. Additional definitions are set out throughout the specification.

Hedgehog Inhibitors

Suitable hedgehog inhibitors for use with the present invention include, for example, those described and disclosed in U.S. Pat. No. 7,230,004, U.S. patent application Publication No. 2008/0293754, U.S. patent application Publication No. 2008/0287420, and U.S. patent application Publication No. 2008/0293755, the entire disclosures of which are incorporated by reference herein.

Examples of other suitable hedgehog inhibitors include those described in U.S. patent application Publication Nos. US 2002/0006931, US 2007/0021493 and US 2007/0060546, and International application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

Additional examples of hedgehog inhibitors include, but are not limited to, GDC-0449 (also known as RG3616 or vismodegib) described in, e.g., Von Hoff D. et al., N. Engl. J. Med. 2009; 361(12):1164-72; Robarge K. D. et al., Bioorg Med Chem. Lett. 2009; 19(19):5576-81; Yauch, R. L. et al. (2009) Science 326: 572-574; Sciencexpress: 1-3 (10.1126/science.1179386); Rudin, C. et al. (2009) New England J of Medicine 361-366 (10.1056/nejma0902903); BMS-833923 (also known as XL139) described in, e.g., in Siu L. et al., J. Clin. Oncol. 2010; 28:15s (suppl; abstr 2501); and National Institute of Health Clinical Trial Identifier No. NCT006701891; LDE-225 described, e.g., in Pan S. et al., ACS Med. Chem. Lett., 2010; 1(3): 130-134; LEQ-506 described, e.g., in National Institute of Health Clinical Trial Identifier No. NCT01106508; PF-04449913 described, e.g., in National Institute of Health Clinical Trial Identifier No. NCT00953758; Hedgehog pathway antagonists disclosed in U.S. patent application Publication No. 2010/0286114; SMOi2-17 described, e.g., U.S. patent application Publication No. 2010/0093625; SANT-1 and SANT-2 described, e.g., in Rominger C. M. et al., J. Pharmacol. Exp. Ther. 2009; 329(3):995-1005; 1-piperazinyl-4-arylphthalazines or analogues thereof, described in Lucas B. S. et al., Bioorg. Med. Chem. Lett. 2010; 20(12):3618-22.

In certain embodiments, the hedgehog inhibitor is a compound of formula (I):

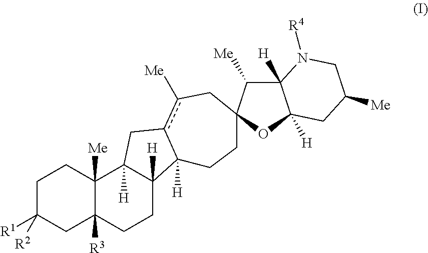

or a pharmaceutically acceptable form thereof (e.g., a salt and/or solvate) thereof; wherein:

R¹ is H, alkyl, —OR, amino, sulfonamido, sulfamido, —OC(O)R⁵, —N(R⁵)C(O)R⁵, or a sugar;

R² is H, alkyl, alkenyl, alkynyl, aryl, cycloalkyl, nitrile, or heterocycloalkyl;

or R¹ and R² taken together form ═O, ═S, ═N(OR), ═N(R), ═N(NR₂), or ═C(R)₂;

R³ is H, alkyl, alkenyl, or alkynyl;

R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, —CO₂R⁵, —SO₂R⁵, —C(O)N(R⁵)(R⁵), —[C(R)₂]_{q}—R⁵, —[(W)—N(R)C(O)]_{q}R⁵, —[(W)—C(O)]_{q}R⁵, —[(W)—C(O)O]_{q}R⁵, —[(W)—OC(O)]_{q}R⁵, —[(W)—SO₂]_{q}R⁵, —[(W)—N(R⁵)SO₂]_{q}R⁵, —[(W)—C(O)N(R⁵)]_{q}R⁵, —[(W)—O]_{q}R⁵, —[(W)—N(R)]_{q}R⁵, —W—NR₃⁺X⁻ or —[(W)—S]_{q}R⁵; wherein each W is independently for each occurrence a diradical such as an alkylene; each q is independently for each occurrence 1, 2, 3, 4, 5, or 6; and X⁻ is an anion (e.g., a halide);

each R⁵ is independently for each occurrence H, alkyl, alkenyl, alkynyl, aryl, cycloalkyl, heterocycloalkyl, aralkyl, heteroaryl, heteroaralkyl or —[C(R)₂]ₚ—R⁶; wherein p is 0-6; or any two occurrences of R⁵ on the same substituent can be taken together to form a 4-8 membered optionally substituted ring which contains 0-3 heteroatoms selected from N, O, S, and P; and

each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR); and

each R is independently H, alkyl, alkenyl, alkynyl, aryl, cycloalkyl or aralkyl;

provided that when R², R³ are H and R⁴ is hydroxyl; R¹ cannot be hydroxyl;

provided that when R², R³, and R⁴ are H; R¹ cannot be hydroxyl; and

provided that when R², R³, and R⁴ are H; R¹ cannot be sugar.

In certain embodiments, R¹ is H, hydroxyl, alkoxyl, aryloxy, or amino.

In some embodiments, R¹ and R² taken together along with the carbon to which they are bonded, form ═O, ═N(OR), or ═S.

In other embodiments, R³ is H and/or R⁴ is H, alkyl, hydroxyl, aralkyl, —[C(R)₂]_{q}—R⁵, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is H or —OR, R² is H or alkyl, and R⁴ is H.

In yet other embodiments, R² is H or alkyl, R³ is H, alkyl, alkenyl, alkynyl, aryl, cycloalkyl, heterocycloalkyl, or aralkyl; and/or R⁴ is H, alkyl, aralkyl, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is sulfonamido.

Specific examples of hedgehog inhibitors include compounds, or pharmaceutically acceptable salts and/or solvates thereof, described in U.S. patent application 2008/0293754 and also provided below in Table 1:

TABLE 1
1

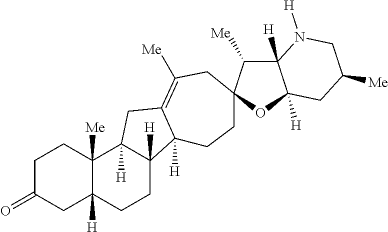

2

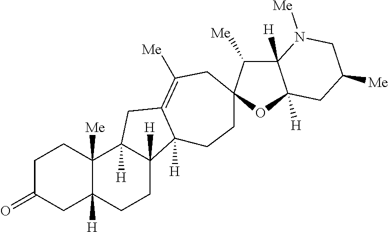

3

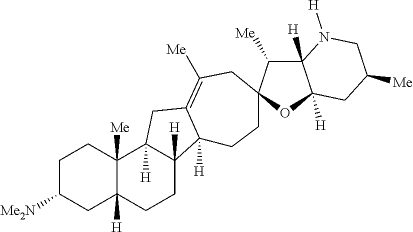

4

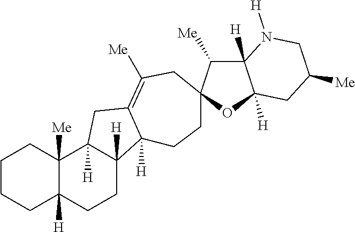

5

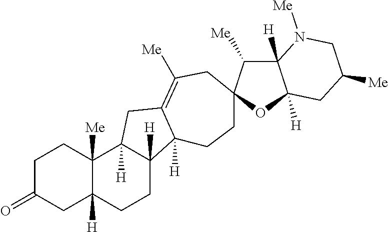

6

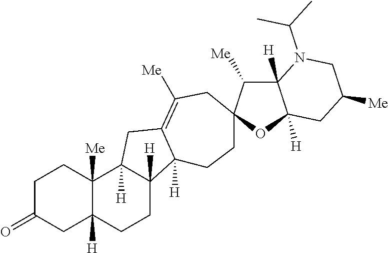

7

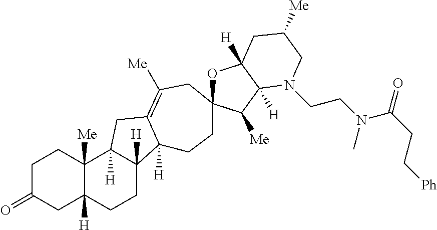

8

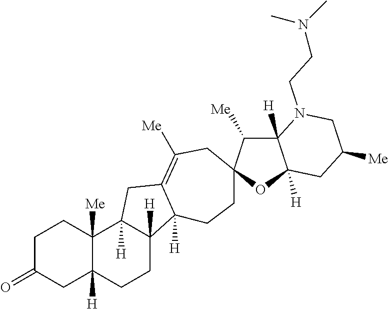

9

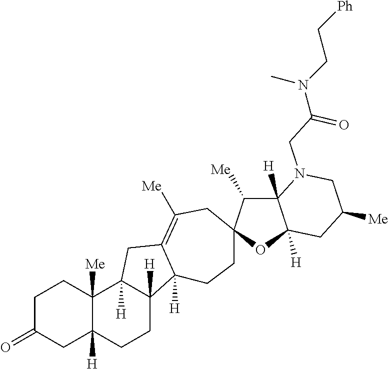

10

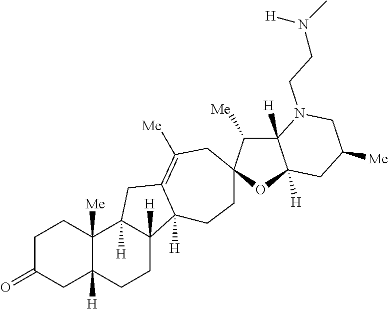

11

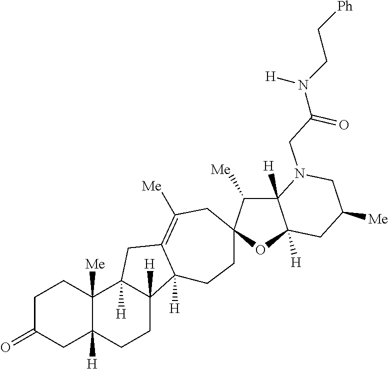

12

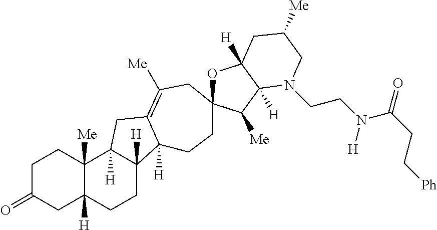

13

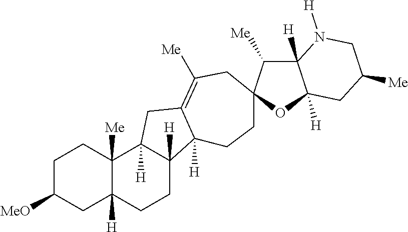

14

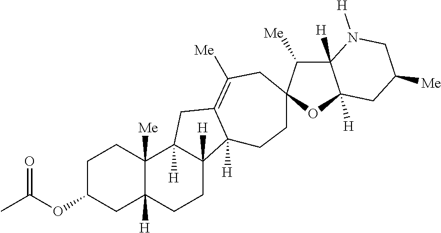

15

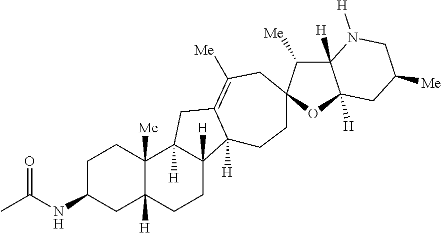

16

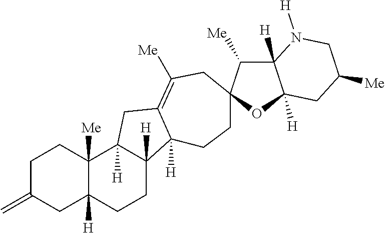

17

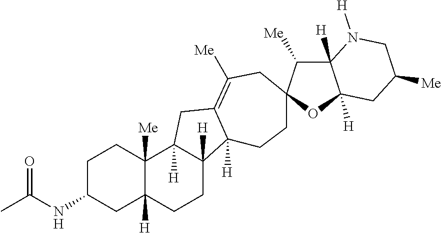

18

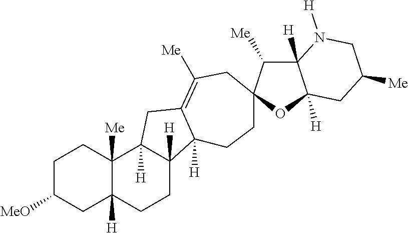

19

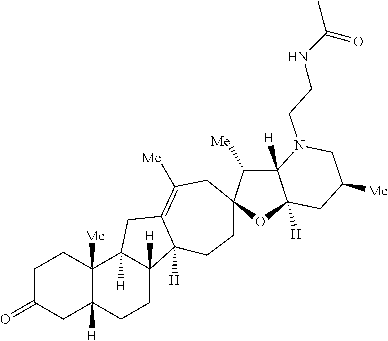

20

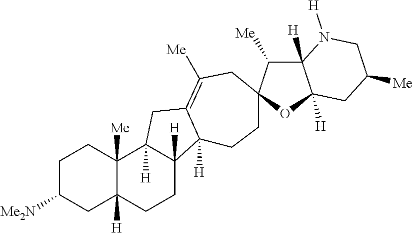

21

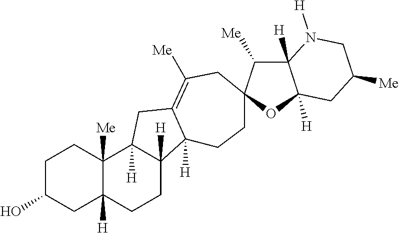

22

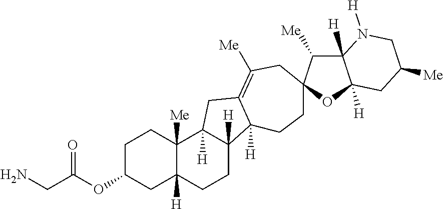

23

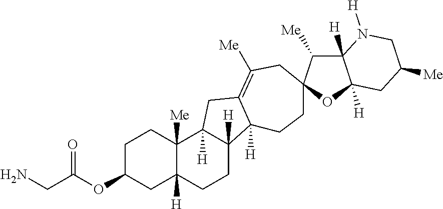

24

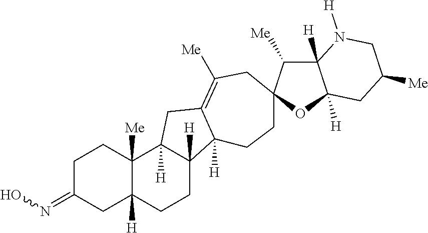

25

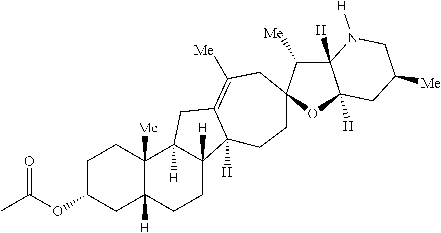

26

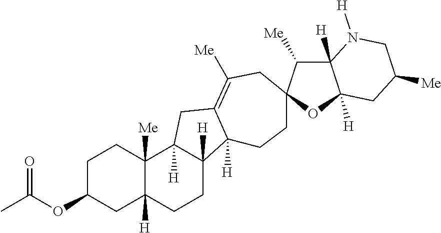

27

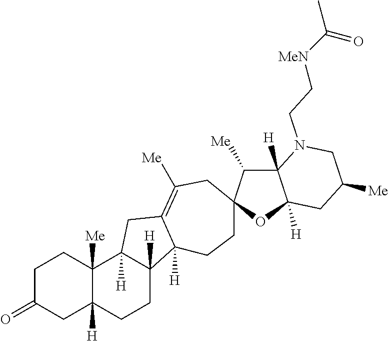

28

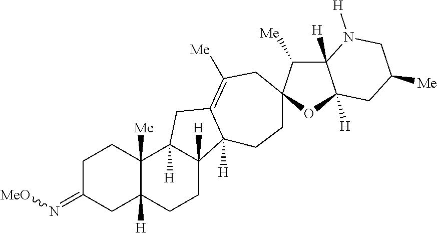

29

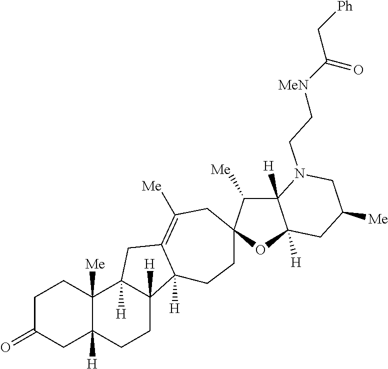

30

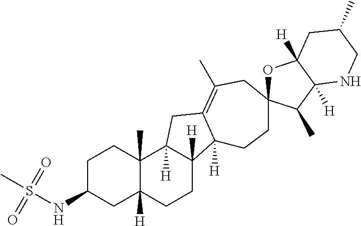

31

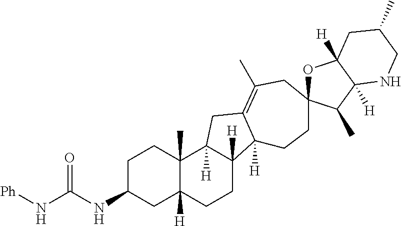

32

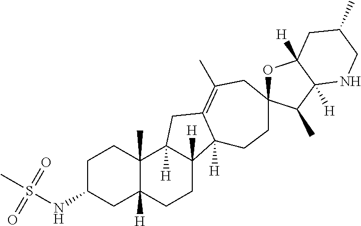

33

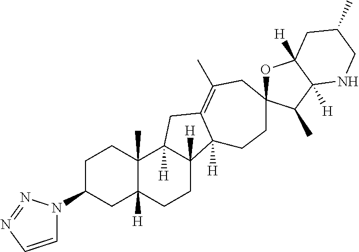

34

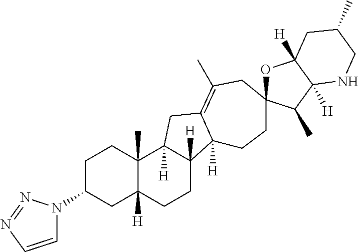

35

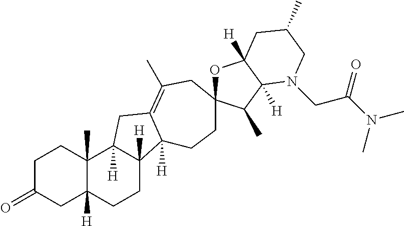

36

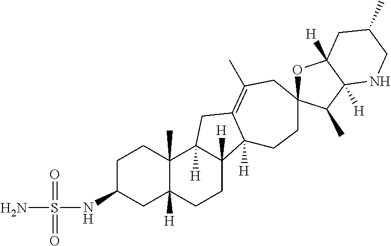

37

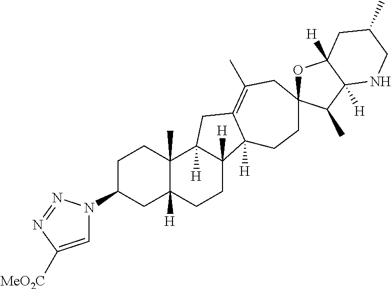

38

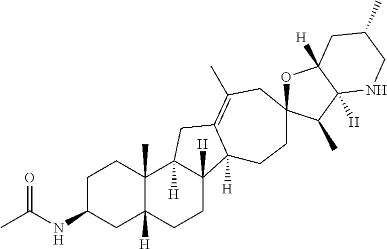

39

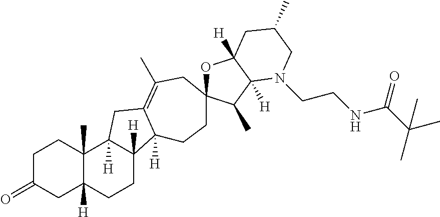

40

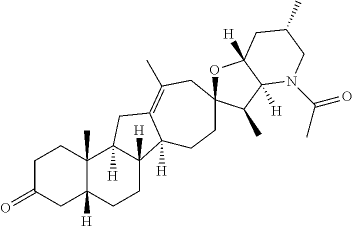

41

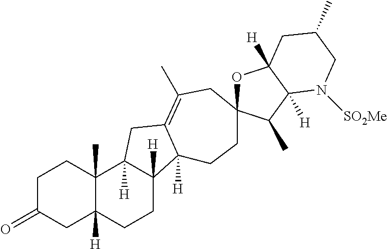

42

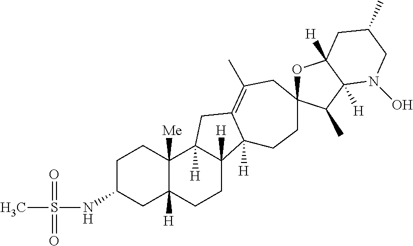

43

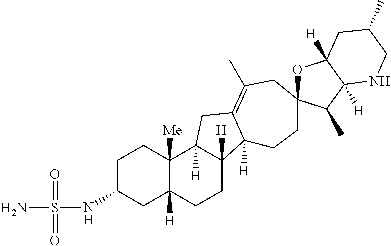

44

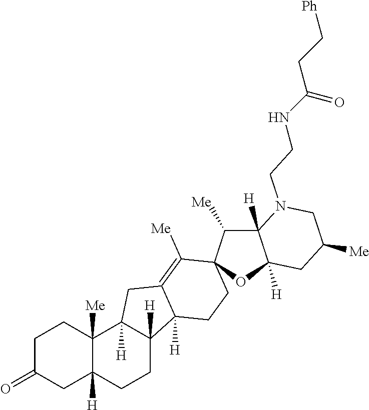

45

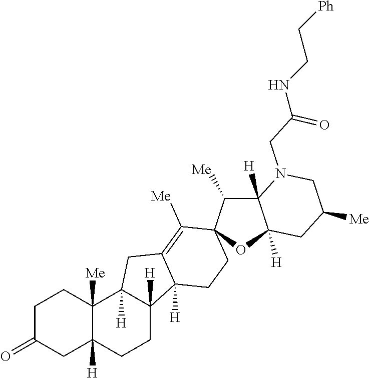

46

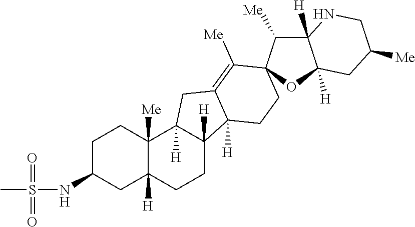

47

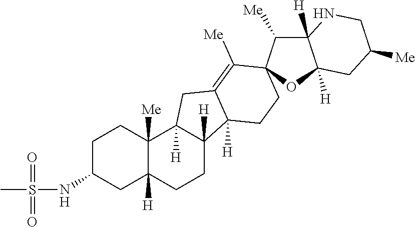

48

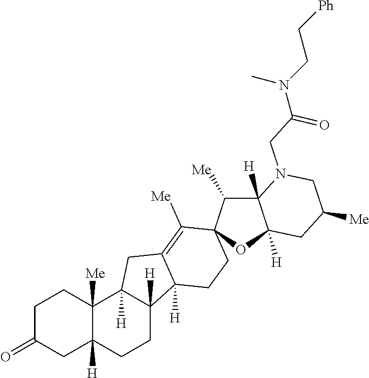

Other examples of hedgehog inhibitors include compounds, or pharmaceutically acceptable salts and/or solvates thereof, described in U.S. Pat. No. 7,230,004 and also provided below in Table 2:

TABLE 2
49

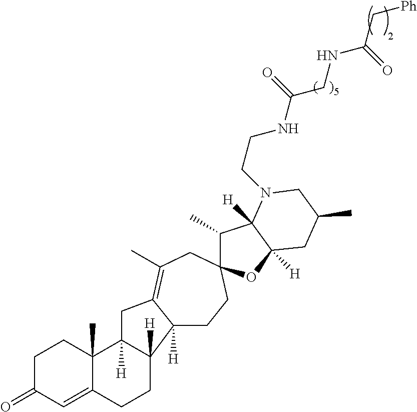

50

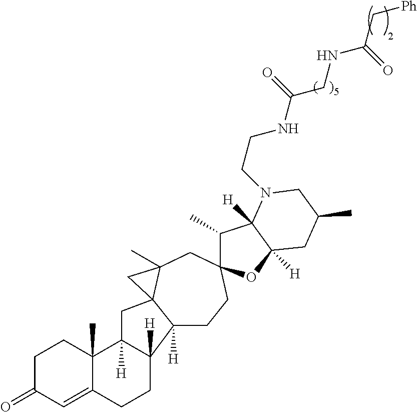

51

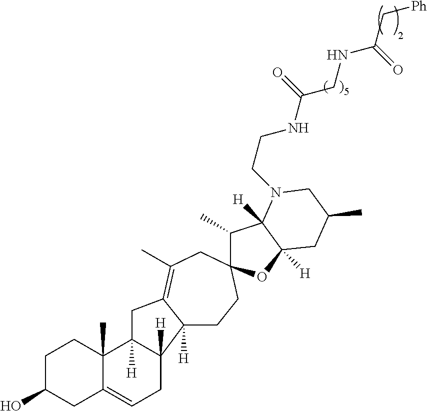

52

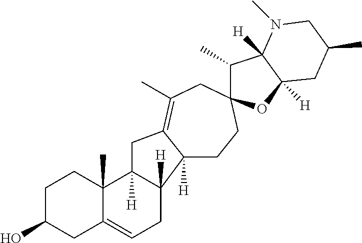

53

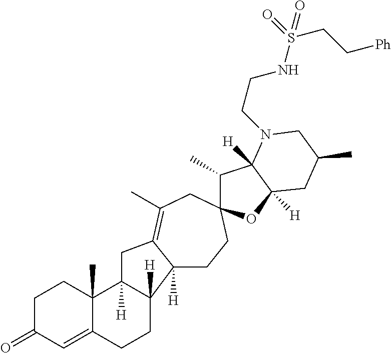

54

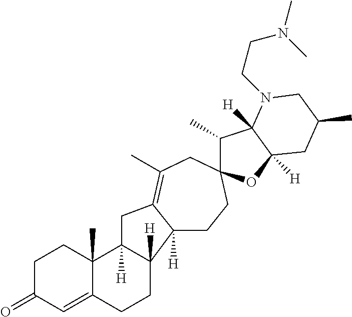

55

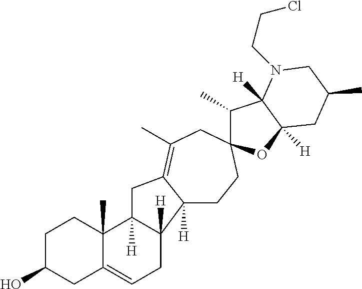

56

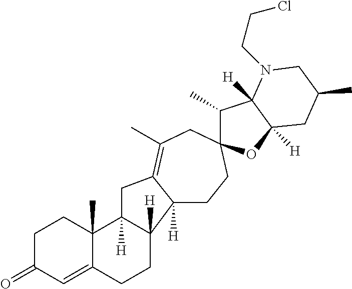

57

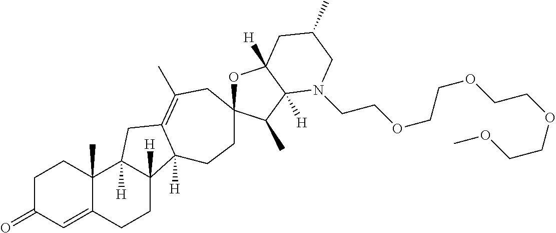

58

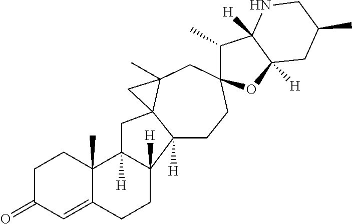

59

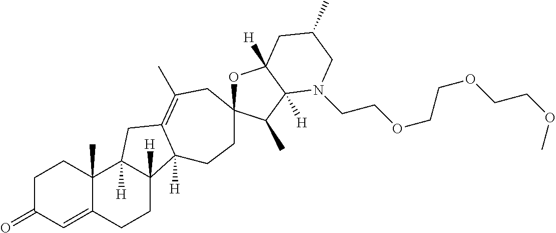

60

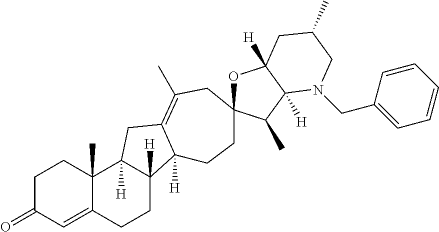

61

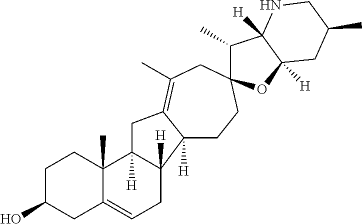

62

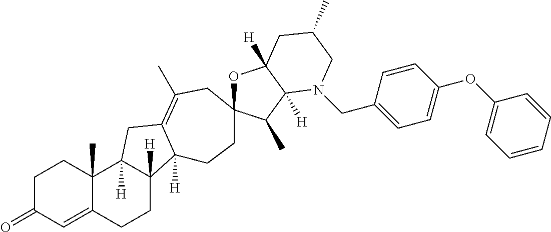

63

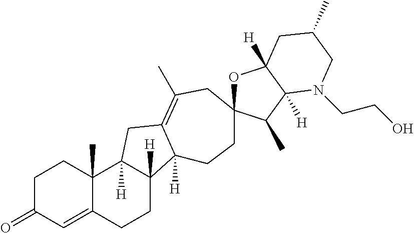

64

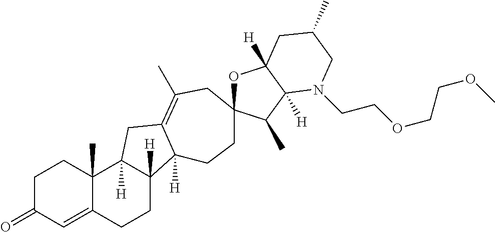

65

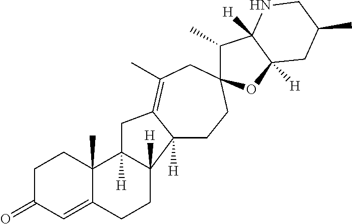

66

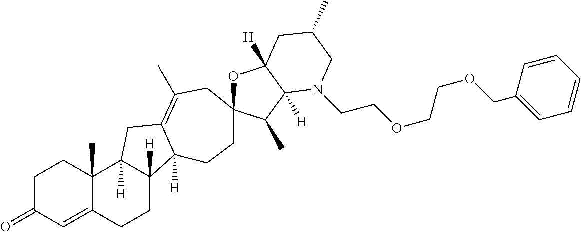

67

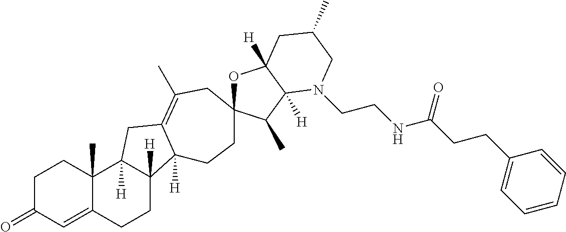

68

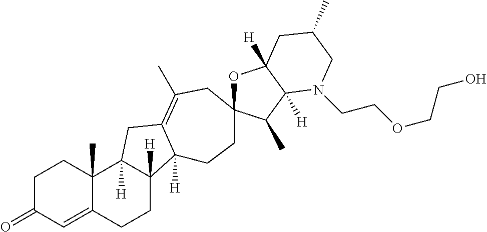

69

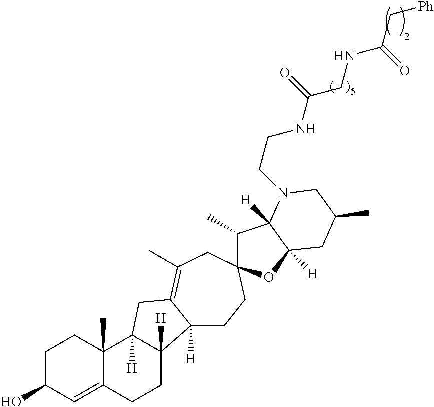

70

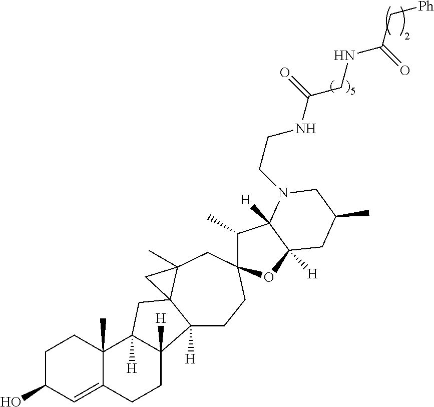

71

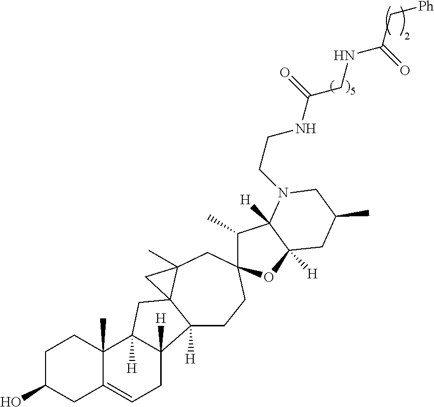

72

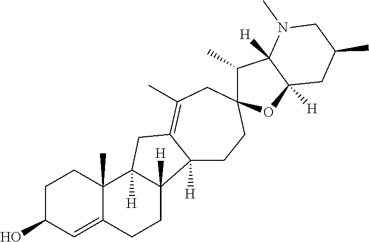

73

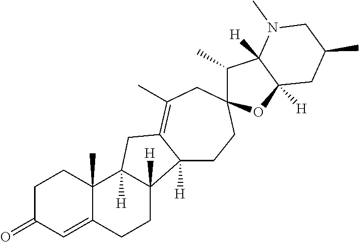

74

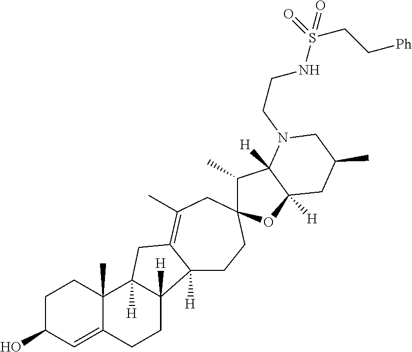

75

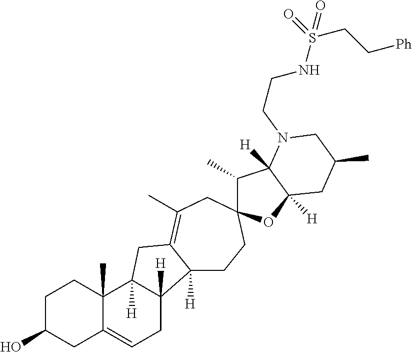

76

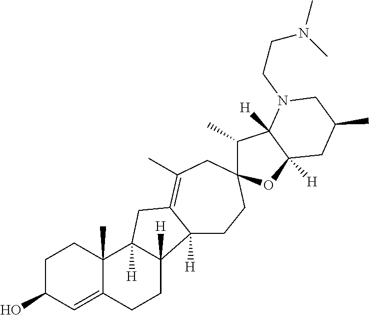

77

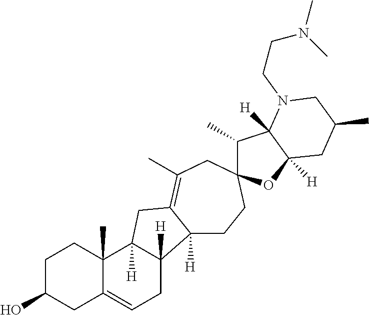

78

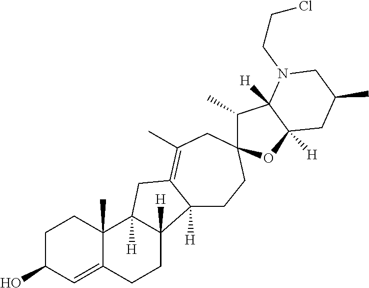

79

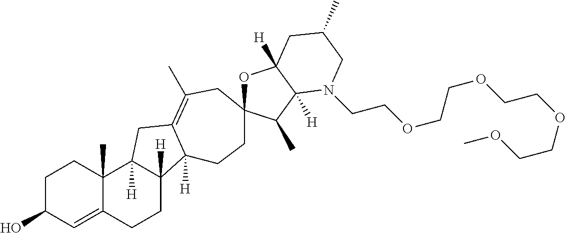

80

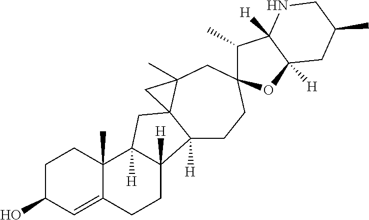

81

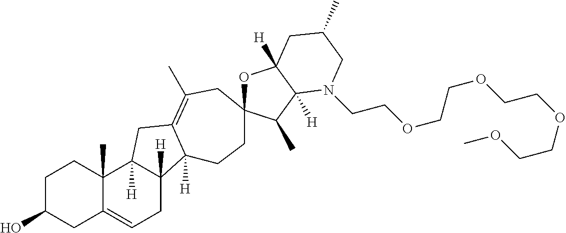

82

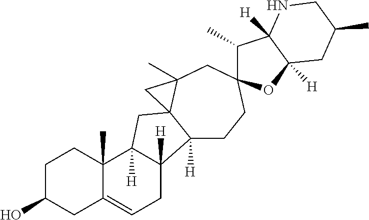

83

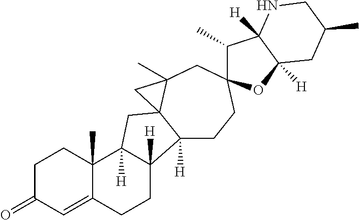

84

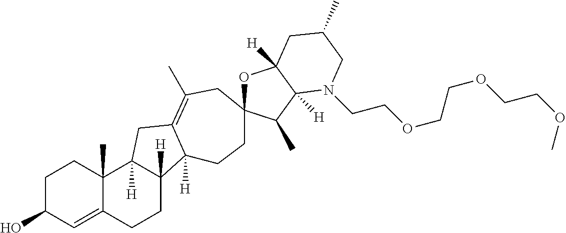

85

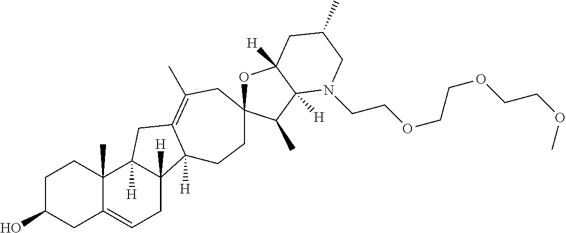

86

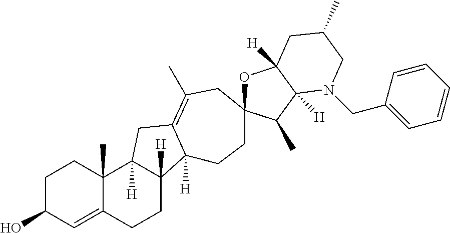

87

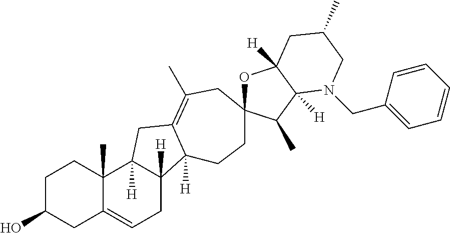

88

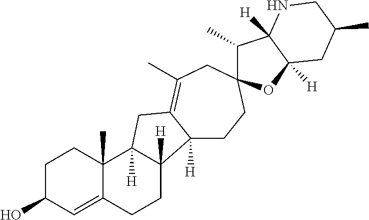

89

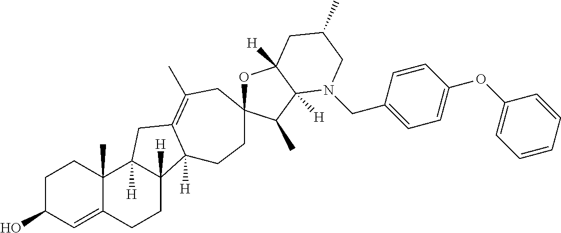

90

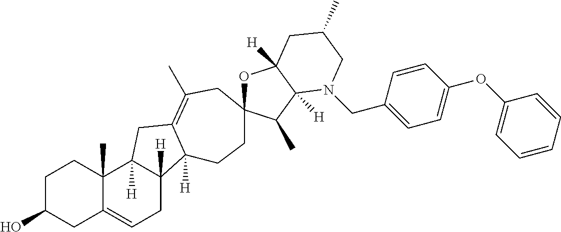

91

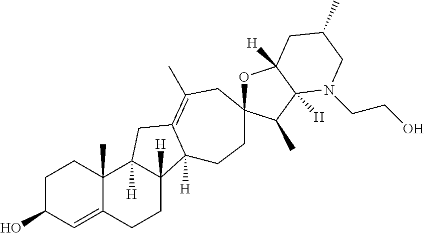

92

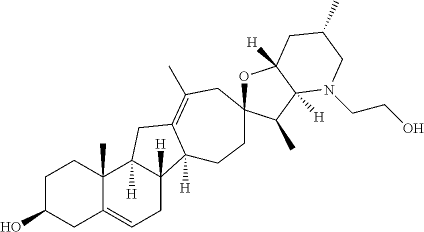

93

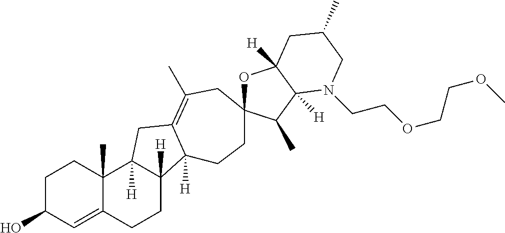

94

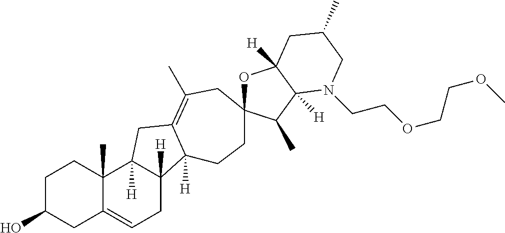

95

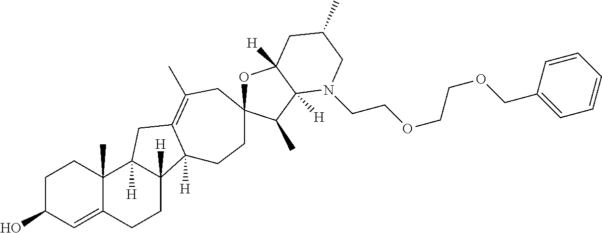

96

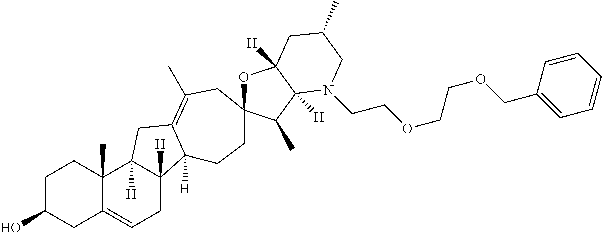

97

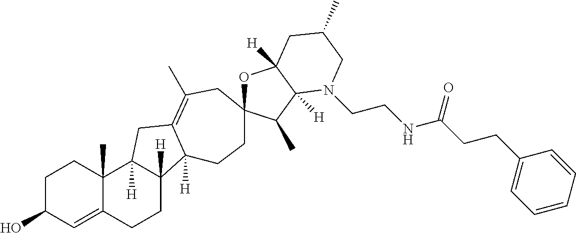

98

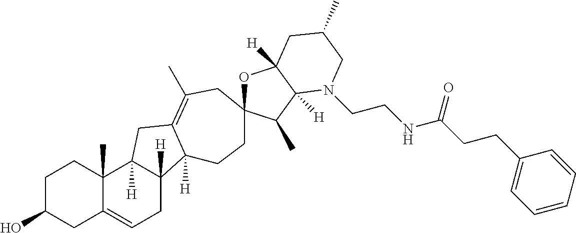

99

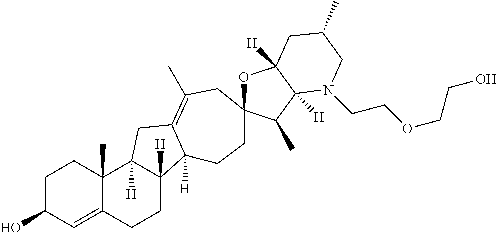

100

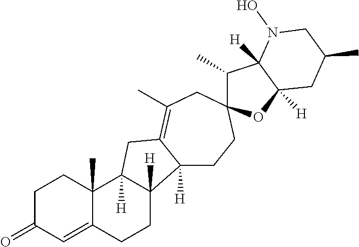

101

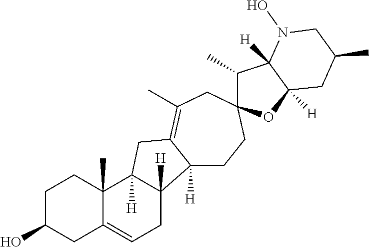

102

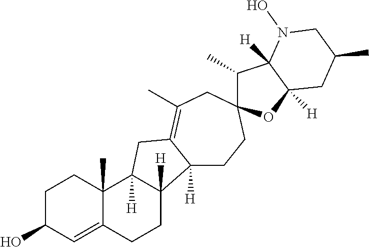

103

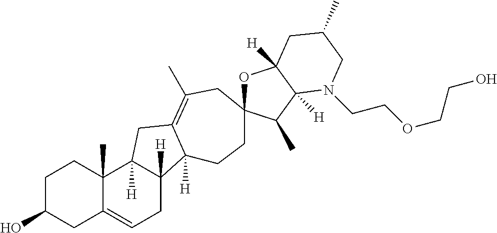

Yet other examples of hedgehog inhibitors include compounds, or pharmaceutically acceptable salts and/or solvates thereof, described in U.S. patent application No. 2008/0287420, and also provided below in Table 3:

TABLE 3
104

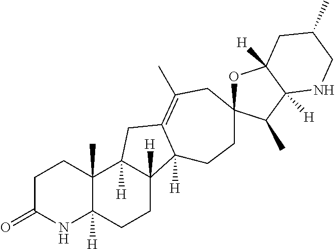

105

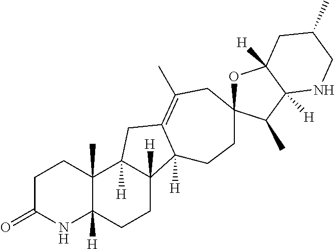

106

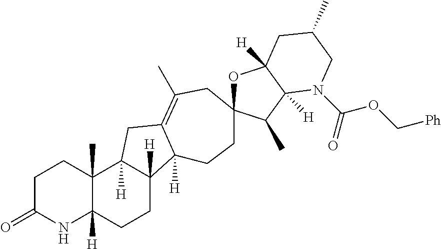

107

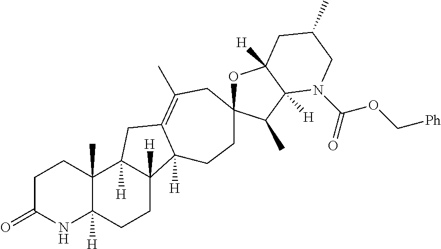

108

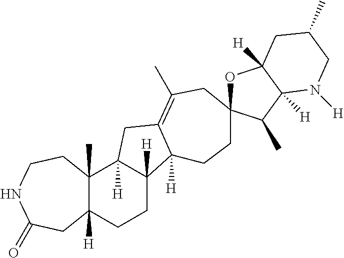

109

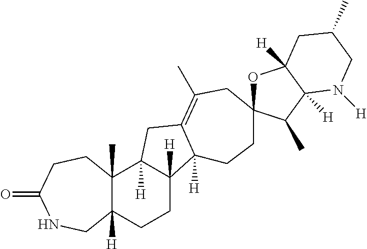

110

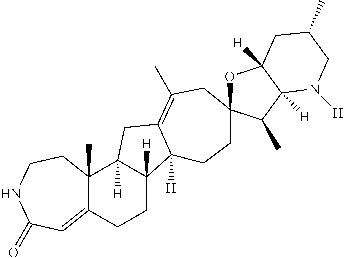

111

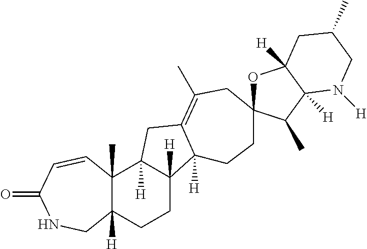

112

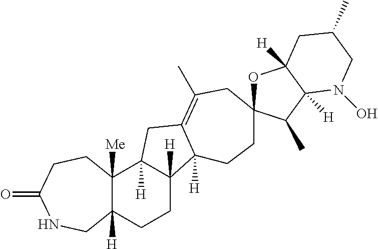

113

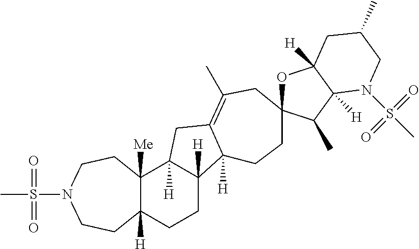

114

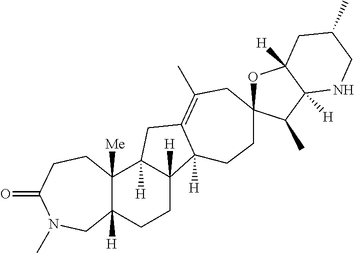

115

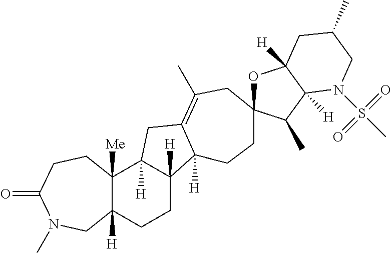

116

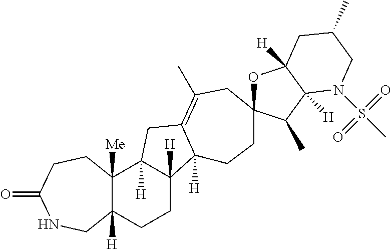

117

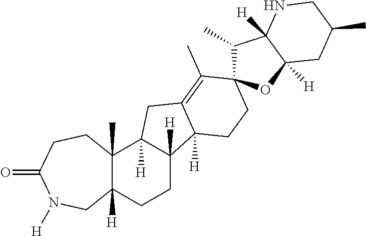

118

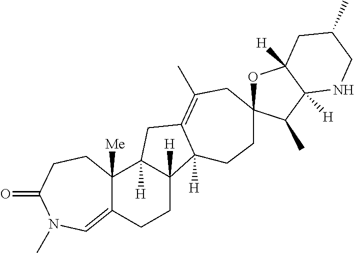

Still yet other examples of hedgehog inhibitors include compounds, or pharmaceutically acceptable salts and/or solvates thereof, described in U.S. patent application No. 2008/0293755, and also provided below in Table 4:

TABLE 4
119

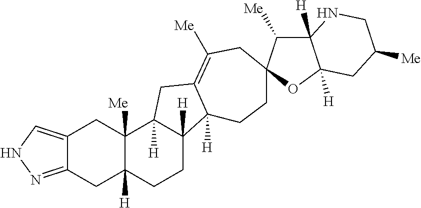

120

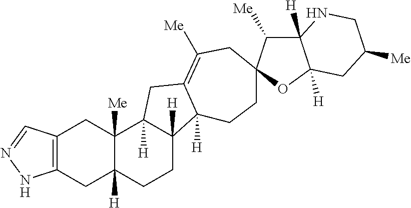

121

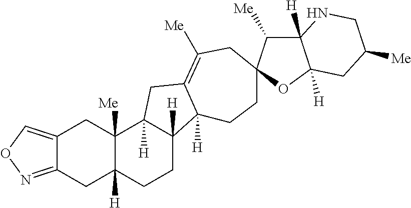

122

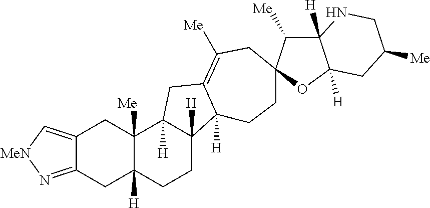

123

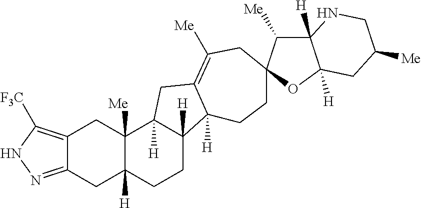

124

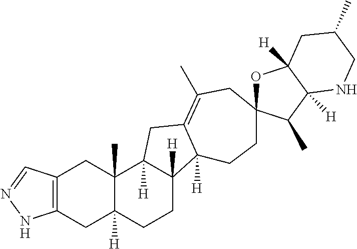

125

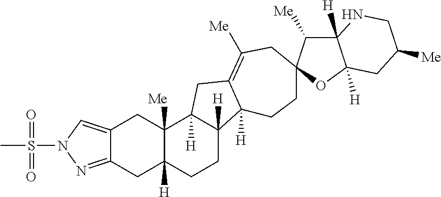

126

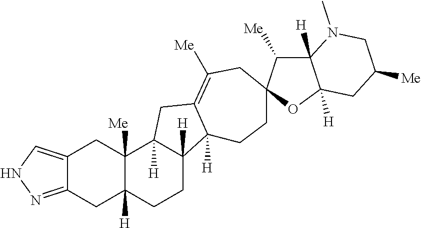

127

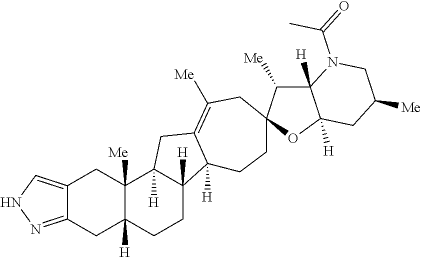

128

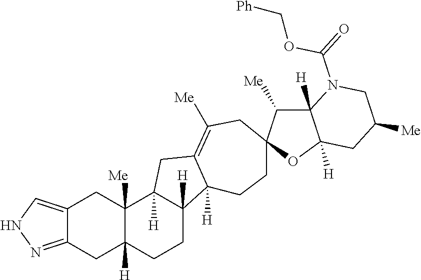

129

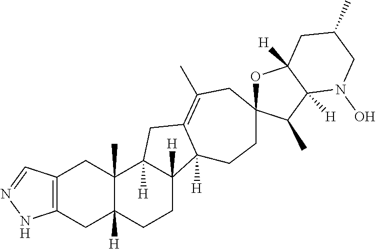

130

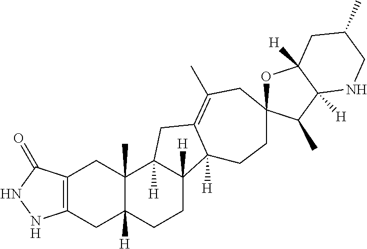

131

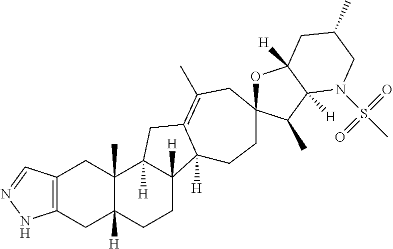

132

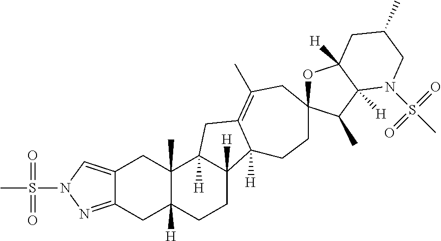

133

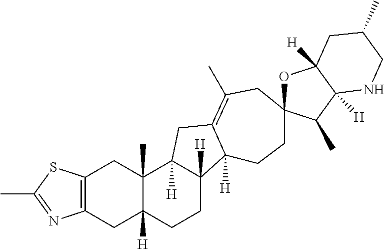

134

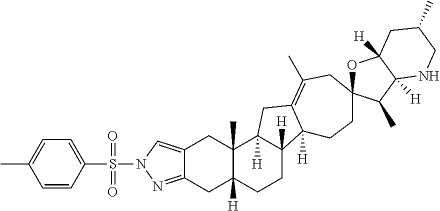

135

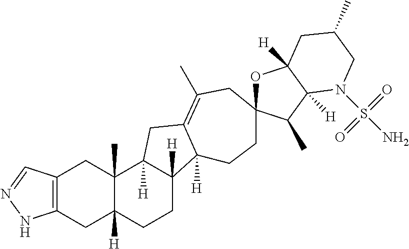

136

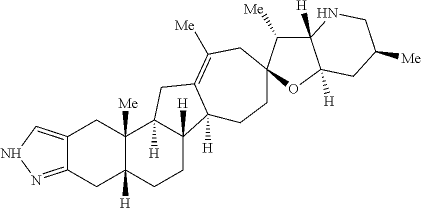

137

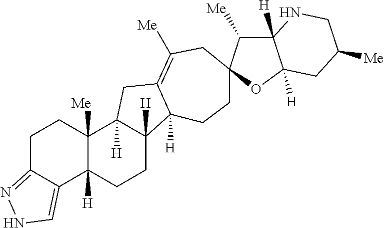

In certain embodiments, the hedgehog inhibitor is the compound 32:

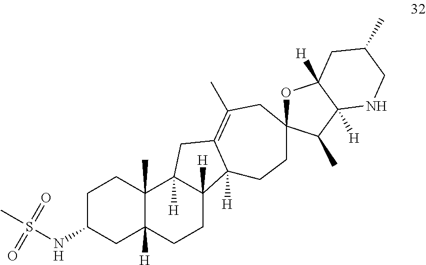

(also referred to herein as IPI-926)

or a pharmaceutically acceptable salt and/or solvate thereof.

Hedgehog inhibitors useful in the current invention can contain a basic functional group, such as amino or alkylamino, and are thus capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately treating the compound in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, besylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, benzenesulfonic, ethane disulfonic, oxalic, isothionic, and the like.

In other cases, the compounds of the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

In certain embodiments, the pharmaceutically acceptable salt of IPI-926 is the hydrochloric, hydrobromic, phosphoric, sulfuric, nitric, perchloric, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic or valeric acid addition salt.

In certain embodiments, the pharmaceutically acceptable salt of IPI-926 is the hydrochloric acid addition salt.

In certain embodiments, the hedgehog inhibitor is an isopropanol (IPA) solvate of IPI-926 or a pharmaceutically acceptable salt thereof.

### Nucleic Acid Molecules

One aspect pertains to nucleic acid molecules that correspond to a marker described herein, including nucleic acids which encode a polypeptide corresponding to a marker of the invention or a portion of such a polypeptide. The nucleic acid molecules of the invention include those nucleic acid molecules which reside in a genomic regions identified herein. Isolated nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

A nucleic acid molecule, e.g., a gene mutations and/or gene products identified herein, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another embodiment, an isolated nucleic acid molecule comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, at least about 15, at least about 25, at least about 50, at least about 75, at least about 100, at least about 125, at least about 150, at least about 175, at least about 200, at least about 250, at least about 300, at least about 350, at least about 400, at least about 500, at least about 600, at least about 700, at least about 800, at least about 900, at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 15 kb, at least about 20 kb, at least about 25 kb, at least about 30 kb, at least about 35 kb, at least about 40 kb, at least about 45 kb, at least about 50 kb, at least about 60 kb, at least about 70 kb, at least about 80 kb, at least about 90 kb, at least about 100 kb, at least about 200 kb, at least about 300 kb, at least about 400 kb, at least about 500 kb, at least about 600 kb, at least about 700 kb, at least about 800 kb, at least about 900 kb, at least about 1 mb, at least about 2 mb, at least about 3 mb, at least about 4 mb, at least about 5 mb, at least about 6 mb, at least about 7 mb, at least about 8 mb, at least about 9 mb, at least about 10 mb or more consecutive nucleotides of a nucleic acid.

Probes based on the sequence of a nucleic acid molecule can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that are substantially homologous to the gene mutations and/or gene products (e.g., the markers set forth herein) such that they are at least 60% to at least 95%, at least 96%, at least 97%, at least 98%, at least 99%, at least 99.5% or greater. In other embodiments, the invention further encompasses nucleic acid molecules that are substantially homologous to the gene mutations and/or gene products (e.g., the markers set forth herein) such that they differ by only or at least 1 to at least 50 kb nucleotides or any range in between.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's can occur in protein-coding nucleic acid sequences, in which case they can give rise to a defective or otherwise variant protein, or genetic disease. Such a SNP can alter the coding sequence of the gene and therefore specify another amino acid (a “missense” SNP) or a SNP can introduce a stop codon (a “nonsense” SNP). When a SNP does not alter the amino acid sequence of a protein, the SNP is called “silent.” SNP's can also occur in noncoding regions of the nucleotide sequence. This can result in defective protein expression, e.g., as a result of alternative spicing, or it can have no effect on the function of the protein.

As used herein, the term “hybridizes under stringent conditions” is intended to describe conditions for hybridization and washing under which nucleotide sequences at least 60%, at least 65%, at least 70%, at least 75%, at least 80%, or at least 85% identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). Another, non-limiting example of stringent hybridization conditions are hybridization in 6× sodium chloride/sodium citrate (SSC) at about 45° C., followed by one or more washes in 0.2×SSC, 0.1% SDS at 50-65° C.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### Proteins and Antibodies

One aspect of the invention pertains to isolated proteins which correspond to individual markers described herein (e.g., a hedgehog marker), and biologically active component or portions thereof. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, less than about 20%, less than about 10%, or less than about 5% (by dry weight) of heterologous protein (also referred to herein as a “contaminating protein”). When the protein or biologically active portion thereof is recombinantly produced, it can be substantially free of culture medium, i.e., culture medium represents less than about 20%, less than about 10%, or less than about 5% of the volume of the protein preparation. When the protein is produced by chemical synthesis, it can substantially be free of chemical precursors or other chemicals, i.e., it is separated from chemical precursors or other chemicals which are involved in the synthesis of the protein. Accordingly such preparations of the protein have less than about 30%, less than about 20%, less than about 10%, less than about 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to gene mutations and/or gene products of the present invention, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

In certain embodiments, the polypeptide has an amino acid sequence of a protein encoded by a nucleic acid molecule described herein. Other useful proteins are substantially identical (e.g., at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 99.5% or greater) to one of these sequences and retain the functional activity of the protein of the corresponding full-length protein yet differ in amino acid sequence.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm (see e.g., Karlin and Altschul (1990) Proc. Natl. Acad. Sci. USA 87:2264-2268, modified as in Karlin and Altschul (1993) Proc. Natl. Acad. Sci. USA 90:5873-5877). Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules.

The percent identity between two sequences can be determined using techniques similar to those described above, with or without allowing gaps. In calculating percent identity, only exact matches are counted.

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (or at least 10, at least 15, at least 20, or at least 30 or more) amino acid residues of the amino acid sequence of one of the polypeptides of the invention, and encompasses an epitope of the protein such that an antibody raised against the peptide forms a specific immune complex with a marker of the invention to which the protein corresponds. Exemplary epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SuriZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to, for example, determine the efficacy of a given treatment regimen. Detection can be facilitated by coupling the antibody to a detectable substance or label. Examples of detectable substances or labels include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

### Kits

A kit is any manufacture (e.g., a package or container) comprising at least one reagent, e.g., a probe, for specifically detecting a marker of the invention, the manufacture being promoted, distributed, or sold as a unit for performing the methods of the present invention. When the compositions, kits, and methods of the invention are used for carrying out the methods of the invention, the markers (e.g., cilium and/or hedgehog markers, gene mutations and/or gene products described herein) can be selected such that a positive result is obtained in at least about 20%, at least about 40%, at least about 60%, at least about 80%, at least about 90%, at least about 95%, at least about 99% or in 100% of subjects afflicted with cancer, of the corresponding stage, grade, histological type, or benign/premaligant/malignant nature. In certain embodiments, the marker or panel of markers of the invention can be selected such that a PPV (positive predictive value) of greater than about 10% is obtained for the general population (e.g., coupled with an assay specificity greater than 99.5%).

When a plurality of cilium and/or hedgehog markers, gene mutations and/or gene products described herein of the invention are used in the compositions, kits, and methods of the invention, the amount, structure, and/or activity of each marker or level of expression or copy number can be compared with the normal amount, structure, and/or activity of each of the plurality of markers or level of expression or copy number, in non-cancerous samples of the same type, either in a single reaction mixture (i.e., using reagents, such as different fluorescent probes, for each marker) or in individual reaction mixtures corresponding to one or more of the cilium and/or hedgehog markers, gene mutations and/or gene products. If a plurality of cilium and/or hedgehog markers, gene mutations and/or gene products described herein is used, then 2, 3, 4, 5, 6, 7, 8, 9, 10, or more individual markers can be used or identified.

The invention includes compositions, kits, and methods for assaying cancer cells in a sample (e.g., an archived tissue sample or a sample obtained from a subject). These compositions, kits, and methods are substantially the same as those described above, except that, where necessary, the compositions, kits, and methods are adapted for use with certain types of samples. For example, when the sample is a parafinized, archived human tissue sample, it can be necessary to adjust the ratio of compounds in the compositions of the invention, in the kits of the invention, or the methods used. Such methods are well known in the art and within the skill of the ordinary artisan.

The invention thus includes a kit for assessing the presence of cancer cells (e.g., in a sample such as a subject sample). The kit can comprise one or more reagents capable of identifying cilium and/or hedgehog markers, gene mutations and/or gene products, e.g., binding specifically with a nucleic acid or polypeptide corresponding to a cilium and/or hedgehog markers, gene mutations and/or gene products described herein. Suitable reagents for binding with a polypeptide corresponding to a marker of the invention include antibodies, antibody derivatives, antibody fragments, and the like. Suitable reagents for binding with a nucleic acid (e.g., a genomic DNA, an mRNA, a spliced mRNA, a cDNA, or the like) include complementary nucleic acids. For example, the nucleic acid reagents can include oligonucleotides (labeled or non-labeled) fixed to a substrate, labeled oligonucleotides not bound with a substrate, pairs of PCR primers, molecular beacon probes, and the like.

The kit of the invention can optionally comprise additional components useful for performing the methods of the invention. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of cancer cells, and the like.

A kit of the invention can comprise a reagent useful for determining protein level or protein activity of a marker. In another embodiment, a kit of the invention can comprise a reagent for determining methylation status of a marker, or can comprise a reagent for determining alteration of structure of a marker, e.g., the presence of a mutation.

### Predictive Medicine

The present invention also pertains to the field of predictive medicine in which diagnostic assays, pharmacogenomics, and monitoring clinical trials are used for predictive purposes to thereby treat an individual prophylactically. Accordingly, one aspect of the present invention relates to assays for determining the amount, structure, and/or activity of polypeptides or nucleic acids corresponding to one or more markers of the invention, in order to determine whether an individual having cancer or at risk of developing cancer will be more likely to respond to hedgehog inhibitor-mediated therapy.

Accordingly, in one aspect, a method for determining whether a subject with a cancer is likely to respond to treatment with a hedgehog inhibiting agent is disclosed. In another aspect, a method for predicting a time course of disease is disclosed. In still another aspect, the method is drawn to a method for predicting a probability of a significant event in the time course of the disease. In certain embodiments, the method comprises detecting a biomarker or combination of biomarkers associated with responsiveness to treatment with a hedgehog inhibiting agent as described herein, and determining whether the subject is likely to respond to treatment with the hedgehog inhibiting agent.

In some embodiments, the methods involve evaluation, e.g., cytogenetic screening, of biological tissue sample from a subject, e.g., a patient who has been diagnosed with or is suspected of having cancer (e.g., presents with symptoms of cancer) to detect one or more hedgehog biomarkers as described herein.

Representative, non-limiting examples of cytogenetic abnormalities that are screened include one or more of the following: EML4-ALK fusions, KIF5B-ALK fusions, TGF-ALK fusions, NPM-ALK fusions, ALK gene copy number changes, and ALK point mutations comprising one or more of F1245I/L, L1204F, A1200V, L1196M, I1170S, T1151M, R1275Q, F1174V/C/L, T1087I, and K1062M.

In other embodiments, the methods involve evaluation, e.g., cytogenetic screening, of biological tissue sample from a subject, e.g., a patient who has been diagnosed with or is suspected of having cancer (e.g., presents with symptoms of cancer) to detect one or more alteration in KRAS, TGFβ-SMADs, p53, cyclin D1, or Gli1. Examples of gene mutations are described in e.g., The Catalogue of Somatic Mutations in Cancer (COSMIC) (http://www.sanger.ac.uk/genetics/CGP/cosmic/).

Additional examples of gene or gene product that can be evaluated can be chosen from ALK, EGFR, PIK3CA, BRAF, PTEN, AKT, TP53, NRAS, CTNNB1 (beta-catenin), APC, KIT, JAK2, NOTCH, or FLT3.

Examples of EGFR mutations are described in e.g., Couzin J., (2004) Science 305:1222-1223; Fukuoka, M. et al., (2003) J. Clin. Oncol. 21:2237-46; Lynch et al., (2004) NEJM 350(21):2129-2139; Paez et al. (2004) Science 304:1497-1500; Pao, W. et al. Proc Natl Acad Sci USA. (2004) 101(36):13306-11; Gazdar A. F. et al., Trends Mol. Med. (2004) 10(10):481-6; Huang S. F. et al. (2004) Clin Cancer Res. 10(24):8195-203; Couzin J. Science (2004) 305(5688):1222-3; Sordella R. et al. (2004) 305(5687):1163-7; Kosaka T. et al. (2004) Cancer Res. 64(24):8919-23; Marchetti A. et al. J Clin Oncol. (2005) 23(4):857-65; Tokumo M. et al. (2005) Clin Cancer Res. 11(3):1167-1173; Han S. W. et al. (2005) J Clin Oncol. 23(11):2493-501; Mitsudomi T. et al. (2005) J Clin Oncol. 23(11):2513-20; Shigematsu H. et al. J Natl Cancer Inst. 97(5):339-46; Kim K. S. et al., (2005) Clin Cancer Res. 11(6):2244-51; Cappuzzo F. et al. (2005) J Natl Cancer Inst. 97(9):643-55; Cortes-Funes H. et al. Ann Oncol. (2005) 16(7):1081-6; Sasaki H. et al. (2005) Clin Cancer Res. 11(8):2924-9; Chou T. Y. et al., (2005) Clin Cancer Res. 11(10):3750-7; Pao W. et al. (2005) PLoS Med. 2(3):e73; Sasaki H. et al. (2005) Int J. Cancer. 118(1):180-4; Eberhard D. A. et al. (2005) J Clin Oncol. 23(25):5900-9; Takano T. et al. (2005) J Clin Oncol. 23(28):6829-37; Tsao M. S. et al., (2005) N Engl J. Med. 353(2):133-44; Mu X. L. et al. (2005) Clin Cancer Res. 11(12):4289-94; Sonobe M. et al. (2005) Br J. Cancer. 93(3):355-63; Taron M. et al. (2005) Clin Cancer Res. 11(16):5878-85; Mukohara T. et al., (2005) J Natl Cancer Inst. 97(16):1185-94; Zhang X. T. et al. (2005) Oncol. 16(8):1334-42.

Examples of KRAS mutations are described in e.g., Tang W. Y. et al. (1999) Br J Cancer 81(2):237-41; Burmer G. C. et al. (1989) Proc. Natl. Acad. Sci. U.S.A. 86(7): 2403-7; Almoguera C. et al. (1988) Cell 53(4): 549-54; Tam I. Y. et al. (2006), Clin. Cancer Res. 12(5): 1647-53.

Examples of PIK3CA mutations are described in e.g., Samuels Y. et al. (2004) Science 304(5670):554; Kurds E. et al. (2004) Cancer Biology & Therapy 3(8):772-775; Stemke-Hale K. et al. (2008) Cancer Res. 68(15):6084-91.

Examples of BRAF mutations are described in e.g., Davies H. et al. (2002) Nature 417: 949-954.

Examples of PTEN mutations are described in e.g., Minaguchi T. et al. (2001) Clin Cancer Res. 7(9):2636-42; Latta E. et al. (2002) Curr Opin Obstet. Gynecol. 14(1):59-65; Eng C. (2003) Hum Mutat. 22(3):183-98; Konopka B. et al. (2002) Cancer Lett. 178(1):43-51; Stemke-Hale K. et al. (2008) Cancer Res. 68(15):6084-91.

Examples of AKT mutations are described in e.g., Stemke-Hale K. et al. (2008) Cancer Res. 68(15):6084-91; Davies M. A. et al. (2008) Br J. Cancer. 99(8):1265-8; Askham J. M. (2010) Oncogene 29(1):150-5; Shoji K. et al (2009) Br J. Cancer. 101(1):145-8.

Examples of TP53 mutations are described in e.g., Soussi T. (2007) Cancer Cell 12(4):303-12; Cheung K. J. (2009) Br J. Haematol. 146(3):257-69; Pfeifer G. P. et al. (2009) Hum Genet. 125(5-6):493-506; Petitjean A. et al. (2007) Oncogene 26(15):2157-65.

Examples of NRAS mutations are described in e.g., Bacher U. et al. (2006) Blood 107:3847-53; Banerji U. et al. (2008) Mol Cancer Ther. 7:737-9.

Examples of CTNNB1 (beta-catenin) mutations are described in e.g., Polakis P. et al. (2000) Genes Dev. 14(15):1837-51; Miyaki M. et al. (1999) Cancer Res. 59(18):4506-9; Tejpar S. et al. (1999) Oncogene 18(47):6615-20; Garcia-Rostan G. et al. (1999) Cancer Res. 59(8):1811-5; Chan E. F. et al. (1999) Nat. Genet. 21(4):410-3; Legoix P. et al. (1999) Oncogene 18(27):4044-6; Mirabelli-Primdahl L. et al. (1999) Cancer Res. 59(14):3346-51.

Examples of NOTCH mutations are described in e.g., Collins B. J. et al. (2004) Semin Cancer Biol. 14(5):357-64; Callahan R. et al. (2001) J Mammary Gland Biol Neoplasia. 6(1):23-36; Mansour M. R. et al. (2006) Leukemia 20:537-539; de Celis J. F. et al. (1993) Proc Natl Acad Sci USA. 90(9):4037-41.

Examples of FLT3 mutations are described in e.g., Kiyoi H. et al. (2006) Methods Mol. Med. 125:189-97; Small D. (2006) Hematology Am Soc Hematol Educ Program. 2006:178-84; Kiyoi H. et al. (2006) Int J. Hematol. 2006 May; 83(4):301-8; Schnittger S. et al. (2004) Acta Haematol. 112(1-2):68-78.

Examples of ERBB2 mutations are described in e.g., U.S. patent application Publication Number 2008/0206248; Lee J. W. et al. (2006) Clin Cancer Res. 12(1):57-61; Lee J. W. et al. (2006) Cancer Lett. 237(1):89-94; Cancer Genome Atlas Research Network (2008) Nature 455(7216):1061-8.

Examples of hedgehogAA1 mutations are described in e.g., Cancer Genome Atlas Research Network (2008) Nature 455(7216):1061-8; Parsons D. W. et al. (2008) Science 321; 1807-12; Sjöblom T. et al. (2006) Science 314; 268-74.

Examples of hedgehogAB1 mutations are described in e.g., Dalgliesh G. L. et al. (2010) Nature 463; 360-3; Parsons D. W. et al. (2008) Science 321; 1807-12; Sjöblom T. et al. (2006) Science 314; 268-74.

Examples NF1 mutations are described in e.g., Thomson S. A. et al. (2002) J Child Neurol. 17(8):555-61; Bottillo I. et al. (2009) J. Pathol. 217(5):693-701; Kluwe L. et al. (2003) J Med. Genet. 40(5):368-71.

Examples of STK11 mutations are described in e.g., Resta N. et al. (1998) Cancer Res. 58(21):4799-801; Nishioka Y. et al. (1999) Jpn J Cancer Res. 90(6):629-32; Marignani P. A. (2005) J Clin Pathol. 58(1):15-9; Katajisto P. et al. (2007) Biochim Biophys Acta. 1775(1):63-75.

Additional examples of gene or gene product that can be evaluated include genes that are differentially expressed in chondrosarcoma. Non-limiting examples of such biomarkers include ADAMTSL1, BOK, C7, CES1, CNR1, DUSP10, FAM150B, FLJ38379, FRMD3, GDF10, Gli1, HGF, HhIP, ITGB3, KCNIP1, LAMA1, LOC339240, MEGF11, PLCXD3, RBP4, SFN, SHANK2, WIF1, FGF18, UBD, ANGPTL7 and SLC2A4. The GenBank™ accession numbers for each of the biomarkers is presented in the following table.

TABLE 1
Exemplary biomarkers differentially expressed in chondrosarcoma

| GenBank ™ Accession No. | Biomarker | Description |
|---|---|---|
| NM_001040272 | ADAMTSL1 | ADAMTS-like 1 |
| NM_032515 | BOK | BCL2-related ovarian killer |
| NM_000587 | C7 | complement component 7 |
| NM_001266 | CES1 | carboxylesterase 1 |
| NM_033181 | CNR1 | cannabinoid receptor 1 |
| | | (brain) |
| NM_007207 | DUSP10 | dual specificity phosphatase |
| | | 10 |
| NM_001002919 | FAM150B | family with sequence |
| | | similarity 150, member B |
| AK095698 | FLJ38379 | hypothetical FLJ38379 |
| BG216229 | FRMD3 | FERM domain containing 3 |
| NM_004962 | GDF10 | growth differentiation factor |
| | | 10 (BMP family) |
| NM_005269 | GLI1 | GLI family zinc finger 1 |
| NM_001010931 | HGF | hepatocyte growth factor |
| | | (hepapoietin A |
| NM_022475 | HhIP | hedgehog interacting |
| | | protein |
| BC007638 | HhIPL2 | HhIP-like 2 |
| NM_000212 | ITGB3 | integrin, beta 3 (platelet |
| | | glycoprotein IIIa, antigen |
| | | CD61) |
| NM_014592 | KCNIP1 | Kv channel interacting |
| | | protein 1 |
| NM_005559 | LAMA1 | laminin, alpha 1 |
| NR_001443 | LOC339240 | keratin pseudogene |
| NM_032445 | MEGF11 | multiple EGF-like-domains |
| | | 11 |
| NM_001005473 | PLCXD3 | phosphatidylinositol- |
| | | specific phospholipase C, X |
| | | domain containing 3 |
| NM_006744 | RBP4 | retinol binding protein 4, |
| | | plasma |
| NM_006142 | SFN | stratifin |
| NM_012309 | SHANK2 | SH3 and multiple ankyrin |
| | | repeat domains 2 |
| NM_177550 | SLC13A5 | solute carrier family 13 |
| | | (sodium-dependent citrate |
| | | transporter), member 5 |
| NM_007191 | WIF1 | WNT inhibitory factor 1 |
| NM_003862 | FGF18 | fibroblast growth factor 18 |
| NM_006398 | UBD | ubiquitin D (FAT10) |
| NM_021146 | ANGPTL7 | angiopoietin-like 7 |
| NM_001042 | SLC2A4 | solute carrier family 2 |
| | | (facilitated glucose |
| | | transporter), member 4 |

Any additional alteration known in the art can be evaluated or treated using the methods of the invention are known in the art.

The results of the screening method and the interpretation thereof are predictive of the patient's response to treatment with hedgehog inhibiting agents. According to the present invention, the presence of one or more cilium markers, and/or alterations in a hedgehog biomarker, e.g., a gene or gene product associated with hedgehog signaling, e.g., a mutation, is indicative that treatment with hedgehog inhibiting agents can provide enhanced therapeutic benefit against the cancer cells relative to those of patients not having the marker.

As discussed further herein, a variety of methods and techniques that are well known in the art can be used for the screening analysis, including metaphase cytogenetic analysis by standard karyotype methods, FISH, spectral karyotyping or MFISH, and comparative genomic hybridization.

In one embodiment, the methods of the present invention comprise contacting a DNA sample, e.g., a genomic DNA sample, such as a chromosomal sample, obtained from cells isolated from the patient to polynucleotide probes that are specific for and hybridize under stringent conditions with genomic DNA in chromosomal regions associated with cytogenetic abnormalities to determine the presence or absence of one or more of the abnormalities in the cells of the patient. The results of the analysis are predictive of the patient's likely response to treatment with therapeutic agents, particularly agents that inhibit hedgehog signaling.

In another embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to metastatic disease. In another embodiment, the significant event is the progression from primary diagnosis to relapse. In another embodiment, the significant event is the progression from metastatic disease to death. In another embodiment, the significant event is the progression from metastatic disease to relapse. In another embodiment, the significant event is the progression from relapse to death. In certain embodiments, the time course is measured with respect to overall survival rate, time to progression and/or using the RECIST or other response criteria.

In certain embodiments, a predetermined measure is created after evaluating the sample by dividing subject's samples into at least two patient subgroups. In certain embodiments, the number of subgroups is two so that the patient sample is divided into a subgroup of patients having one or more of the hedgehog markers described herein, and a subgroup not having the abnormalities. In certain embodiments, the hedgehog marker status in the subject is compared to either the subgroup having or not having the hedgehog marker; if the patient has the hedgehog marker, then the patient is likely to respond to a hedgehog inhibitor (e.g., IPI-926) and/or the patient has an increased likelihood, or is likely, to have a long time course. In certain embodiments, the number of subgroups is greater than two, including, without limitation, three subgroups, four subgroups, five subgroups and six subgroups, depending on stratification of predicted hedgehog inhibitor efficacy as correlated with particular hedgehog abnormalities. In certain embodiments, likeliness to respond is measured with respect to overall survival rate, time to progression and/or using the RECIST criteria. In certain embodiments, the hedgehog inhibitor is IPI-926.

In another aspect, the invention is drawn to a method for determining whether a subject with an alteration in a hedgehog biomarker is likely to respond to treatment with a hedgehog inhibiting agent and/or the time course of disease is long. In another aspect, the invention is drawn to a method for predicting a time course of disease in a subject with a hedgehog marker as described herein. In another aspect, the invention is drawn to a method for predicting the probability of a significant event in a subject with the hedgehog marker.

### Methods for Protein Detection

The cilium and/or hedgehog marker protein can be detected and/or quantified by detecting or quantifying the expressed polypeptide. The polypeptide can be detected and quantified by any of a number of means known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining whether cells express a marker of the present invention.

Another agent for detecting a polypeptide is an antibody capable of binding to a polypeptide corresponding to a marker of the invention, e.g., an antibody with a detectable label. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Immunohistochemistry or IHC refers to the process of localizing antigens (e.g. proteins) in cells of a tissue section exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used in research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as fluorescein, rhodamine, DyLight Fluor or Alexa Fluor.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies can be directly labeled or alternatively can be subsequently detected using labeled antibodies (e.g., labeled sheep anti-human antibodies) that specifically bind to the anti-polypeptide.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In another embodiment, the capture agent is an antibody that specifically binds a polypeptide. The antibody (anti-peptide) can be produced by any of a number of means well known to those of skill in the art.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one embodiment, the labeling agent is a second human antibody bearing a label. Alternatively, the second antibody can lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Exemplary immunoassays for detecting a polypeptide can be competitive or noncompetitive. Noncompetitive immunoassays are assays in which the amount of captured analyte is directly measured. In one “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In another embodiment, the antibody is immobilized on a solid substrate. The amount of polypeptide bound to the antibody can be determined either by measuring the amount of polypeptide present in a polypeptide/antibody complex, or alternatively by measuring the amount of remaining uncomplexed polypeptide. The amount of polypeptide can be detected by providing a labeled polypeptide.

The assays described herein are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described herein.

In another embodiment, level (activity) is assayed by measuring the enzymatic activity of the gene product. Methods of assaying the activity of an enzyme are well known to those of skill in the art.

In vivo techniques for detection of a marker protein include introducing into a subject a labeled antibody directed against the protein. For example, the antibody can be labeled with a radioactive marker whose presence and location in a subject can be detected by standard imaging techniques.

Certain markers identified by the methods of the invention can be secreted proteins. It is a simple matter for the skilled artisan to determine whether any particular marker protein is a secreted protein. In order to make this determination, the marker protein is expressed in, for example, a mammalian cell, e.g., a human cell line, extracellular fluid is collected, and the presence or absence of the protein in the extracellular fluid is assessed (e.g., using a labeled antibody which binds specifically with the protein).

It will be appreciated that subject samples, e.g., a sample containing tissue, whole blood, serum, plasma, buccal scrape, saliva, cerebrospinal fluid, urine, stool, and bone marrow, can contain cells therein, particularly when the cells are cancerous, and, more particularly, when the cancer is metastasizing, and thus can be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the marker in the sample. Thus, the compositions, kits, and methods of the invention can be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods can be used to detect such proteins on whole cells, or well known computer-based sequence analysis methods (e.g., the SIGNALP program; Nielsen et al., 1997, Protein Engineering 10:1-6) can be used to predict the presence of at least one extracellular domain (i.e., including both secreted proteins and proteins having at least one cell-surface domain). Expression of a marker corresponding to a protein having at least one portion which is displayed on the surface of a cell which expresses it can be detected without necessarily lysing the cell (e.g., using a labeled antibody which binds specifically with a cell-surface domain of the protein).

The invention also encompasses kits for detecting the presence of a polypeptide or nucleic acid corresponding to a marker of the invention in a biological sample, e.g., a sample containing tissue, whole blood, serum, plasma, buccal scrape, saliva, cerebrospinal fluid, urine, stool, and bone marrow. Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

### Methods for Detection of Gene Mutations

Methods of evaluating gene, mutations and/or gene products are well known to those of skill in the art, including hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the presence/absence and relative copy number of the target nucleic acid. Alternatively, a Northern blot can be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the presence/absence and relative copy number of the target nucleic acid.

An alternative means for determining the copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

Exemplary hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g., membrane or glass) bound methods or array-based approaches.

In one aspect, FISH analysis is used. Cell samples are obtained from patients according to methods well known in the art in order to be tested by an appropriate cytogenetic testing method known in the art, for example, the FISH method. In one embodiment, FISH can be performed according to the Vysis™ system (Abbott Molecular), whose manufacturer's protocols are incorporated herein by reference.

Probes are used that contain DNA segments that are essentially complementary to DNA base sequences existing in different portions of chromosomes. Examples of probes useful according to the invention, and labeling and hybridization of probes to samples are described in two U.S. patents to Vysis, Inc. U.S. Pat. Nos. 5,491,224 and 6,277,569 to Bittner, et al.

Chromosomal probes are typically about 50 to about 10⁵ nucleotides in length. Longer probes typically comprise smaller fragments of about 100 to about 500 nucleotides in length. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, Biotechnic Histochem., 1998, 73(1):6-22, Wheeless et al., Cytometry 1994, 17:319-326, and U.S. Pat. No. 5,491,224.

The probes to be used hybridize to a specific region of a chromosome to determine whether a cytogenetic abnormality is present in this region. One type of cytogenetic abnormality is a deletion. Although deletions can be of one or more entire chromosomes, deletions normally involve loss of part of one or more chromosomes. If the entire region of a chromosome that is contained in a probe is deleted from a cell, hybridization of that probe to the DNA from the cell will normally not occur and no signal will be present on that chromosome. If the region of a chromosome that is partially contained within a probe is deleted from a cell, hybridization of that probe to the DNA from the cell can still occur, but less of a signal can be present. For example, the loss of a signal is compared to probe hybridization to DNA from control cells that do not contain the genetic abnormalities which the probes are intended to detect. In some embodiments, at least 1, 5, 10, 20, 30, 40, 50, 60, 70, 80, 90, 100, 110, 120, 130, 140, 150, 160, 170, 180, 190, 200, or more cells are enumerated for presence of the cytogenetic abnormality.

Cytogenetic abnormalities to be detected can include, but are not limited to, non-reciprocal translocations, intra-chromosomal inversions, point mutations, deletions, gene copy number changes, gene expression level changes, and germ line mutations. In particular, one type of cytogenetic abnormality is a duplication. Duplications can be of entire chromosomes, or of regions smaller than an entire chromosome. If the region of a chromosome that is contained in a probe is duplicated in a cell, hybridization of that probe to the DNA from the cell will normally produce at least one additional signal as compared to the number of signals present in control cells with no abnormality of the chromosomal region contained in the probe.

Chromosomal probes are labeled so that the chromosomal region to which they hybridize can be detected. Probes typically are directly labeled with a fluorophore, an organic molecule that fluoresces after absorbing light of lower wavelength/higher energy. The fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, U.S. Pat. No. 5,491,224.

U.S. Pat. No. 5,491,224 describes probe labeling as a number of the cytosine residues having a fluorescent label covalently bonded thereto. The number of fluorescently labeled cytosine bases is sufficient to generate a detectable fluorescent signal while the individual so labeled DNA segments essentially retain their specific complementary binding (hybridizing) properties with respect to the chromosome or chromosome region to be detected. Such probes are made by taking the unlabeled DNA probe segment, transaminating with a linking group a number of deoxycytidine nucleotides in the segment, covalently bonding a fluorescent label to at least a portion of the transaminated deoxycytidine bases.

Probes can also be labeled by nick translation, random primer labeling or PCR labeling. Labeling is done using either fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP or Dinitrophenyl (DNP)-11-dUTP.

Probes also can be indirectly labeled with biotin or digoxygenin, or labeled with radioactive isotopes such as ³²p and .³H, although secondary detection molecules or further processing then is required to visualize the probes. For example, a probe labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Probes can also be prepared such that a fluorescent or other label is not part of the DNA before or during the hybridization, and is added after hybridization to detect the probe hybridized to a chromosome. For example, probes can be used that have antigenic molecules incorporated into the DNA. After hybridization, these antigenic molecules are detected using specific antibodies reactive with the antigenic molecules. Such antibodies can themselves incorporate a fluorochrome, or can be detected using a second antibody with a bound fluorochrome.

However treated or modified, the probe DNA is commonly purified in order to remove unreacted, residual products (e.g., fluorochrome molecules not incorporated into the DNA) before use in hybridization.

Prior to hybridization, chromosomal probes are denatured according to methods well known in the art. In general, hybridization steps comprise adding an excess of blocking DNA to the labeled probe composition, contacting the blocked probe composition under hybridizing conditions with the chromosome region to be detected, e.g., on a slide where the DNA has been denatured, washing away unhybridized probe, and detecting the binding of the probe composition to the chromosome or chromosomal region.

Probes are hybridized or annealed to the chromosomal DNA under hybridizing conditions. “Hybridizing conditions” are conditions that facilitate annealing between a probe and target chromosomal DNA. Since annealing of different probes will vary depending on probe length, base concentration and the like, annealing is facilitated by varying probe concentration, hybridization temperature, salt concentration and other factors well known in the art.

Hybridization conditions are facilitated by varying the concentrations, base compositions, complexities, and lengths of the probes, as well as salt concentrations, temperatures, and length of incubation. For example, in situ hybridizations are typically performed in hybridization buffer containing 1-2×SSC, 50-65% formamide and blocking DNA to suppress non-specific hybridization. In general, hybridization conditions, as described above, include temperatures of about 25° C. to about 55° C., and incubation lengths of about 0.5 hours to about 96 hours.

Non-specific binding of chromosomal probes to DNA outside of the target region can be removed by a series of washes. Temperature and concentration of salt in each wash are varied to control stringency of the washes. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes. In some applications it is necessary to block the hybridization capacity of repetitive sequences. Thus, in some embodiments, tRNA, human genomic DNA, or Cot-I DNA is used to block non-specific hybridization.

After washing, the slide is allowed to drain and air dry, then mounting medium, a counterstain such as DAPI, and a coverslip are applied to the slide. Slides can be viewed immediately or stored at −20° C. before examination.

For fluorescent probes used in fluorescence in situ hybridization (FISH) techniques, fluorescence can be viewed with a fluorescence microscope equipped with an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the chromosomal probes. FISH can be used to detect chromosome copy number or rearrangement of regions of chromosomes. These probes hybridize, or bind, to the complementary DNA and, because they are labeled with fluorescent tags, allow researchers to see the location of those sequences of DNA using a fluorescence microscope. Unlike most other techniques used to study chromosomes, which require that the cells be actively dividing, FISH can also be performed on non-dividing cells, making it a highly versatile procedure. Therefore, FISH can be performed using interphase cells, or cells in metaphase of the cell division cycle. Many of the techniques involved in FISH analysis are described in U.S. Pat. No. 5,447,841 by Gray and Pinkel.

FISH results can be interpreted with reference to control cells that are known not to contain the specific cytogenetic abnormality the probe is designed to detect. The FISH hybridization pattern of the probe to DNA from the control cells is compared to hybridization of the same probe to the DNA from cells that are being tested or assayed for the specific cytogenetic abnormality. When a probe is designed to detect a deletion of a chromosome or chromosomal region, there normally is less hybridization of the probe to DNA from the cells being tested than from the control cells. Normally, there is absence of a probe signal in the tested cells, indicative of loss of the region of a chromosome the probe normally hybridizes to. When a probe is designed to detect a chromosomal duplication or addition, there normally is more hybridization of the probe to DNA from the cells being tested than from the control cells. Normally, there is addition of a probe signal in the tested cells, indicative of the presence of an additional chromosomal region that the probe normally hybridizes to.

In CGH methods, a first collection of nucleic acids (e.g., from a sample, e.g., a possible tumor) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH can also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl. Acad Sci USA 89:5321-5325 (1992) is used. Array-based CGH is described in U.S. Pat. No. 6,455,258, the contents of each of which are incorporated herein by reference.

In still another embodiment, amplification-based assays can be used to measure presence/absence and copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g., healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR can also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) can also be used to identify regions of amplification or deletion.

### Methods for Detection of Gene Expression

Marker expression level can also be assayed. Expression of a marker of the invention can be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that can contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

In another embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An PET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Exemplary nucleic acid probes are 20 bases or longer in length (See, e.g., Sambrook et al. for methods of selecting nucleic acid probe sequences for use in nucleic acid hybridization). Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a marker, the level of expression of the marker is determined for 10 or more samples of normal versus cancer cell isolates, or even 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In certain embodiments, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

In another embodiment, expression of a marker is assessed by preparing genomic DNA or mRNA/cDNA (i.e., a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g., at least 7, at least 10, at least 15, at least 20, at least 25, at least 30, at least 40, at least 50, at least 100, at least 500, or more nucleotide residues) of a marker of the invention. If polynucleotides complementary to or homologous with a marker of the invention are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, the hybridization can be performed under stringent hybridization conditions.

In another embodiment, a combination of methods to assess the expression of a marker is utilized.

Because the compositions, kits, and methods of the invention rely on detection of a difference in expression levels or copy number of one or more markers of the invention, in certain embodiments the level of expression or copy number of the marker is significantly greater than the minimum detection limit of the method used to assess expression or copy number in at least one of normal cells and cancerous cells.

### Method for Detecting Structural Alterations

The invention also provides a method for assessing the presence of a structural alteration, e.g., a mutation.

One detection method is allele specific hybridization using probes overlapping the polymorphic site and having about 5, about 10, about 20, about 25, or about 30 nucleotides around the polymorphic region. In another embodiment of the invention, several probes capable of hybridizing specifically to mutations are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the mutations of at least one polymorphic region of a gene. The solid phase support is then contacted with a test nucleic acid and hybridization to the specific probes is detected. Accordingly, the identity of numerous mutations of one or more genes can be identified in a simple hybridization experiment. For example, the identity of the mutation of the nucleotide polymorphism in the 5′ upstream regulatory element can be determined in a single hybridization experiment.

In other detection methods, it is necessary to first amplify at least a portion of a marker prior to identifying the mutation. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In certain embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a marker and detect mutations by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled DNA Sequencing by Mass Spectrometry Via Exonuclease Degradation by H. Köster), and U.S. Pat. No. 5,605,798 and International patent application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

Other sequencing methods include, but not limited to, in vitro clonal amplification (e.g., as described in Margulies M. et al. (2005) Nature 437 (7057):376-380; Shendure J. (2005) Science 309:1728 (also known as Polony sequencing); SOLid™ sequencing (Applied Biosystem http://www.appliedbiosystems.com/absite/us/en/home/applications-technologies/solid-next-generation-sequencing.html); bridge amplification (Illumina http://www.illumina.com/technology/sequencing_technology.html); Braslaysky I. et al. (2003) Proc. Natl. Acad. Sci. U.S.A. 100(7):3960-3964), parallelized sequencing (e.g., as described in Margulies M. et al. (2005) Nature 437 (7057):376-380; Ronaghi M. et al. (1996) Analytical Biochemistry 242(1):84-89; reversible terminator methods (e.g., used by Illumina and Helicos); pyrosequencing (e.g., used by 454 Life Sciences), sequencing by ligation (e.g., as described in Shendure J. (2005) Science 309:1728; SOLid™ sequencing (Applied Biosystem http://www.appliedbiosystems.com/absite/us/en/home/applications-technologies/solid-next-generation-sequencing.html); U.S. Pat. No. 5,750,341 entitled “DNA sequencing by parallel oligonucleotide extentions”), microfluidic Sanger sequencing, sequencing by hybridization (e.g., non-enzymatic method that uses a DNA microarray as described in Hanna G. J. et al. (2000) J. Clin. Microbiol. 38(7):2715-2721); microscopy-based techniques (e.g., as described in U.S. patent application Publication Number 2006/0029957).

In some cases, the presence of a specific allele of a marker in DNA from a subject can be shown by restriction enzyme analysis. For example, a specific nucleotide polymorphism can result in a nucleotide sequence comprising a restriction site which is absent from the nucleotide sequence of another mutation.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a marker mutation with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl. Acad Sci USA 85:4397; Saleeba et al (1992) Methods Enzymol. 217:286-295. In another embodiment, the control or sample nucleic acid is labeled for detection.

In another embodiment, an mutation can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility are used to identify the type of marker mutation. For example, single strand conformation polymorphism (SSCP) can be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of a mutation of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes can be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci. USA 86:6230; and Wallace et al. (1979) Nucl. Acids Res. 6:3543). Such allele specific oligonucleotide hybridization techniques can be used for the simultaneous detection of several nucleotide changes in different polymorphic regions of marker. For example, oligonucleotides having nucleotide sequences of specific mutations are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it can be desirable to introduce a novel restriction site in the region of the mutation to create cleavage-based detection (Gasparini et al (1992) Mol. Cell. Probes 6:1).

In another embodiment, identification of the mutation is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

The invention further provides methods for detecting single nucleotide polymorphisms in a marker. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087), the method of Goelet, P. et al. is a heterogeneous phase assay, in which the primer or the target molecule is immobilized to a solid phase.

Several primer-guided nucleotide incorporation procedures for assaying polymorphic sites in DNA have been described (Komher, J. S. et al., (1989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mutat. 1:159-164; Ugozzoli, L. et al., (1992) GATA 9:107-112; Nyren, P. (1993) et al., Anal. Biochem. 208:171-175). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the mutation of a polymorphic region located in the coding region of a marker, yet other methods than those described above can be used. For example, identification of a mutation which encodes a mutated marker can be performed by using an antibody specifically recognizing the mutant protein in, e.g., immunohistochemistry or immunoprecipitation. Antibodies to wild-type markers or mutated forms of markers can be prepared according to methods known in the art.

Alternatively, one can also measure an activity of a marker, such as binding to a marker ligand. Binding assays are known in the art and involve, e.g., obtaining cells from a subject, and performing binding experiments with a labeled ligand, to determine whether binding to the mutated form of the protein differs from binding to the wild-type of the protein.

### Hedgehog-Inhibiting Therapeutic Agents, Compositions and Administration

Methods for treating one or more cancers by administering a hedgehog inhibitor are also disclosed. The hedgehog inhibitor is administered in combination with another cancer therapy, such as one or more therapeutic agents, radiation therapy and/or surgery. In one embodiment, the cancer therapy and hedgehog inhibitor can be administered concurrently, sequentially, or a combination of concurrent administration followed by monotherapy with the hedgehog inhibitor.

Expression of Sonic Hedgehog (SHh) ligand is found in tumors of various organs, e.g., pancreas, colon, ovarian and prostate, among others. Administration of a hedgehog inhibitor reduced expression of stromal-derived tumor markers surrounding various cancers, while no significant reduction of tumor activity was detected, thus supporting a paracrine signaling mechanism between the hedgehog-secreting tumors and hedgehog signaling pathway in the surrounding stroma. Hedgehog inhibitors reduced the activity of a hedgehog receptor, e.g., Smoothened and/or Patched, in a tumor microenvironment, thereby causing one or more of: (i) depleting or reducing desmoplastic stroma; (ii) increasing the vascularity of the tumor; or (iii) rendering the tumor more accessible to chemotherapy. Thus, methods and compositions for treating or preventing a cancer by administering to a subject a hedgehog inhibitor, alone or combination with a second therapeutic agent are disclosed. The therapeutic methods can be used in combination with the diagnostic, prognostic methods described herein.

IPI-926, described in PCT publications WO 2008083252 and WO 2008083248, both of which are incorporated herein by reference, has been shown to inhibit in vitro growth of human cell lines derived from patients with pancreatic cancer, medulloblastoma, lung cancer, multiple myeloma, acute lymphocytic leukemia, myelodysplatic syndrome, non-Hodgkin's type lymphoma, Hodgkin's disease and lymphocytic leukemia.

IPI-926 has also shown tumor growth inhibition in a number of preclinical in vivo models, such as medulloblastoma (Pink et al., “Activity of IPI-926, a potent HH pathway inhibitor, in a novel model of medulloblastoma derived from Ptch/HIC +/− mice” American Association for Cancer Research, 1588, 2008; Villavicencia et al., “Activity of the Hh pathway inhibitor IPI-926 in a mouse model of medulloblastoma” American Association for Cancer Research, 2009); small cell lung cancer (Travaglione et al., “A novel Hh pathway inhibitor, IPI-926, delays recurrence post-chemotherapy in a primary human SCLC xenograft model,” American Association for Cancer Research, 4611, 2008; Peacock et al., “Visualization of smoothened activation supports an essential role for Hedgehog signaling in the regulation of self-renewal in small cell lung cancer” American Association for Cancer Research, 2009); non-small cell lung cancer (Mandley, E., et al. The Hh inhibitor IPI-926 delays tumor re-growth of a non-small cell lung cancer xenograft model following treatment with an EGFR targeted tyrosine kinase inhibitor. American Association for Cancer Research, 2010), skin cancer, head and neck cancer, and ovarian cancer (Growdon et al, “Hedgehog pathway inhibitor cyclopamine suppresses Gli1 expression and inhibits serous ovarian cancer xenograft growth.” Society of Gynecologic Oncologists Annual Meeting on Women's Cancer, 2009).

Additionally, hedgehog inhibitors, e.g., IPI-926, have demonstrated rapid and sustained Hedgehog pathway inhibition in stromal cells, a downstream mediator of Hedgehog signaling, after single administration in a model of human pancreatic cancer (Traviglione et al., EORTC-NCI-AACR Symposium on “Molecular Targets and Cancer Therapeutics” 2008).

Inhibition of the hedgehog pathway has also been shown to reduce or inhibit the growth of a variety of cancers, such as acute lymphocytic leukemia (ALL) (Ji et al., Journal of Biological Chemistry (2007) 282:37370-37377); basal cell carcinoma (Xie et al., Nature (1998) 391:90-92; Williams et al., PNAS (2003) 100:4616-4621; Bale and Yu (2001) Human Molecular Genetics (2001) 10:757-762); biliary cancer (Berman et al., Nature (2003) 425:846-851; WO 2005/013800); brain cancer and glioma (Clement et al., Current Biology (2007) 17:1-8; Ehtesham et al., Oncogene (2007) 1-10); bladder cancer; breast cancer (Kubo et al., Cancer Research (2004) 64:6071-6074; Lewis et al., J. Mammary Gland Biology and Neoplasia (2004) 2:165-181); chondrosarcoma (Wunder et al., Lancet Oncology (2007) 513-524); chronic lymphocytic leukemia (CLL) (Hedge et al., Mol. Cancer. Res.(2008) 6:1928-1936); chronic myeloid leukemia (CML) (Dierks et al., Cancer Cell (2008) 14:238-249); colon cancer (Yang and Hinds, BMC Developmental Biology (2007) 7:6); esophageal cancer (Berman et al., Nature (2003) 425:846-851; WO 2005/013800); gastric cancer (Berman et al., Nature (2003) 425:846-851; Ma et al., Carcinogenesis (2005) 26:1698-1705; WO 2005/013800; Shiotani et al., J. Gastroenterol. Hepatol. (2008) S161-S166; Ohta et al., Cancer Research (2005) 65:10822-10829; Ma et al., World J. Gastroenterol (2006) 12:3965-3969); gastrointestinal stromal tumor (GIST) (Yoshizaki et al., World J. Gastroenterol (2006) 12:5687-5691); hepatocellular cancer (Sicklick et al., Carcinogenesis (2006) 27:748-757; Patil et al., Cancer Biology & Therapy (2006) 5:111-117); kidney cancer (Cutcliffe et al., Human Cancer Biology (2005) 11:7986-7994); lung cancer (Watkins et al., Nature (2003) 422:313-317); medulloblastoma (Berman et al., Science (2002) 297:1559-1561; Pietsch et al. Cancer Research (1997) 57:2085-2088); melanoma (Stecca et al., PNAS (2007) 104:5895-5900; Geng et al., Angiogenesis (2007) 10:259-267); multiple myeloma (Peacock et al., PNAS USA (2007) 104:4048-4053; Dierks et al., Nature Medicine (2007) 13:944-951); neuroectodermal tumors (Reifenberger et al., Cancer Research (1998) 58:1798-1803); non-Hodgkin's type lymphoma (NHL) (Dierks et al., Nature Medicine (2007) 13:944-951; Lindemann, Cancer Research (2008) 68:961-964); osteosarcoma (Warzecha et al., J. Chemother. (2007) 19:554-561); ovarian cancer (Steg et al., J. Molecular Diagnostics (2006) 8:76-83); pancreatic cancer (Thayer et al., Nature (2003) 425:851-856; Berman et al., Nature (2003) 425:846-851; WO 2005/013800); prostate cancer (Karhadkar et al., Nature (2004) 431:707-712; Sheng et al., Molecular Cancer (2004) 3:29-42; Fan et al., Endocrinology (2004) 145:3961-3970); and testicular cancer (Dormeyer et al., J. Proteome Res. (2008) 7:2936-2951).

In one aspect, the invention relates to a method of treating cancer by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a single therapeutic agent, or multiple therapeutic agents administered sequentially or in combination.

In another aspect, the invention relates to a method of treating cancer including the steps of administering to a patient a first therapeutic agent, then administering the first therapeutic agent in combination with a second therapeutic agent, wherein the second therapeutic agent is a hedgehog inhibitor.

In another aspect, the invention relates to a method of treating a condition mediated by the hedgehog pathway by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a therapeutic agent. In another aspect, the invention relates to a method of treating a condition mediated by the hedgehog pathway including the steps of administering to a patient a first therapeutic agent, then administering the first therapeutic agent in combination with a second therapeutic agent, wherein the second therapeutic agent is a hedgehog inhibitor.

The invention also relates to methods of extending relapse free survival in a cancer patient who is undergoing or has undergone cancer therapy (for example, treatment with one or more therapeutic agents, radiation and/or surgery) by administering a therapeutically effective amount of a hedgehog inhibitor to the patient. “Relapse free survival”, as understood by those skilled in the art, is the length of time following a specific point of cancer treatment during which there is no clinically-defined relapse in the cancer. In some embodiments, the hedgehog inhibitor is administered concurrently with the cancer therapy. In instances of concurrent administration, the hedgehog inhibitor can continue to be administered after the cancer therapy has ceased. In other embodiments, the hedgehog inhibitor is administered after cancer therapy has ceased (i.e., with no period of overlap with the cancer treatment). The hedgehog inhibitor can be administered immediately after cancer therapy has ceased, or there can be a gap in time (e.g., up to about a day, a week, a month, six months, or a year) between the end of cancer therapy and the administration of the hedgehog inhibitor. Treatment with the hedgehog inhibitor can continue for as long as relapse-free survival is maintained (e.g., up to about a day, a week, a month, six months, a year, two years, three years, four years, five years, or longer).

In one aspect, the invention relates to a method of extending relapse free survival in a cancer patient who had previously undergone cancer therapy (for example, treatment with one or more therapeutic agents, radiation and/or surgery) by administering a therapeutically effective amount of a hedgehog inhibitor to the patient after the cancer therapy has ceased. The hedgehog inhibitor can be administered immediately after cancer therapy has ceased, or there can be a gap in time (e.g., up to about a day, a week, a month, six months, or a year) between the end of cancer therapy and the administration of the hedgehog inhibitor.

In some embodiments, the hedgehog inhibitor is a first line treatment for the cancer, i.e., it is used in a subject who has not been previously administered another drug intended to treat the cancer.

In other embodiments, the hedgehog inhibitor is a second line treatment for the cancer, i.e., it is used in a subject who has been previously administered another drug intended to treat the cancer.

In other embodiments, the hedgehog inhibitor is a third or fourth line treatment for the cancer, i.e., it is used in a subject who has been previously administered two or three other drugs intended to treat the cancer.

In some embodiments, a hedgehog inhibitor is administered to a subject following surgical excision/removal of the cancer.

In some embodiments, a hedgehog inhibitor is administered to a subject before, during, and/or after radiation treatment of the cancer.

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphoblastic leukemia (ALL), acute myeloid leukemia (AML), chronic myelogenous leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. primary myelofibrosis (PMF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenström's macroglobulinemia.

In certain embodiments, the cancer is selected from biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphoblastic leukemia (ALL), acute myeloid leukemia (AML), chronic myelogenous leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), multiple myeloma (MM), myelodysplastic syndrome (MDS), myeloproliferative disorder (MPD) (e.g., polycythemia Vera (PV), essential thrombocytosis (ET), agnogenic myeloid metaplasia (AMM) a.k.a. primary myelofibrosis (PMF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (KA), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma).

In certain embodiments, the cancer is selected from bladder cancer, breast cancer, medulloblastoma, colorectal cancer, head and neck cancer, small cell lung cancer (SCLC), non-small cell lung cancer (NSCLC), acute lymphocytic leukemia (ALL), acute myelocytic leukemia (AML), chronic myelocytic leukemia (CML), chronic lymphocytic leukemia (CLL), Hodgkin lymphoma (HL), non-Hodgkin lymphoma (NHL), multiple myeloma (MM), osteosarcoma, ovarian cancer, pancreatic cancer, prostate cancer, basal cell carcinoma (BCC)) and chondrosarcoma.

In certain embodiments, the cancer is bladder cancer.

In certain embodiments, the cancer is breast cancer.

In certain embodiments, the cancer is medulloblastoma.

In certain embodiments, the cancer is an ovarian cancer, e.g., a platinum-resistant ovarian cancer or serous ovarian cancer.

In certain embodiments, the cancer is colorectal cancer.

In certain embodiments, the cancer is head and neck cancer.

In certain embodiments, the cancer is lung cancer. In certain embodiments, the cancer is small cell lung cancer (SCLC). In certain embodiments, the cancer is non-small cell lung cancer (NSCLC).

In certain embodiments, the cancer is leukemia. In certain embodiments, the cancer is acute lymphocytic leukemia (ALL). In certain embodiments, the cancer is acute myelocytic leukemia (AML). In certain embodiments, the cancer is chronic myelocytic leukemia (CML). In certain embodiments, the cancer is chronic lymphocytic leukemia (CLL).

In certain embodiments, the cancer is lymphoma. In certain embodiments, the cancer is Hodgkin lymphoma (HL). In certain embodiments, the cancer is non-Hodgkin lymphoma (NHL).

In certain embodiments, the cancer is multiple myeloma (MM).

In certain embodiments, the cancer is osteosarcoma.

In certain embodiments, the cancer is ovarian cancer.

In certain embodiments, the cancer is pancreatic cancer.

In certain embodiments, the cancer is prostate cancer.

In certain embodiments, the cancer is basal cell carcinoma (BCC).

In certain embodiments, the cancer is a medulloblastoma.

In certain embodiments, the cancer is chondrosarcoma.

In certain embodiments, the cancer is neuroendocrine cancer.

Neuroendocrine cancers (also known as gastroenteropancreatic tumors or gastroenteropancreatic neuroendocrine cancers), are cancers derived from cells at the interface between the endocrine (hormonal) system and the nervous system. The majority of neuroendocrine cancers fall into two categories: carcinoids and pancreatic endocrine tumors (also known as endocrine pancreatic tumors or islet cell tumors). In addition to the two main categories, other forms of neuroendocrine cancers exist, including neuroendocrine lung tumors, which arise from the respiratory rather than the gastro-entero-pancreatic system. Neuroendocrine cancers can originate from endocrine glands such as the adrenal medulla, the pituitary, and the parathyroids, as well as endocrine islets within the thyroid or the pancreas, and dispersed endocrine cells in the respiratory and gastrointestinal tract.

For example, the cancer treated can be a neuroendocrine cancer chosen from one or more of, e.g., a neuroendocrine cancer of the pancreas, lung, appendix, duodenum, ileum, rectum or small intestine. In other embodiments, the neuroendocrine cancer is chosen from one or more of: a pancreatic endocrine tumor; a neuroendocrine lung tumor; or a neuroendocrine cancer from the adrenal medulla, the pituitary, the parathyroids, thyroid endocrine islets, pancreatic endocrine islets, or dispersed endocrine cells in the respiratory or gastrointestinal tract.

Pancreatic endocrine tumors can secrete biologically active peptides (e.g., hormones) that can cause various symptoms in a subject. Such tumors are referred to functional or secretory tumors. Functional tumors can be classified by the hormone most strongly secreted. Examples of functional pancreatic endocrine tumors include gastrinoma (producing excessive gastrin and causing Zollinger-Ellison Syndrome), insulinoma (producing excessive insulin), glucagonoma (producing excessive glucagon), vasoactive intestinal peptideoma (VIPoma, producing excessive vasoactive intestinal peptide), PPoma (producing excessive pancreatic polypeptide), somatostatinoma (producing excessive somatostatin), watery diarrhea hypokalemia-achlorhydria (WDHA), CRHoma (producing excessive corticotropin-releasing hormones), calcitoninoma (producing excessive calcitonin), GHRHoma (producing excessive growth-hormone-releasing hormone), neurotensinoma (producing excessive neurotensin), ACTHoma (producing excessive adrenocorticotropic hormone), GRFoma (producing excessive growth hormone-releasing factor), and parathyroid hormone-related peptide tumor. In some instances, pancreatic endocrine tumors can arise in subjects who have multiple endocrine neoplasia type 1 (MEN1); such tumors often occur in the pituitary gland or pancreatic islet cells. Pancreatic endocrine tumors that do not secrete peptides (e.g., hormones) are called nonfunctional (or nonsecretory or nonfunctional) tumors.

In other embodiments, the cancer treated is a carcinoid tumor, e.g., a carcinoid neuroendocrine cancer. Carcinoid tumors tend to grow more slowly than pancreatic endocrine tumors. A carcinoid tumor can produce biologically active molecules such as serotonin, a biogenic molecule that causes a specific set of symptoms called carcinoid syndrome. Carcinoid tumors that produce biologically active molecules are often referred to as functional carcinoid tumors, while those that do not are referred to as nonfunctional carcinoid tumors. In some embodiments, the neuroendocrine cancer is a functional carcinoid tumor (e.g., a carcinoid tumor that can produce biologically active molecules such as serotonin). In other embodiments, the neuroendocrine cancer is a non-functional carcinoid tumor. In certain embodiments, the carcinoid tumor is a tumor from the thymus, stomach, small intestine (duodenum, jejunum, ileum), large intestine (cecum, colon), rectal, pancreatic, appendix, ovarian or testicular carcinoid.

Carcinoid tumors can be further classified depending on the point of origin, such as lung, thymus, stomach, small intestine (duodenum, jejunum, ileum), large intestine (cecum, colon), rectum, pancreas, appendix, ovaries and testes. In some embodiments, the neuroendocrine cancer is a http://en.wikipedia.org/wiki/Caarcinoidcarcinoid tumor. In other embodiments, the neuroendocrine cancer is a pancreatic endocrine tumor. In still other embodiments, the neuroendocrine cancer is a neuroendocrine lung tumor. In certain embodiments, the neuroendocrine cancers originate from the adrenal medulla, the pituitary, the parathyroids, thyroid endocrine islets, pancreatic endocrine islets, or dispersed endocrine cells in the respiratory or gastrointestinal tract.

Further examples of neuroendocrine cancers that can be treated include, but are not limited to, medullary carcinoma of the thyroid, Merkel cell cancer (trabecular cancer), small-cell lung cancer (SCLC), large-cell neuroendocrine carcinoma (of the lung), extrapulmonary small cell carcinomas (ESCC or EPSCC), neuroendocrine carcinoma of the cervix, Multiple Endocrine Neoplasia type 1 (MEN-1 or MEN1), Multiple Endocrine Neoplasia type 2 (MEN-2 or MEN2), neurofibromatosis type 1, tuberous sclerosis, von Hippel-Lindau (VHL) disease, neuroblastoma, pheochromocytoma (phaeochromocytoma), paraganglioma, neuroendocrine cancer of the anterior pituitary, and/or Carney's complex.

In certain embodiments, the cancer has a fibrotic component. In one embodiment, the cancer has fibrosis of the bone marrow or a hematopoietic tissue. In certain embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In other embodiments, the bone marrow fibrosis is associated with (e.g., is secondary to) a malignant condition or a condition caused by a clonal proliferative disease. In other embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CML)). In yet other embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

In embodiments where a fibrotic condition of the bone marrow is treated, the hedgehog inhibitor can be administered in combination with an agent chosen from a Jak2 inhibitor (including, but not limited to, INCB018424, XL019, TG101348, or TG101209), an immunomodulator, e.g., an IMID (including, but not limited to thalidomide, lenalidomide, or panolinomide), hydroxyurea, an androgen, erythropoietic stimulating agents, prednisone, danazol, HDAC inhibitors, or other agents or therapeutic modalities (e.g., stem cell transplants, or radiation).

Certain methods of the current invention can be especially effective in treating cancers that respond well to existing chemotherapies, but suffer from a high relapse rate. In these instances, treatment with the hedgehog inhibitor can increase the relapse-free survival time or rate of the patient. The invention also encompasses the use of a therapeutic agent and a hedgehog inhibitor for preparation of one or more medicaments for use in the methods described herein. The invention also relates to the use of a hedgehog inhibitor in the preparation of a medicament for use in the methods described herein. The invention also encompasses the use of a hedgehog inhibitor in the preparation of a medicament for use in a method of treating a cancer patient as described herein.

Multiple tumor types exhibit up-regulation of Hh ligands post chemotherapy and in response to other stress, such as hypoxia. The type of Hh ligand that is up-regulated (i.e., Sonic, Indian and/or Desert) and the degree of up-regulation vary depending upon the tumor type and the therapeutic agent. Without wishing to be bound to any theory, these results suggest that stress (including chemotherapy) induces Hedgehog ligand production in tumor cells as a protective or survival mechanism. The results further suggest that up-regulation of tumor-derived Hh ligand post-chemotherapy can confer upon the surviving cell population a dependency upon the Hh pathway that is important for tumor recurrence, and thus can be susceptible to Hh pathway inhibition.

Thus, an aspect of the invention is a method of treating cancer by determining whether expression of one or more hedgehog ligands has increased during or after chemotherapy, then administering a hedgehog inhibitor. Ligand expression can be measured by detection of a soluble form of the ligand in peripheral blood and/or urine (e.g., by an ELISA assay or radioimmunoassay), in circulating tumor cells (e.g., by a fluorescence-activated cell sorting (FACS) assay, an immunohistochemistry assay, or a reverse transcription polymerase chain reaction (RT-PCR) assay), or in tumor or bone marrow biopsies (e.g., by an immunohistochemistry assay, a RT-PCR assay, or by in situ hybridization). Detection of hedgehog ligand in a given patient tumor could also be assessed in vivo, by systemic administration of a labeled form of an antibody to a hedgehog ligand followed by imaging, similar to detection of PSMA in prostate cancer patients (Bander, N H Nat Clin Pract Urol 2006; 3:216-225). Expression levels in a patient can be measured at least at two time-points to determine of ligand induction has occurred. For example, hedgehog ligand expression can be measured pre- and post-chemotherapy, pre-chemotherapy and at one or more time-points while chemotherapy is ongoing, or at two or more different time-points while chemotherapy is ongoing. If a hedgehog ligand is found to be up-regulated, a hedgehog inhibitor can be administered. Thus, measurement of hedgehog ligand induction in the patient can determine whether the patient receives a hedgehog pathway inhibitor in combination with or following other chemotherapy.

Another aspect of the invention relates to a method of treating cancer in a patient by identifying one or more therapeutic agents that elevate hedgehog ligand expression in the cancer tumor, and administering one or more of the therapeutic agents that elevate hedgehog ligand expression and a hedgehog inhibitor. To determine which therapeutic agents elevate hedgehog expression, tumor cells can be removed from a patient prior to therapy and exposed to a panel of therapeutic agents ex vivo and assayed to measure changes in hedgehog ligand expression (see, e.g., Am. J. Obstet. Gynecol. November 2003, 189(5):1301-7; J. Neurooncol., February 2004, 66(3):365-75). A therapeutic agent that causes an increase in one or more hedgehog ligands is then administered to the patient. A therapeutic agent that causes an increase in one or more hedgehog ligands can be administered alone or in combination with one or more different therapeutic agents that can or can not cause an increase in one or more hedgehog ligands. The hedgehog inhibitor and therapeutic agent can be administered concurrently (i.e., essentially at the same time, or within the same treatment) or sequentially (i.e., one immediately following the other, or alternatively, with a gap in between administration of the two). Treatment with the hedgehog inhibitor can continue after treatment with the therapeutic agent ceases. Thus, the therapeutic agent is chosen based upon its ability to up-regulate hedgehog ligand expression (which, in turn, renders the tumors dependent upon the hedgehog pathway), which can make the tumor susceptible to treatment with a hedgehog inhibitor.

### Combination Therapy

It will be appreciated that the compositions, e.g., one or more hedgehog inhibitors described herein or pharmaceutical compositions thereof, can be administered in combination with one or more additional therapies, e.g., such as radiation therapy, surgery and/or in combination with one or more therapeutic agents, to treat the cancers described herein.

By “in combination” or “in combination with,” it is not intended to imply that the therapy or the therapeutic agents must be administered at the same time and/or formulated for delivery together, although these methods of delivery are within the scope of the invention. The compositions, e.g., one or more hedgehog inhibitors described herein, can be administered concurrently with, prior to, or subsequent to, a cancer therapy (e.g., a primary cancer therapy, e.g., a cancer therapy that includes one or more other additional therapies or therapeutic agents).

In general, each agent will be administered at a dose and/or on a time schedule determined for that agent. In will further be appreciated that the additional therapeutic agent utilized in this combination may be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination are expected to be lower than those utilized individually.

In certain embodiments, the hedgehog inhibitor and the additional therapeutic agent are administered concurrently (i.e., administration of the two agents at the same time or day, or within the same treatment regimen) or sequentially (i.e., administration of one agent over a period of time followed by administration of the other agent for a second period of time, or within different treatment regimens).

In certain embodiments, the hedgehog inhibitor and the additional therapeutic agent are administered concurrently. For example, in certain embodiments, the hedgehog inhibitor and the additional therapeutic agent are administered at the same time. In certain embodiments, the hedgehog inhibitor and the additional therapeutic agent are administered on the same day. In certain embodiments, the hedgehog inhibitor is administered after the additional therapeutic agent on the same day or within the same treatment regimen. In certain embodiments, the hedgehog inhibitor is administered before the additional therapeutic agent on the same day or within the same treatment regimen.

In certain embodiments, a hedgehog inhibitor is concurrently administered with additional therapeutic agent for a period of time, after which point treatment with the additional therapeutic agent is stopped and treatment with the hedgehog inhibitor continues.

In other embodiments, a hedgehog inhibitor is concurrently with the additional therapeutic agent for a period of time, after which point treatment with the hedgehog inhibitor is stopped and treatment with the additional therapeutic agent continues.

In certain embodiments, the hedgehog inhibitor and the additional therapeutic agent are administered sequentially. For example, in certain embodiments, the hedgehog inhibitor is administered after the treatment regimen of the additional therapeutic agent has ceased. In certain embodiments, the additional therapeutic agent is administered after the treatment regimen of the hedgehog inhibitor has ceased.

In yet other embodiments, the hedgehog inhibitor, alone or combination with the therapeutic agent is administered in a therapeutically effective amount, e.g., at a predetermined dosage schedule.

In other embodiments, a hedgehog inhibitor and a therapeutic agent can be used in combination with one or more of other therapeutic agents, radiation, and/or surgical procedures.

Cancer therapies include, but are not limited to, surgery and surgical treatments, radiation therapy, and therapeutic agents (e.g., biotherapeutic agents and chemotherapeutic agents).

In certain embodiments, the cancer treated by the methods described herein can be selected from, for example, medulloblastoma, chondrosarcoma, osteosarcoma, pancreatic cancer, lung cancer (e.g., small cell lung cancer (SCLC) or non-small cell lung cancer (NSCLC)), ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, and prostate cancer.

An example of suitable therapeutics for use in combination with one or more hedgehog inhibitors for treatment of medulloblastoma includes, but is not limited to, a chemotherapeutic agent (e.g., lomustine, cisplatin, carboplatin, vincristine, and cyclophosphamide), radiation therapy, surgery, and a combination thereof.

An example of suitable therapeutics for use in combination with one or more hedgehog inhibitors for treatment of chondrosarcoma includes, but is not limited to, a chemotherapeutic agent (e.g., trabectedin), radiation therapy (e.g., proton therapy), surgery, and a combination thereof.

An example of suitable therapeutics for use in combination with one or more hedgehog inhibitors for treatment of osteosarcoma includes, but is not limited to, a chemotherapeutic agent (e.g., methotrexate (e.g., alone or in combination with leucovorin rescue), cisplatin, adriamycin, ifosfamide (e.g., alone or in combination with mesna), BCG (Bacillus Calmette-Guerin), etoposide, muramyl tri-peptite (MTP)), radiation therapy, surgery, and a combination thereof.

An example of suitable therapeutics for use in combination with one or more hedgehog inhibitors for treatment of pancreatic cancer includes, but is not limited to, a chemotherapeutic agent, e.g., paclitaxel or a paclitaxel agent (e.g., a paclitaxel formulation such as TAXOL®, an albumin-stabilized nanoparticle paclitaxel formulation (e.g., ABRAXANE®) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE®), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., RO5126766); Notch signaling inhibitor (e.g., MK0752); monoclonal antibody-antibody fusion protein (e.g., L191L2); curcumin; HSP90 inhibitor (e.g., IPI-493, IPI-504, tanespimycin, STA-9090); rIL-2; denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., RO4929097); ribonucleotide reductase inhibitor (e.g., 3-AP); immunotoxin (e.g., HuC242-DM4); PARP inhibitor (e.g., KU-0059436, veliparib); CTLA-4 inhibitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof. In certain embodiments, a combination of paclitaxel or a paclitaxel agent, and gemcitabine can be used with the pharmaceutical compositions of the invention.

An example of suitable therapeutics for use in combination with one or more hedgehog inhibitors for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., IPI-493, IPI-504, tanespimycin, STA-9090); mTOR inhibitor (e.g., everolimus); Ep-CAM-/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

An example of suitable therapeutics for use in combination with one or more hedgehog inhibitors for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, RO5083945), MET inhibitor (e.g., PF-02341066, ARQ 197), PI3K kinase inhibitor (e.g., XL147, GDC-0941), Raf/MEK dual kinase inhibitor (e.g., RO5126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., IPI-493, IPI-504, tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fus1, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination with one or more hedgehog inhibitors for treatment of ovarian cancer includes, but is not limited to, a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agent (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., RO4929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy; and combinations thereof.

An example of suitable therapeutics for use in combination with one or more hedgehog inhibitors for treatment of chronic myelogenous leukemia (AML) according to the invention includes, but is not limited to, a chemotherapeutic (e.g., cytarabine (Ara-C), hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib, RGB-286638)), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., IPI-493, IPI-504, tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., RO5045337), Aurora B kinase inhibitor (e.g., TAK-901), radioimmunotherapy (e.g., actinium-225-labeled anti-CD33 antibody HuM195), Hedgehog inhibitor (e.g., PF-04449913), STATS inhibitor (e.g., OPB-31121), KB004, cancer vaccine (e.g., AG858), bone marrow transplantation, stem cell transplantation, radiation therapy, and combinations thereof. In one embodiment, the AML treatment includes one or more hedgehog inhibitors in combination with high dose Ara-C (HDAC). An exemplary HDAC treatment includes high-dose cytarabine at a dose of 3000 mg/m2 every 12 (q12) hours on days 1, 3 and 5 (total of 6 doses).

An example of suitable therapeutics for use in combination with one or more hedgehog inhibitors for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., ABT-263), immunotherapy (e.g., allogeneic CD4+ memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., RO5045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination with one or more hedgehog inhibitors for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., RO5045337), immunotoxin (e.g., CAT-8015, DT2219ARL), HDAC inhibitor (e.g., JNJ-26481585), JVRS-100, paclitaxel or a paclitaxel agent, STATS inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination with one or more hedgehog inhibitors for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT3881L3 fusion protein, HDAC inhibitor (e.g., vorinostat, LBH589), plerixafor, mTOR inhibitor (e.g., everolimus), SRC inhibitor (e.g., dasatinib), HSP90 inhibitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., RO5045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of suitable therapeutics for use in combination with one or more hedgehog inhibitors for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STATS inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of suitable therapeutics for use in combination with one or more hedgehog inhibitors for treatment of head and neck cancer includes, but is not limited to, a chemotherapeutic (e.g., paclitaxel or a paclitaxel agent, carboplatin, docetaxel, amifostine, cisplantin, oxaliplatin, docetaxel), tyrosine kinase inhibitors (e.g., EGFR inhibitor (e.g., erlotinib, gefitinib, icotinib, cetuximab, panitumumab, zalutumumab, nimotuzumab, necitumumab, matuzumab, cetuximab), dual inhibitor (e.g., lapatinib, neratinib, vandetanib, BIBW 2992, multikinase inhibitor (e.g., XL-647)), VEGF inhibitor (e.g., bevacizumab), reovirus, radiation therapy, surgery, and a combination thereof.

An example of suitable therapeutics for use in combination with one or more hedgehog inhibitors for treatment of prostate cancer includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

In some embodiments, the one or more hedgehog inhibitors described herein is used in combination with a mTOR inhibitor, e.g., one or more mTOR inhibitors chosen from one or more of rapamycin, temsirolimus (TORISEL®), everolimus (RAD001, AFINITOR®), ridaforolimus, AP23573, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, or PKI-587. In one embodiment, the mTOR inhibitor inhibits TORC1 and TORC2. Examples of TORC1 and TORC2 dual inhibitors include, e.g., OSI-027, XL765, Palomid 529, and INK128.

In some embodiments, the one or more hedgehog inhibitors described herein is used in combination with an inhibitor of insulin-like growth factor receptor (IGF-1R), e.g., BMS-536924, GSK1904529A, AMG 479, MK-0646, cixutumumab, OSI 906, figitumumab (CP-751,871), or BIIB022.

In some embodiments, the one or more hedgehog inhibitors described herein is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In some embodiments, the one or more hedgehog inhibitors described herein is used in combination with folfirinox comprising oxaliplatin 85 mg/m2 and irinotecan 180 mg/m2 plus leucovorin 400 mg/m2 followed by bolus fluorouracil (5-FU) 400 mg/m2 on day 1, then 5-FU 2,400 mg/m2 as a 46-hour continuous infusion.

In some embodiments, the one or more hedgehog inhibitors described herein is used in combination with a PI3K inhibitor. In one embodiment, the PI3K inhibitor is an inhibitor of delta and gamma isoforms of PI3K. Exemplary PI3K inhibitors that can be used in combination are described in, e.g., WO 09/088,990; WO 09/088,086; WO 2011/008302; WO 2010/036380; WO 2010/006086, WO 09/114,870, WO 05/113556; US 2009/0312310, US 2011/0046165. Additional PI3K inhibitors that can be used in combination with the hedgehog inhibitors, include but are not limited to, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof.

In some embodiments, the one or more hedgehog inhibitors described herein is used in combination with a HSP90 inhibitor. The HSP90 inhibitor can be a geldanamycin derivative, e.g., a benzoquinone or hydroquinone ansamycin HSP90 inhibitor (e.g., IPI-493 and/or IPI-504). Non-limiting examples of HSP90 inhibitors include IPI-493, IPI-504, 17-AAG (also known as tanespimycin or CNF-1010), BIIB-021 (CNF-2024), BIIB-028, AUY-922 (also known as VER-49009), SNX-5422, STA-9090, AT-13387, XL-888, MPC-3100, CU-0305, 17-DMAG, CNF-1010, Macbecin (e.g., Macbecin I, Macbecin II), CCT-018159, CCT-129397, PU-H71, or PF-04928473 (SNX-2112).

In some embodiments, the one or more hedgehog inhibitors described herein is administered in combination with a BRAF inhibitor, e.g., GSK2118436, RG7204, PLX4032, GDC-0879, PLX4720, and sorafenib tosylate (Bay 43-9006).

In some embodiments, the one or more hedgehog inhibitors described herein is administered in combination with a MEK inhibitor, e.g., ARRY-142886, GSK1120212, RDEA436, RDEA119/BAY 869766, AS703026, AZD6244 (selumetinib), BIX 02188, BIX 02189, CI-1040 (PD184352), PD0325901, PD98059, and U0126.

In some embodiments, the one or more hedgehog inhibitors described herein is administered in combination with a JAK2 inhibitor, e.g., CEP-701, INCB 18424, CP-690550 (tasocitinib).

In one embodiment, the second agent is a taxane, e.g. paclitaxel or a formulation thereof (e.g., albumin-bound paclitaxel (ABRAXANE®), nab-paclitaxel), docetaxel (e.g., as an injectable Docetaxel (Taxotere®)), or taxol).

In some embodiments, the one or more hedgehog inhibitors described herein is administered in combination with paclitaxel or a paclitaxel agent, e.g., TAXOL®, protein-bound paclitaxel (e.g., ABRAXANE®). A “paclitaxel agent” as used herein refers to a formulation of paclitaxel (e.g., for example, TAXOL®) or a paclitaxel equivalent (e.g., for example, a prodrug of paclitaxel). Exemplary paclitaxel equivalents include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX®, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel 2-glucopyranosyl succinate, see Liu et al., Bioorganic & Medicinal Chemistry Letters (2007) 17:617-620). In certain embodiments, the paclitaxel agent is a paclitaxel equivalent. In certain embodiments, the paclitaxel equivalent is ABRAXANE®.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner as disclosed herein include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

Pharmaceutical Compositions

To practice the methods of the invention, the hedgehog inhibitor and/or the therapeutic agent can be delivered in the form of pharmaceutically acceptable compositions which comprise a therapeutically-effective amount of one or more hedgehog inhibitors and/or one or more therapeutic agent formulated together with one or more pharmaceutically acceptable excipients. In some instances, the hedgehog inhibitor and the therapeutic agent are administered in separate pharmaceutical compositions and can (e.g., because of different physical and/or chemical characteristics) be administered by different routes (e.g., one therapeutic is administered orally, while the other is administered intravenously). In other instances, the hedgehog inhibitor and the therapeutic agent can be administered separately, but via the same route (e.g., both orally or both intravenously). In still other instances, the hedgehog inhibitor and the therapeutic agent can be administered in the same pharmaceutical composition.

Pharmaceutical compositions can be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; pulmonarily; or nasally.

Examples of suitable aqueous and nonaqueous carriers which can be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds of the present invention can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Methods of preparing these formulations or compositions include the step of bringing into association the hedgehog inhibitor and/or the therapeutic agent with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The hedgehog inhibitors and the therapeutic agents of the present invention can be given per se or as a pharmaceutical composition containing, for example, about 0.1 to 99%, or about 10 to 50%, or about 10 to 40%, or about 10 to 30%, or about 10 to 20%, or about 10 to 15% of active ingredient in combination with a pharmaceutically acceptable carrier. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a hedgehog inhibitor and/or a therapeutic agent will be that amount of the compound which is the lowest dose effective to produce a therapeutic effect. Such an effective dose will generally depend upon the factors described above. Generally, oral, intravenous and subcutaneous doses of the compounds of the present invention for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to about 500 mg per day, or about 0.1 mg to about 500 mg per day.

The subject receiving this treatment is any animal in need, including primates, in particular humans, equines, cattle, swine, sheep, poultry, dogs, cats, mice and rats.

The compounds can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

Since the hedgehog inhibitors are administered in combination with other treatments (such as additional therapeutic agents, radiation or surgery) the doses of each agent or therapy can be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day. The determination of the mode of administration and the correct dosage is well within the knowledge of the skilled clinician.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Characterization of IPI-926

Assays to ascertain the activity of hedgehog inhibitors, including IPI-926, in cancer cells are described in, for example, US 2009/0181997 by Grayzel et al.; US 2010/0144775; US 2010/0222287; US 2010/0297118; and PCT/U52010/057534, published as WO 2011/063309, on May 26, 2011, the entire contents of the aforesaid applications are incorporated herein by reference.

An exemplary assay to evaluate the activity of a hedgehog inhibitor is as follows. C3H10T1/2 cells differentiate into osteoblasts when contacted with the sonic hedgehog peptide (Shh-N). Upon differentiation, these osteoblasts produce high levels of alkaline phosphatase (AP) which can be measured in an enzymatic assay (Nakamura et al., 1997 BBRC 237: 465). Compounds that block the differentiation of C3H10T1/2 into osteoblasts (a Shh dependent event) can therefore be identified by a reduction in AP production (van der Horst et al., 2003 Bone 33: 899). The assay details are described below. Additional assays to ascertained the activity of hedgehog inhibitors, including IPI-926, are described in US 2009/0181997 by Grayzel et al., U.S. Ser. Nos. 61/327,373 and 61/331,365, filed on Apr. 23, 2010 and May 4, 2010, respectively; the entire contents of the aforesaid applications are incorporated herein by reference.

### Cell Culture

Mouse embryonic mesoderm fibroblasts C3H10T1/2 cells (obtained from ATCC) were cultured in Basal MEM Media (Gibco/Invitrogen) supplemented with 10% heat inactivated FBS (Hyclone), 50 units/ml penicillin and 50 ug/ml streptomycin (Gibco/Invitrogen) at 37° C. with 5% CO₂ in air atmosphere.

### Alkaline Phosphatase Assay

C3H10T1/2 cells were plated in 96 wells with a density of 8×10³ cells/well. Cells were grown to confluence (72 hrs.). After sonic hedgehog (250 ng/ml) and/or compound treatment, the cells were lysed in 110 μL of lysis buffer (50 mM Tris pH 7.4, 0.1% TritonX100), plates were sonicated and lysates spun through 0.2 μm PVDF plates (Corning). 40 μL of lysates was assayed for AP activity in alkaline buffer solution (Sigma) containing 1 mg/ml p-Nitrophenyl Phosphate. After incubating for 30 min at 37° C., the plates were read on an Envision plate reader at 405 nm. Total protein was quantified with a BCA protein assay kit from Pierce according to manufacturer's instructions. AP activity was normalized against total protein. Using the above-described assay, IPI-926 (HCl salt) was shown to be an antagonist of the hedgehog pathway with an IC₅₀ less than 20 nM.

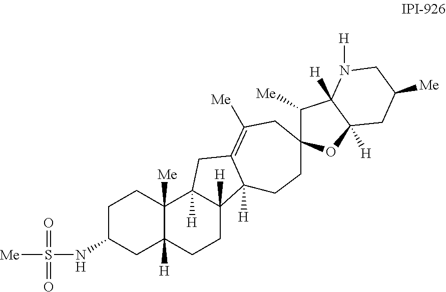

### Example 2

### Evaluation of Ciliary Components as Predictive Markers of Hedgehog Responsiveness

This Example describes a correlation between the presence of cilia in multiple tumor cell lines and hedgehog (Hh) responsiveness.

Several reports show that primary cilia is necessary for Hedgehog signaling. Mutations that affect the assembly or maintenance of cilia have been shown to cause defects in activation of the Hh pathway. For example, Huangfu, D. et al. (2003) Nature 426:83-87 have shown that mutations in intraflagellar transport proteins (IFTs) and KiF3a abrogated hedgehog signaling and resulted in loss of ventral neural cell types. Hedgehog pathway activation depends on the proper localization of hedgehog signaling components, for example, dynamic movement of the hedgehog receptors, patched (Ptch) and smoothened (Smo) into and out of the cilium, activation of Gli1 and/or Gli-2, and processing of Gli3 from activator to repressor (Zaghloul (2009) J. Clin Inv. 119(3): 428-437). Some reports have shown the selective translocation of intracellular Smo to the primary cilium in response to modulation of the hedgehog pathway. Some hedgehog inhibitors, e.g., GDC-0449, are believed to reduce or block movement of Smo to the cilium. Other hedgehog inhibitors can reduce hedgehog signaling, while allowing (or even promoting) Smo to localize to the cilium. For example, some reports have shown that cyclopamine promotes Smo accumulation at the primary cilium.

The experiments described herein demonstrate a correlation between the presence of cilia in several tumor cell lines and hedgehog signaling capability. Several tumor cell lines were tested for Hh pathway responsiveness and for the presence or absence of cilia or vimentin immunofluorescence (IF). Hh pathway responsiveness was evaluated by detecting increases in hGli-1 mRNA expression in response to Sonic Hedgehog stimulation) in a control sample (C), sample treated with Sonic Hedgehog (SHh), and sample treated with SHh and 500 nM of the hedgehog inhibitor IPI-926 (926). Procedures for RNA extraction and RT-PCR detection were performed by standard methods. Briefly, total RNA was extracted from all tumors using a standard RNEASY® Mini Kit (Qiagen). Next, the RNA was converted to cDNA and 50 ng cDNA was used in each reaction/sample for qRT-PCR analysis of the expression of human Gli1. Samples were tested in duplicate. Cilia and Vimentin IF was performed using standard immunofluorescent methods. Cilia were detected on cells using immunofluorescence (IF) with antibodies specific for acetylated-tubulin obtained from Sigma Aldrich. Cells were co-stained with antibodies to the centriole-specific protein pericentrin.

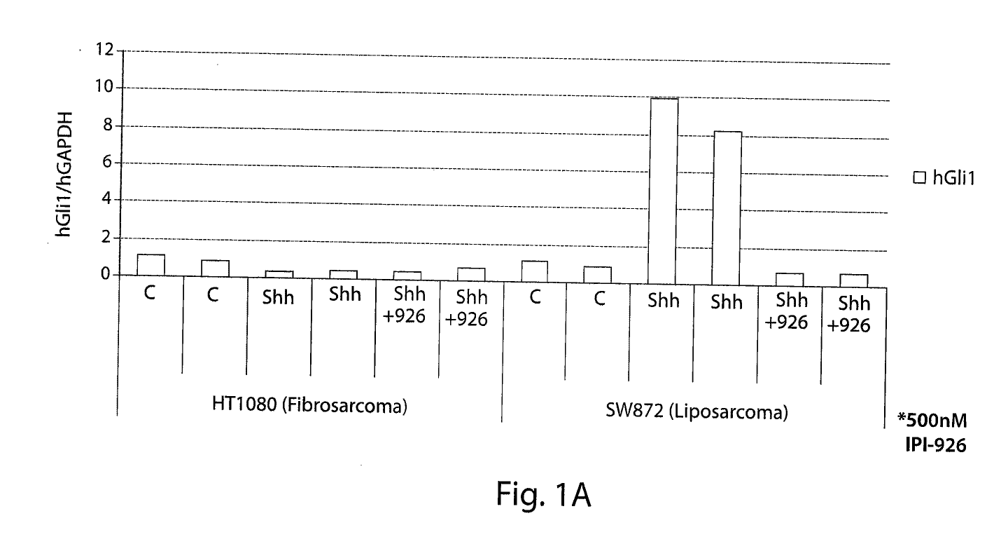

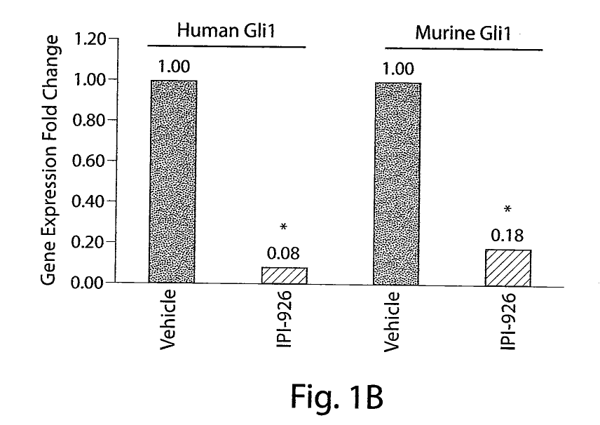

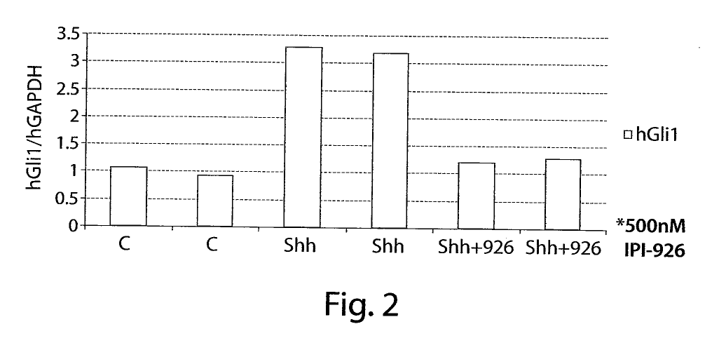

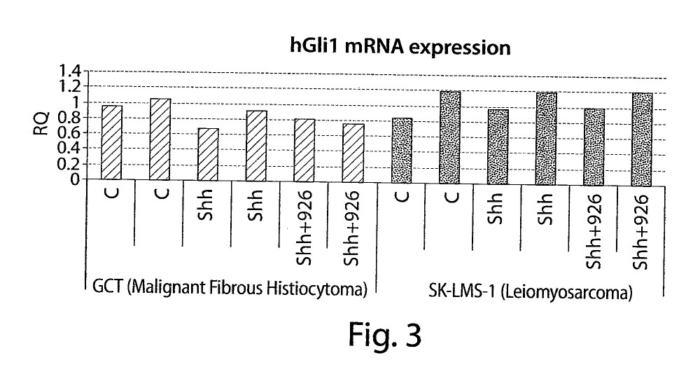

Hh pathway modulation measured after growing cells in low serum and treating with SHh ligand in the presence or absence of IPI-926. Cell lines were cultured under the following conditions. SW872 Liposarcoma cells (shown as FIGS. 1A-1B) were grown to confluency in normal serum media, starved overnight in low serum media, and treated 72 hours in low serum media; MG-63 osteosarcoma cells (shown in FIG. 2) were grown to confluency in normal serum media, starved for 5 days in low serum media, and treated 48 hours in low serum media; GCT (malignant fibrous histiocytoma) and SK-LMS-1 (Leiomyosarcoma) cell lines (shown as FIG. 3) were grown to confluency in normal serum media, starved overnight in low serum media, treated for 48 hours or 72 hours in low serum media; and Synovial Sarcoma SW982 cells were grown to confluency in normal serum media, treated for 48 hr in normal serum media (10% FBS), or starved overnight in low serum media, and treated 48 h in low serum media (0.5% FBS). Samples were tested in duplicate.

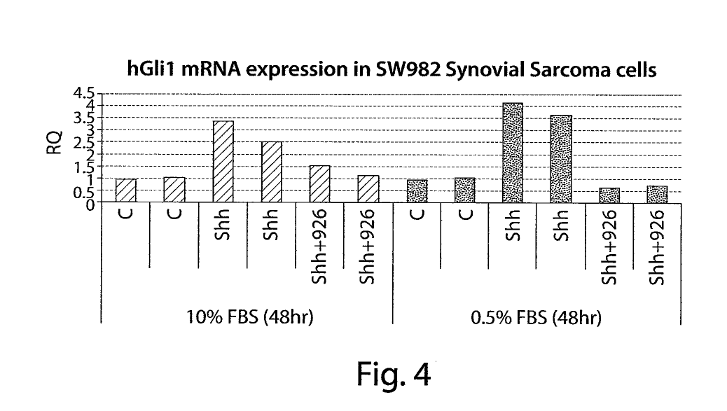

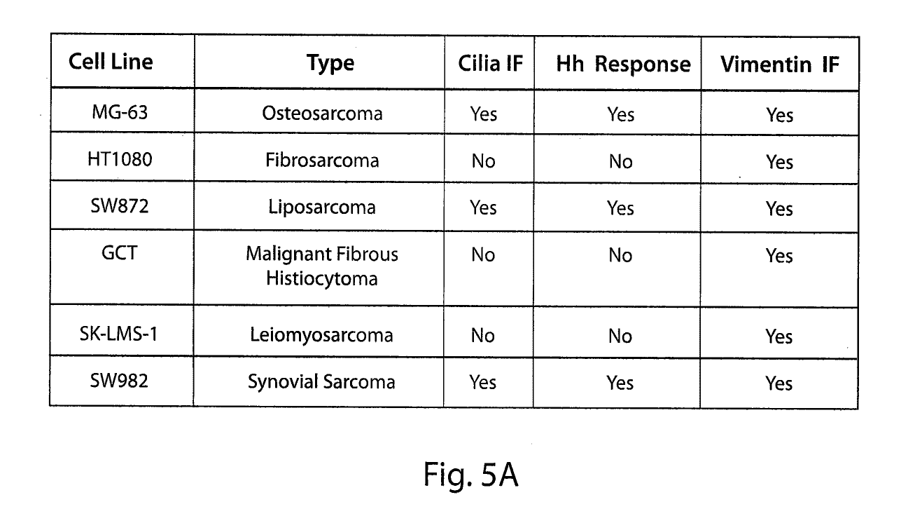

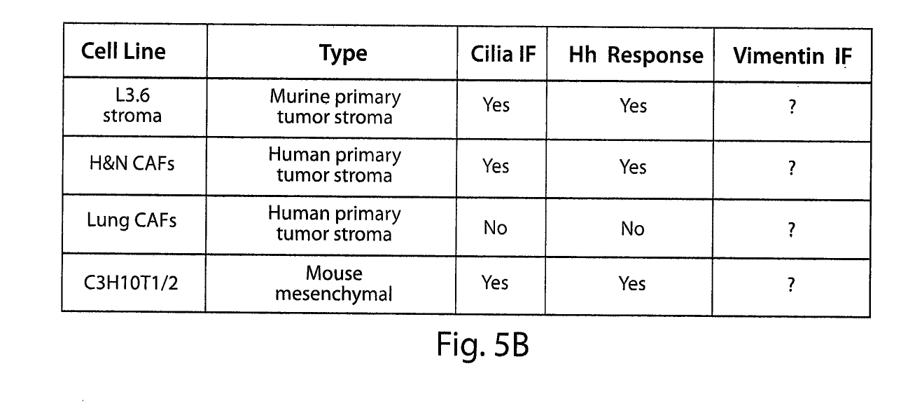

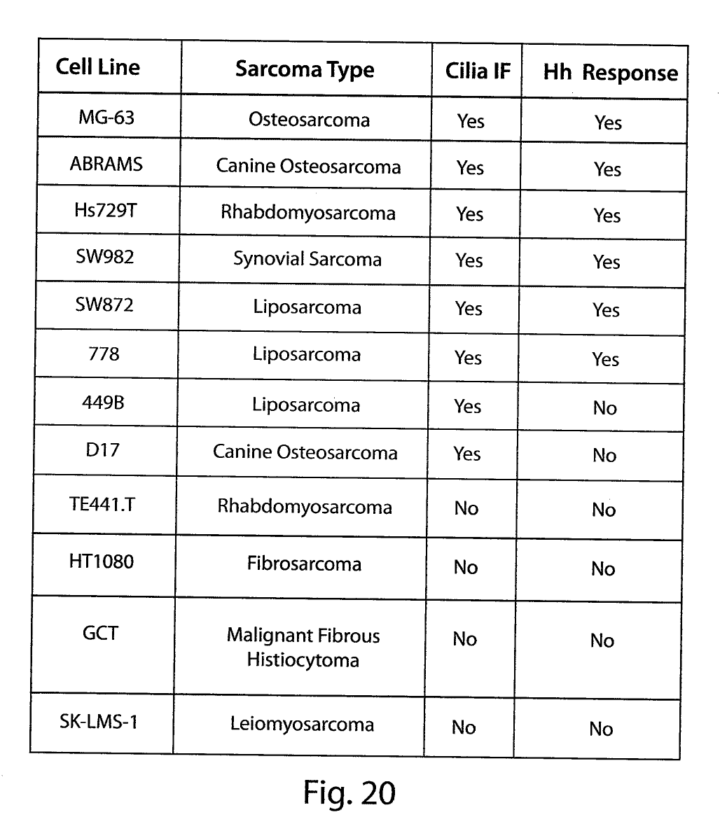

The results from the hedgehog responsiveness assays are shown in FIGS. 1-4, and the correlation between Hh responsiveness and ciliary levels are summarized in FIGS. 5A-5B and 20. FIG. 1 shows upregulated Gli-1 mRNA expression in SW872 liposarcoma cell lines treated with SHh, which effect is blocked by the addition of 500 nM IPI-926. Similar Hh responsiveness is detected in MG-63 osteosarcoma cells (FIG. 2 after prolonged serum starvation) and SW982 synovial sarcoma cell lines (FIG. 4). In contrast, GCT and SK-LMS-1 sarcomas cells were not Hh pathway responsive (FIG. 3). All hedgehog responsive cells tested have cilia IF, whereas cells not responsive to the Hh pathway did not have detectable cilia levels. The results are summarized in FIGS. 5A-5B. Vimentin IF is detected consistently in all samples tested.

FIG. 20 provides another table showing the association of primary cilia with responsiveness to hedgehog inhibition in additional cell lines.

An association between cilia IF and Hh responsiveness was detected in many sarcoma cell lines tested in vitro (FIGS. 5A-5B and 20). More specifically, the following cell lines showed cilia IF and Hh responsiveness: Osteosarcoma cells (e.g., MG-63, ABRAMS); liposarcoma cells (e.g., SW872, 778); synovial sarcoma (SW982); and Hs729T rhabomyosarcoma. Other cell lines showed a similar association, e.g., C3H10T1/2 mouse mesenchymal cells, murine and human primary tumor stromal cells (L3.6 stroma and H&N CAFs) showed cilia IF and Hh pathway responsiveness.

Several Hh non-responsive cells (e.g., GCT and SK-LMS-1 sarcomas cells, TE441.T rhabdomyosarcoma, HT1080 fibrosarcoma, GCT malignant fibrous histiocytoma, SK-LMS-1 leiomyosarcoma, and lung human primary tumor stroma cells (CAFs)) were cilia negative. Two cell lines showed cilia IF but were not responsive to Hh signaling (449B liposarcoma and D17 canine osteosarcoma). Thus, an association is shown herein between the presence of cilia IF and Hh responsiveness in many tumor cell lines.

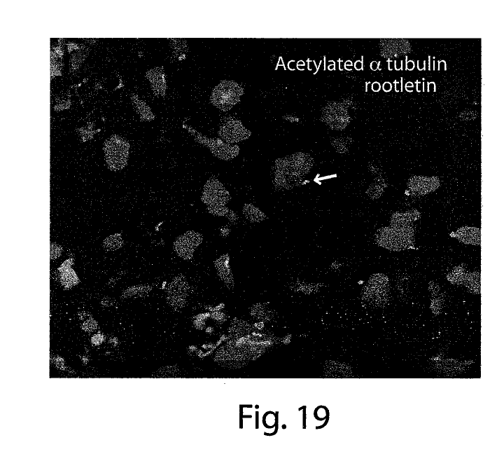

FIG. 19 is a micrograph depicting tumor cells stained for cilia. A human bone and cartilage tumor tissue array (Biomax™) was stained for cilia by immunofluorescence. The majority of the cilia (white arrow) were found on the chondrosarcoma tumor cells.

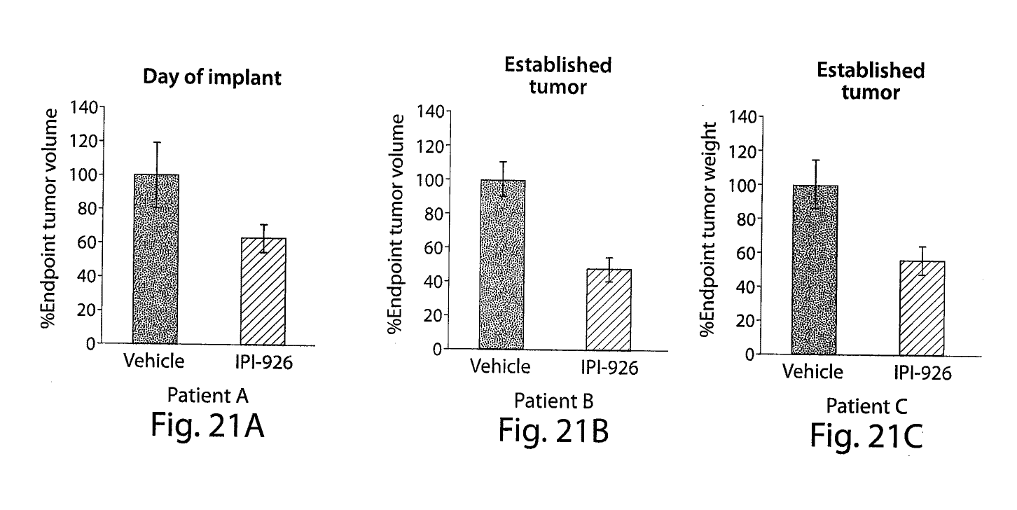

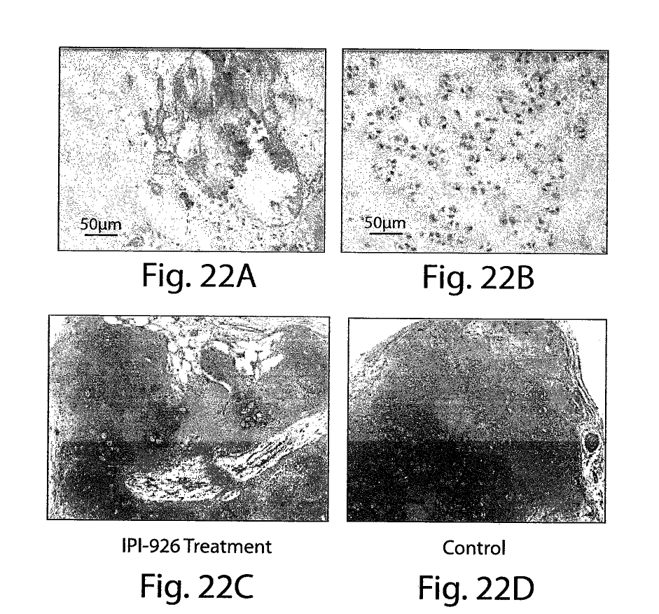

FIGS. 21A-21C show that IPI-926 inhibits tumor growth in multiple patient derived tumor xenograft models. Mice received daily treatment of IPI-926, M-F, for 6-10 weeks (n=10-15 per group). The range of tumor growth inhibition is 33-52%, with a mean of 43%.

The observed correlation between presence of cilia and Hh responsiveness suggest that evaluation of cilia expression can be used as a biomarker for response to Hh pathway activation and inhibition.

### Example 3

### Immunofluorescence of Primary Cilia on Tumor Tissue Sections

This Examples provides protocols for immunofluorescent (IF) detection of cilia in tumor sections.

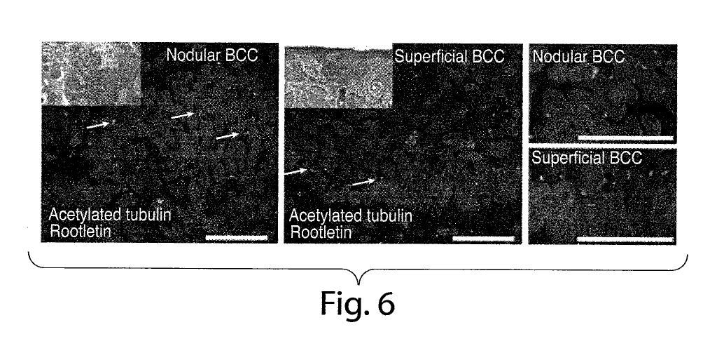

Cilia were detected on sections from Basal Cell Carcinoma (BCC) blocks using immunofluorescence (IF) with antibodies specific for acetylated-tubulin. The antibodies are commercially available from Sigma. Five out of eight BCC specimens were cilia-positive. An exemplary immunohistochemical stain is shown in FIG. 6. Nodular and superficial BCC were immunostained using antibodies specific for acetylated-tubulin. Detectable tubulin protein was detected in the BCC tissue sections examined.

Formalin fixed, paraffin embedded (FFPE) tumor or tissue specimens were sectioned to 5 micron thickness and placed on glass slides. Common antigen retrieval buffers (including DAKO, DIVA, Citrate pH 6.0 and EDTA antigen retrieval buffers) were applied and incubated for twenty minutes in a pressure cooker. After blocking, primary antibodies to acetylated tubulin and/or pericentrin were applied with fluorescently-labeled secondary antibodies used for detection.

Sarcoma tissue microarrays, including multiple sarcoma types and, when available blocks of human sarcomas from collaborators can be used for detection of cilia by IF to determine the prevalence of cilia in multiple tumor types. Primary tumors are relevant for these analyses and can be more accurate than cell lines for determining prevalence and extent of cilia on sarcoma tumor tissues. IF method for acetylated-alpha-tubulin can be performed as described.

In addition, primary osteosarcoma tumors from dogs can be examined. These tumor samples are obtained before and after dogs are treated with IPI-926. The canine cilia can be detected by the same IF method for acetylated-tubulin as described.

Examination of multiple tumor types for the presence of cilia, both in native tumors and in tumor samples obtained after tumor reduction, such as after chemotherapy treatment, can be performed using the protocols described herein.

### Example 4

### Biomarkers for Evaluating Hedgehog Pathway Inhibitors

This Example describes the identification and assays for testing predictive biomarkers for evaluating Hedgehog (Hh) pathway inhibitors.

These predictive biomarkers provide a baseline molecular characteristic of the tumor (e.g. SHh and Gli1 levels) or of the patient (e.g. germline DNA SNP) that is associated with clinical outcome. These markers can be prognostic, predictive of clinical benefit or both. In one embodiment, the predictive biomarker can be used to identify a patient subpopulation (e.g., a patient having a pancreatic cancer) likely to benefit from IPI-926 treatment. At least two assays can be developed. In one embodiment, the assays detect levels of hedgehog ligands by immunohistochemistry (IHC) and ELISA. In other embodiments, genomic and proteomic assays can be developed.

Exemplary Hh biomarkers that can be evaluated include:

Markers of Hh pathway Activation
(i) Ligands (SHh, IHh & DHh) in tumor tissue and serum by IHC, ELISA and/or Mass spectrometry;
(ii) Gli1, Gli2 and Gli3 levels by IHC;
(iii) SHh SNPs in germline DNA;
(iv) tumor architecture: desmoplasia, microvascular density and pericytes, by histopathology analysis and related markers, e.g. CD31 and Meca32;
(v) Reactive stroma molecular signature by RT-PCR.

### Genomic Changes by DNA Sequencing

(i) KRAS, TGFβ-SMADs, p53, cyclin D1 and Gli1 amplification, and additional genes involved in cancer;

(ii) Hh pathway mutations, e.g., SMO, PTCH, SUFU, GLI1, GLI3, BOC and additional members;

(iii) Markers of Epithelial to Mesenchymal Transition (EMT) and additional tissue markers by IHC, e.g. snail, twist, vimentin, slug, cadherins, SPARC;

(iv) Gemcitabine markers of response, including but not restricted to genes involved in the metabolism, transport and DNA repair mechanisms, e.g. SLC29A1, SLC28A1, SLC28A3, CDA, NT5C, DCK, UMP/CMP kinase, RRM1, RRM2, Nucleoside diphosphate kinase, HuR.

For the development of IHC assays and serum proteomics, the following three-step study can be developed:

### 1. Assay Development

Materials and Methods: ˜20 tumor (FFPE and frozen samples)/blood matched pairs from pancreatic cancer patients (and some normal pancreatic tissue and blood controls) can be evaluated. A serum SHh ELISA and IHC assays for Hh ligand, Gli1/2, SPARC, desmoplasia, microvascular density and EMT markers (snail, twist etc) can be developed.

More specifically, the tumor FFPE samples can be analyzed by IHC for detection of Hh ligand, Gli1/2, SPARC, desmoplasia, microvascular density, SMA, genotypic and sequencing. Freshly frozen samples can be evaluated for DNA sequencing of Hh pathway and oncogene genotyping (e.g., to detect SHh SNPs). The frozen samples can also be assayed for evaluate expression profiling (e.g., reactive stroma signature). The IHC assays are semi-quantitative/quantitative; slides can be scored and analyzed using the Aperio software, specifically employing Genie™ histology pattern recognition software.

Blood samples can be analyzed as follows. Blood plasma and serum can be evaluated to detect Hh ligands. Genomic DNA can be obtained from peripheral blood lymphocytes (PBLs) and evaluated using the techniques described herein.

At least the following parameters can be evaluated:

(i) the correlation between SHh expression in tissue biopsies (detected by IHC) and the presence of circulating SHh;

(ii) the prevalence and dynamic range of marker expression in pancreatic tumor tissues, i.e., Gli1 IHC;

(iii) the relationship between markers of pathway activation (e.g., SHh, Gli1, WIF, cilia) and desmoplasia, MVD, SPARC;

(iv) the correlation between mutant KRAS (and other oncogenic changes) and SHh/Gli1 and additional markers or pathway activation.

As an example, the expression of Sonic hedgehog (SHh) in tissue biopsies was examined. SHh was found to be widely expressed in primary tumors and xenograft models (FIG. 7). More specifically, elevated expression of SHh was found in pancreatic ductal adenocarcinoma (70% positive), colon adenocarcinoma (84% positive), ovarian cystadenocarcinoma (44% positive) and prostate adenocarcinoma (77% positive).

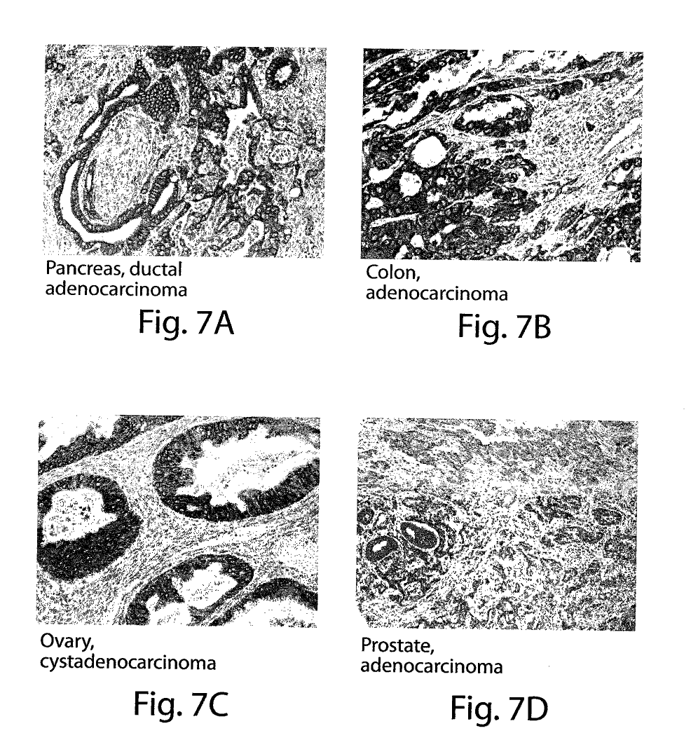

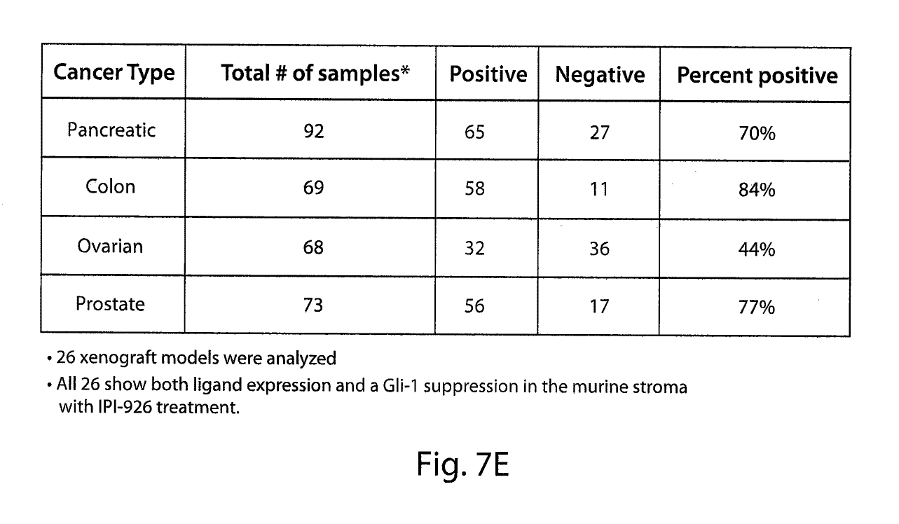

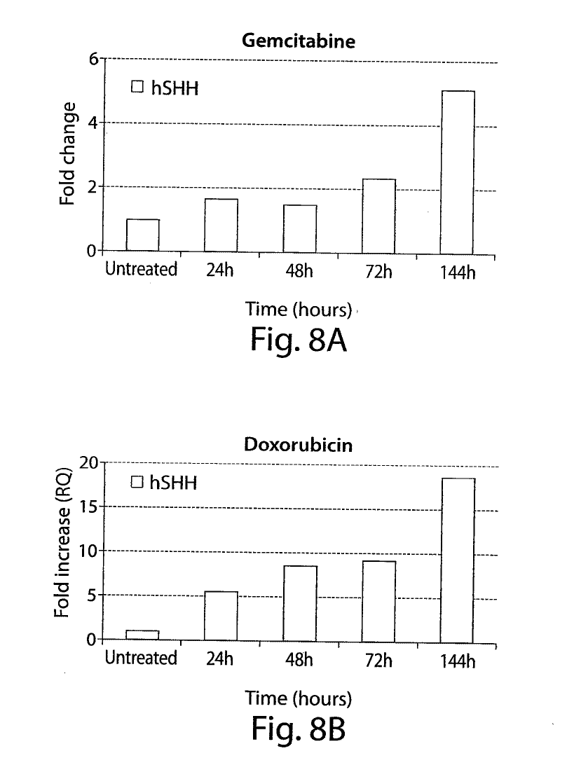

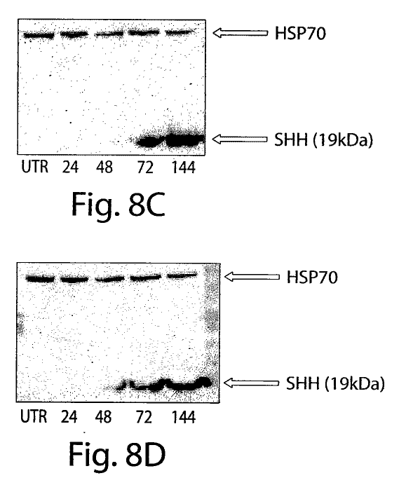

In yet another example, chemotherapy increases hedgehog ligand expression in several in vitro cell cultures tested, including bladder cancer cells. FIGS. 8A-8B show a timecourse of increased human SHh expression in bladder cells treated with gemcitabine and doxorubicin. A consistent increase in human SHh expression is detected starting at 24 hours, 48 hours and 72 hours, with the highest level detected after 144 hours of treatment. The lower panels (FIGS. 8C-8D) show the corresponding protein levels detected by Western blots.

Elevated Hh ligand expression is associated with several tumors and is upregulated upon chemotherapy, and thus provides a useful biomarker for diagnosis and therapy with a hedgehog inhibitor, e.g., IPI-926.

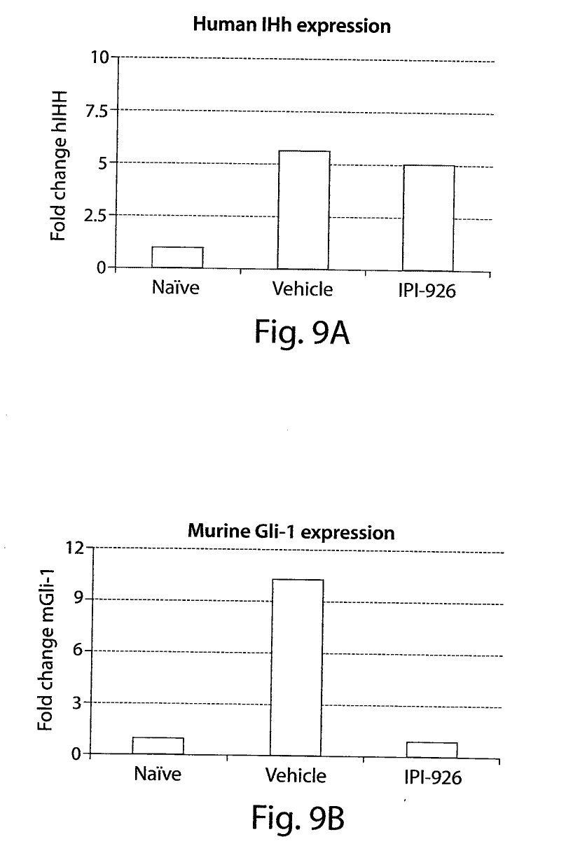

Detection of Gli-1 levels provides for an additional biomarker for diagnosis and evaluating therapy with a hedgehog inhibitor. Gli-1 suppression was detected in the murine stroma after IPI-926 treatment. For example, FIGS. 9A and 9B show upregulation of expression of human IHh ligand in after chemotherapy of LX-22 tumors, whereas expression of murine Gli-1 in the tumor stroma was decreased after chemotherapy. Similar finding are disclosed in PCT/US2010/057534, published as WO 2011/063309, on May 26, 2011.

Thus, elevation of one or more hedgehog ligands, e.g., SHh, and/or suppression of markers, such as Gli-1, can be used as biomarkers for therapy with a chemotherapeutic agent and/or a hedgehog inhibitor, e.g., IPI-926. Changes in Gli-1 levels can be detected in the tumor or stroma on or after treatment with a Hh inhibitor.

### 2. Tissue Microarray (TMA) Analysis

Goal: Using assays developed in step 1, the biomarker prevalence and correlation with molecular and clinical characteristics of pancreatic tumors in pancreatic cancer TMAs can be assessed. The evaluation of desmoplasia in paired primary tumor/liver metastasis TMA can be emphasized.
Materials and Methods: Acquire pancreatic cancer TMAs with the associated molecular and clinical annotation. The number of cases in the TMAs can be approximately two hundred. The assays can be carried out and scored. Statistical analysis can be performed using single and multiple predictor statistical models.

### 3. Retrospective Analysis of Hedgehog Clinical Trials (IPI-926)

Goal: To explore the association between efficacy and putative predictive biomarkers for pancreatic clinical trials.
Materials and Methods: Samples from pancreatic clinical trials using a hedgehog inhibitor can be assayed in order to correlate these putative markers with IPI-926 efficacy data. Where pre- and on-treatment matched samples are available, the effect of IPI-926 on tumor-associated stroma can be addressed.

### Example 5

### Biomarkers for Evaluating Effects of Hedgehog Pathway Inhibitors on Tumor Stroma

Several lines of evidence suggest that hedgehog inhibitors, such as IPI-926, affect tumor stroma, and enable more chemotherapy to access tumor. This conclusion is supported by the following: morphological analysis; increased microvascular density (MVD, as measured by CD31 IHC) after treatment with IPI-926; increased drug (e.g., gemcitabine) levels in tumor (e.g., post-treatment with either gemcitabine or gemcitabine+IPI-926); and increased uptake of imaging marker Gd-DTPA (DCE-MRI) and fluorescent drug and protein (doxorubicin, lectin) in IPI-926 treated tumors

Assays to measure tumor tissue markers that indicate the effect of hedgehog inhibitors, such as IPI-926, on cancer stroma can be developed. Tumor tissue (e.g., human pancreatic cancer stroma) pre- and post-treatment can be evaluated for the following markers of tumor architecture: Histopathology by H and E, desmoplasia (by evaluating one or more of: collagen, fibronectin, and -SMA), MVD (detection of CD31, Meca32), SPARC (detection by IHC), and reactive stroma signature.

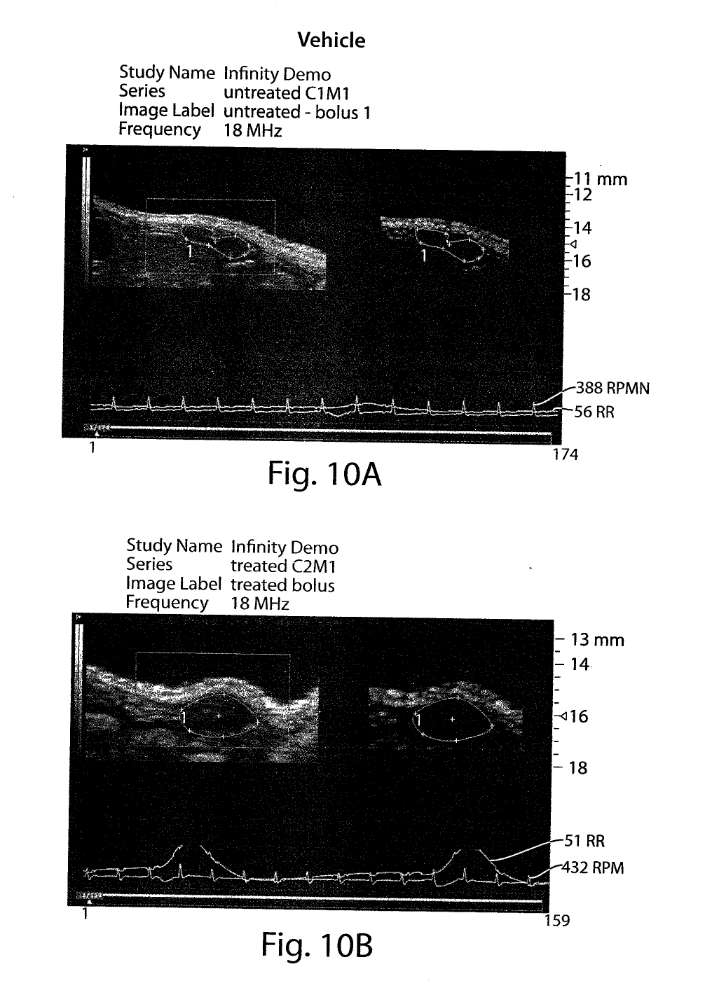

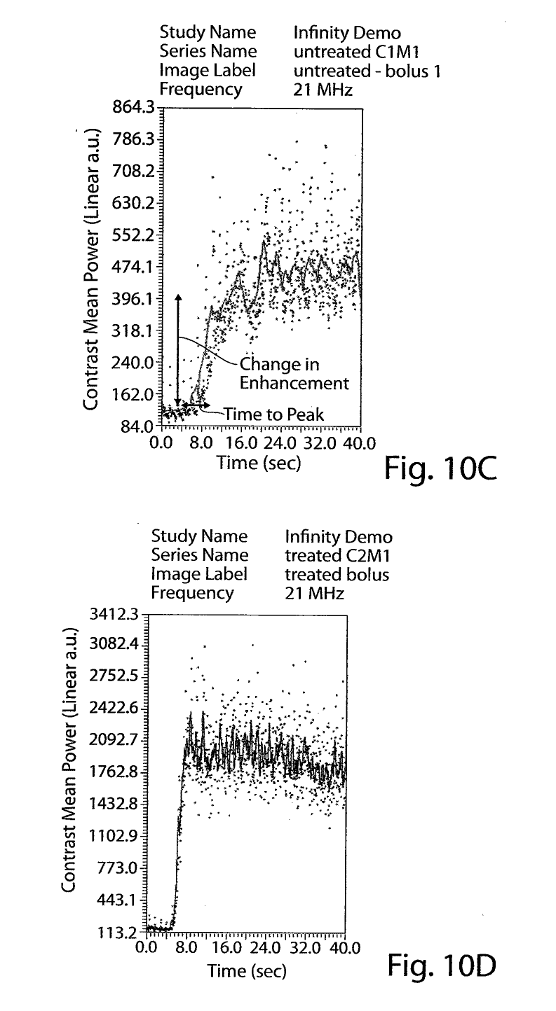

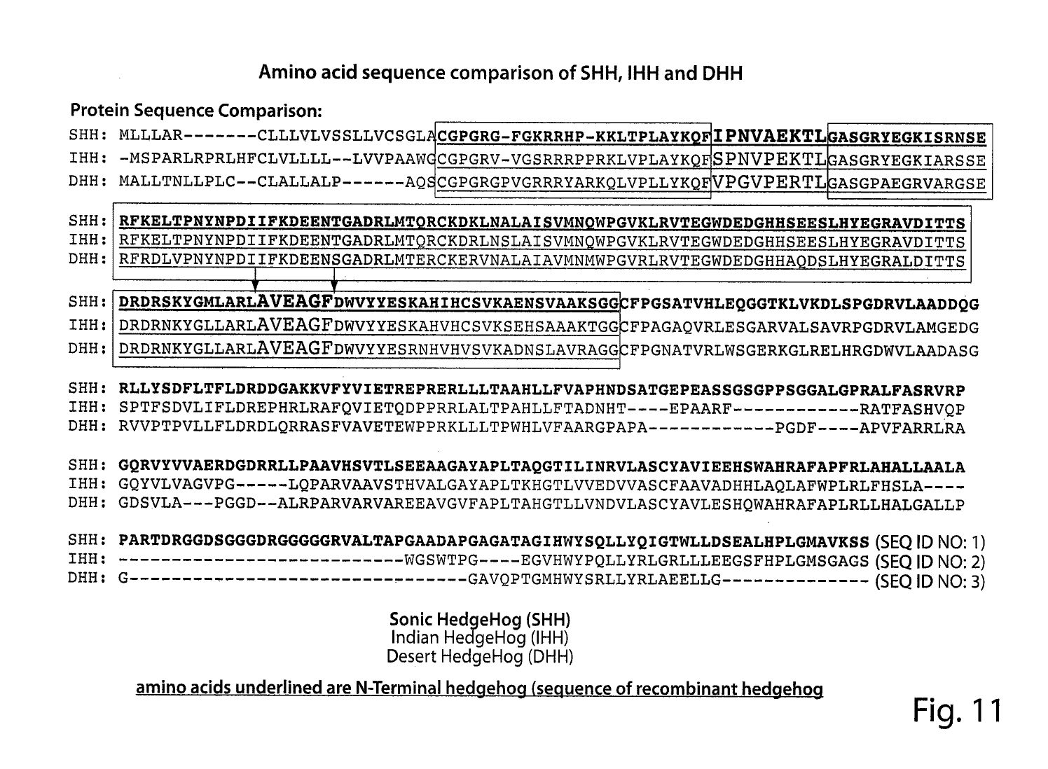

Other assays to measure tumor tissue perfusion pre- and post-IPI-926 treatment can be developed. For example, tumor perfusion can be evaluated by imaging techniques, such as MRI and PET. FIGS. 10A-10B show images from ultrasound measurements of blood perfusion in xenografts. A change in time to peak is detected when comparing untreated with IPI-926-treated samples, FIGS. 10C and 10D, respectively.

### Example 6

### Detection of the Level of Human Sonic, Indian, and Desert Hedgehog in Human Plasma and Sera

This Example describes assays for measuring the level of Human Sonic, Indian, and Desert Hedgehog in Human Plasma and Sera by LC-MS/MS.

In order to quantify the level of hedgehog ligands (SHh, IHh, and DHh) in human plasma and sera by LC-MS/MS, a signature peptide, AVEAGF (SEQ ID NO: 4), has been identified for all these three hedgehog ligands. This peptide sequence can be used to quantify the level of these three Hedgehog proteins. In particular, two assays can be developed: I) Signature Peptide Analytical Method Development and Assay Qualification, and II) Hedgehog (Hh) Protein Quantitation

An alignment of the amino acid sequences of the three ligands is shown in FIG. 5: SHh is shown in the top amino acid sequence; IHh is shown in the middle amino acid sequence; and DHh is shown as the bottom amino acid sequence. The signal peptide sequences for each of the hedgehog ligands are shown in the N-terminal region prior to the highlighted box. Regions of overlap among the three hedgehog ligands are highlighted. The location of the peptide sequence AVEAGF (SEQ ID NO: 4) in common among the hedgehog ligands is indicated by the arrows. The assays can be developed as follows:

### I) Analytical Method Development and Assay Qualification

LC-MS/MS analytical method for quantification of a hedgehog ligand (Hh) by detecting the AVEAGF signature peptide can be developed. A hedgehog ligand in plasma and sera can be digested with proteases. The digested peptides of the hedgehog ligand can be analyzed by LC-MS/MS, mapped, and sequenced against SHh, IHh, and DHh protein primary sequence.

For assay verification, synthetic signature peptide standards can be synthesized and used as calibration curves for quantifying the levels of Hh in human plasma and in sera.

### II) Hedgehog Protein Quantitation

To measure the levels of Hh, the following samples can be obtained:

Study 1: 20 human plasma samples (10 from pancreatic cancer patients and 10 from healthy volunteers),

Study 2: 20 human serum samples (10 from pancreatic cancer patients and 10 from healthy volunteers).

### Reduction and Enzymatic Digestion

The Hh (in plasma and in sera) can be reduced by 2 mM dithiothreitol (“DTT”) and denatured at 100° C. for 15 mM The denatured Hh with all plasma or serum proteins can be digested with a protease. The digested peptides can be analyzed by LC-MS/MS and sequenced against Hh protein sequences.

### Peptide Synthesis

The AVEAGF signature peptide from Hh proteins can be synthesized to confirm LC-MS/MS peptide sequencing (an additional cost for the specific peptide synthesis is included in the budget). The synthetic peptide can be purified by preparative HPLC and analyzed by mass spectrometry to ensure high quality.

### Sample Analysis

The following samples (40 samples in total) can be evaluated:
Study 1: 20 human plasma samples (10 from pancreatic cancer patients and 10 from healthy volunteers),
Study 2: 20 human serum samples (10 from pancreatic cancer patients and 10 from healthy volunteers).

### Example 7

### Identification of IPI-926-Responsive Cells

IPI-926 has been shown to have at least two different effects on tumors growing in experimental animals: 1) Tumor growth inhibition and 2) enhancement of chemotherapeutic drug delivery. Moreover, the former activity is seen under at least three different settings; 1) tumor growth inhibition by IPI-926 as a single agent used in models where tumors are established masses, 2) tumor growth inhibition by IPI-926 where administration begins on the day of, or prior to, tumor cell implant into experimental animals, or 3) tumor growth inhibition by IPI-926 where administration follows a short time after tumor regression with chemotherapy.

Detection of Gli1 mRNA Expression

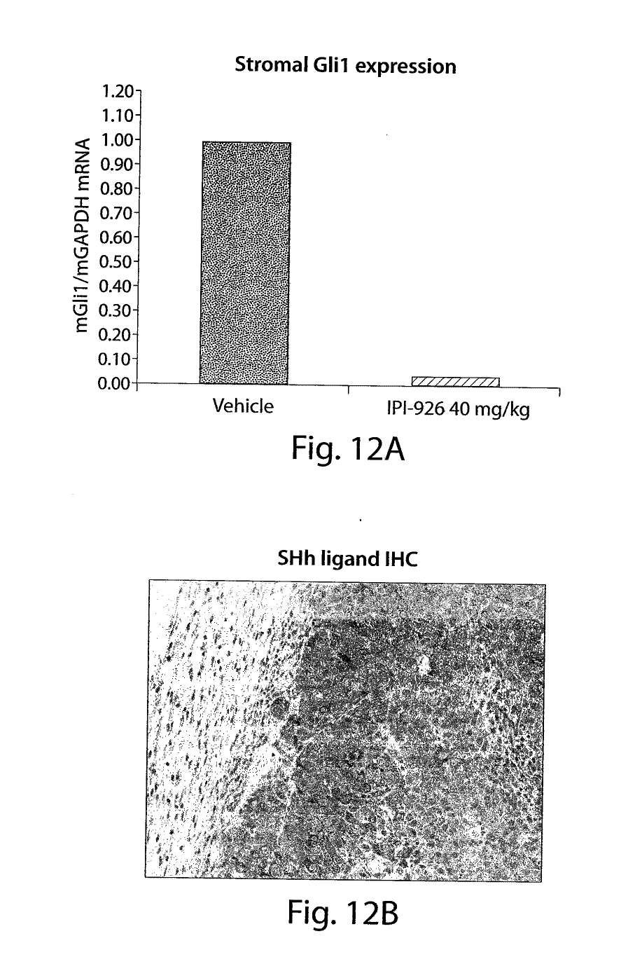

In each of these cases, the action of IPI-926 coincides with downregulation of mRNA for Gli1, an important molecule in the Hedgehog signaling pathway, in the tumor stroma of experimental animals receiving IPI-926 (FIG. 12A). The downregulation of Gli1 mRNA can be used as a functional readout for inhibition of Hedgehog signaling. Moreover, in these models, it appears to be the cancer cells that express the ligand of the Hedgehog signaling pathway (FIG. 12B). Thus, without being bound by theory, Applicants believe that the tumor cells express Hedgehog ligand which signals to a tumor associated stromal cell.

### Detection of Enhanced Growth Inhibition or Chemotherapeutic Drug Delivery/Increased Tumor Perfusion

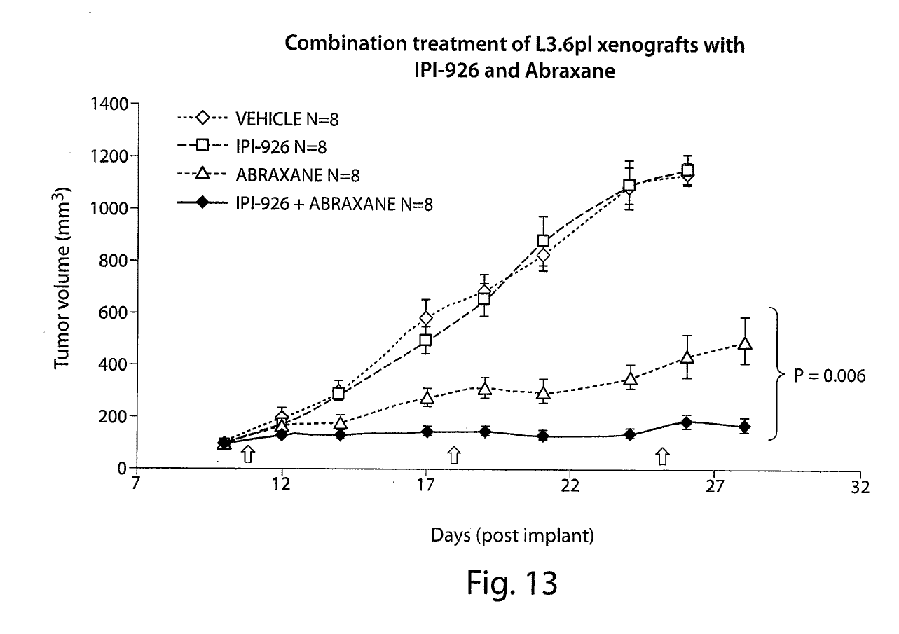

The effect of IPI-926 on this stromal cell then has some effect on the tumor cells, either by growth inhibition, or enhancement of chemotherapeutic drug delivery. For instance, FIG. 13 shows that IPI-926 in combination with Abraxane (albumin-bound paclitaxel) has a synergistic effect on tumor growth inhibition in L3.6pl pancreatic xenograft model. More specifically, FIG. 13 shows the results of increased tumor growth inhibition in the IPI-926 and nab-paclitaxel combination group in the L3.6pl pancreatic xenograft model. The L3.6pl human cell line was implanted subcutaneously and treatment was initiated on Day 10 after implant. IPI-926 was administered orally at 40 mg/kg QOD and nab-paclitaxel was administered i.v. at 20 mg/kg QW1. A) On day 26, compared to the vehicle control, The nab-paclitaxel alone group showed a 61% tumor growth inhibition, while the combination of IPI-926 and nab-paclitaxel resulted in an 83% tumor growth inhibition *(p=0.0048).

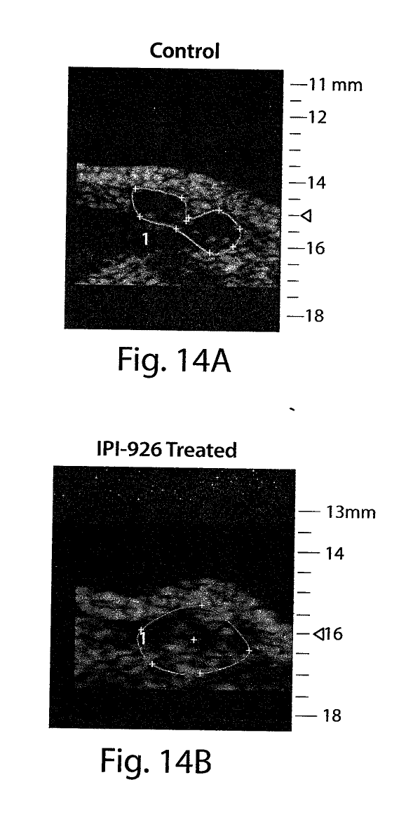

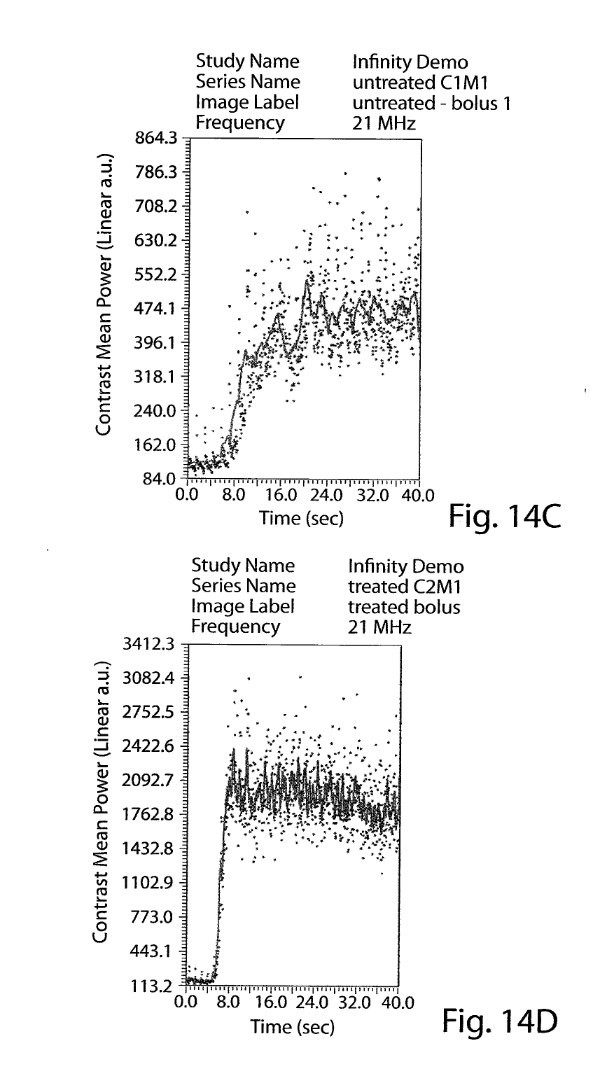

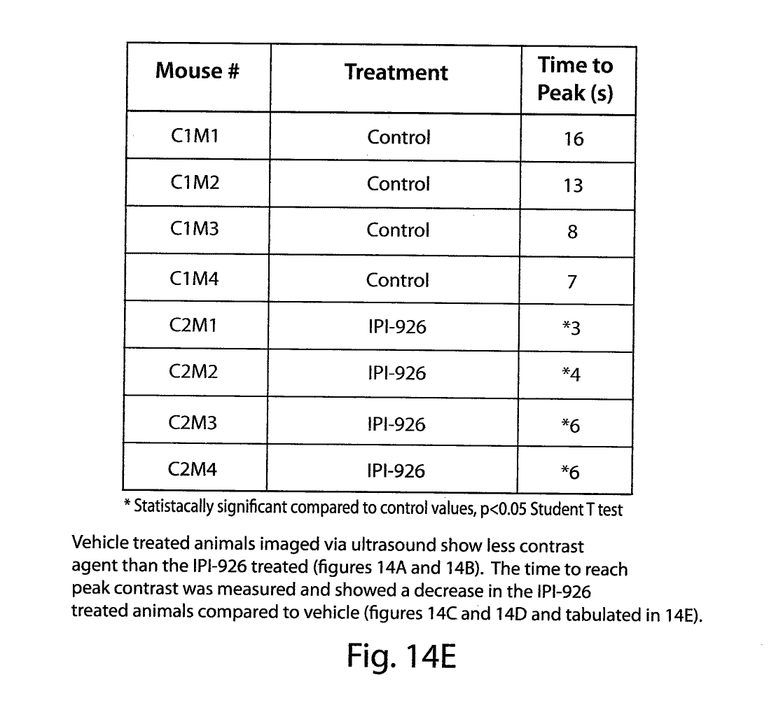

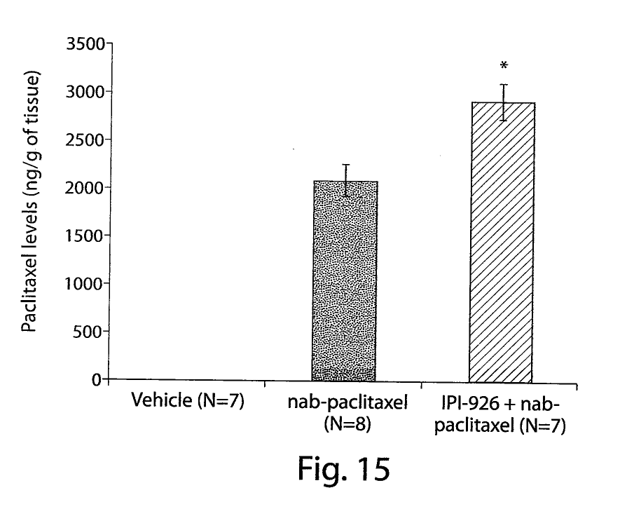

This increase in efficacy of the combination of IPI-926 and Abraxane® was accompanied by an increase in perfusion of tumors in the subcutaneous L3.6pl tumor model, as measured by contrast enhanced high frequency ultrasound (FIGS. 14A-14E). Finally, this was found to also be coincident with higher levels of Abraxane® in the tumor tissue (FIG. 15).

Since tumor-associated stroma is a complex mixture of different cell types, further experiments have been carried out to determine the identity of the IPI-926-responsive cells. Tumors were obtained from animals treated or untreated with IPI-926 and the cells from within the tumors were subsequently fractionated in the following manner. First, the human cancer cells were removed from the mixture using magnetic beads that were coated with an antibody against the cell surface marker, Ep-CAM. Those cells that remained were the murine stromal cells associated with that tumor. Next, these stromal cells were subsequently separated based on their expression of cell surface markers known to be associated with specific cell lineages. In one sample set, cells were separated based on their expression of a panel of markers specific for cells of the hematopoietic lineage. These cells can be referred to as either Lin− or Lin+. In another sample set, cells were separated based on their expression of a cell surface marker specific for endothelial cells, CD31. In either case, like the removal of the human cancer cells, the fractionation of the stromal cells was also achieved through the use of magnetic beads coated with antibodies against lineage specific cell surface markers. Finally, our analysis was completed by measuring Gli1 mRNA expression from the different stromal cell fractions from IPI-926 treated and IPI-926 untreated mice.

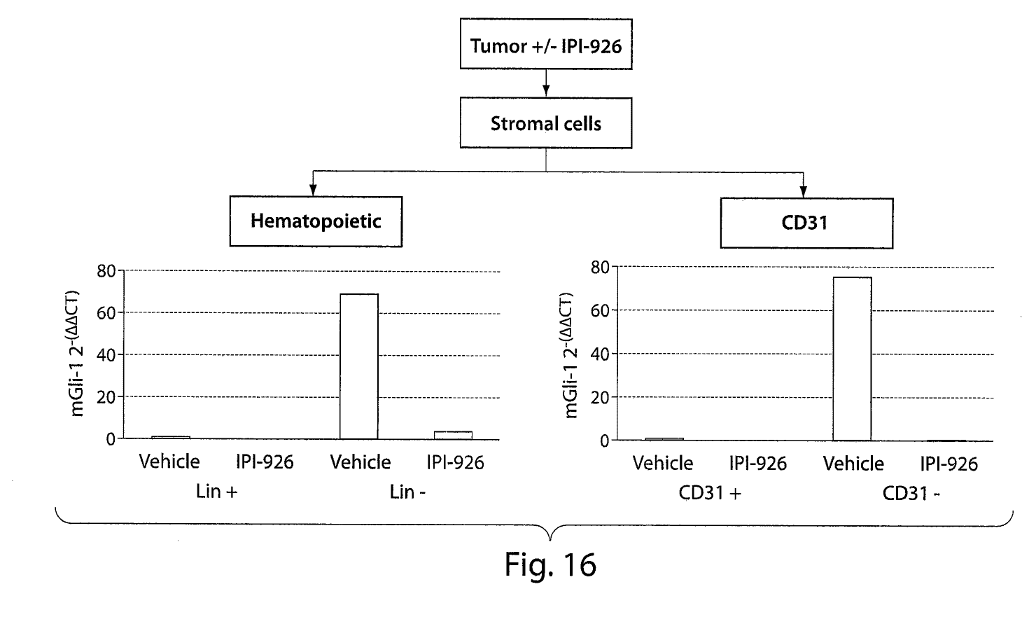

The greatest degree of murine Gli1 inhibition was present in both the Lin− population and CD31− population in samples from IPI-926 treated mice when compared to untreated mice. The IPI-926-responsive stromal cell appears to be a cell from a non-CD31, non-hematopoietic lineage. Thus, the responding cell type appears to be a type of fibroblast within the tumor tissue. Studies are currently ongoing to further refine the identity of the IPI-926 responsive cell. These data are summarized in FIG. 16.

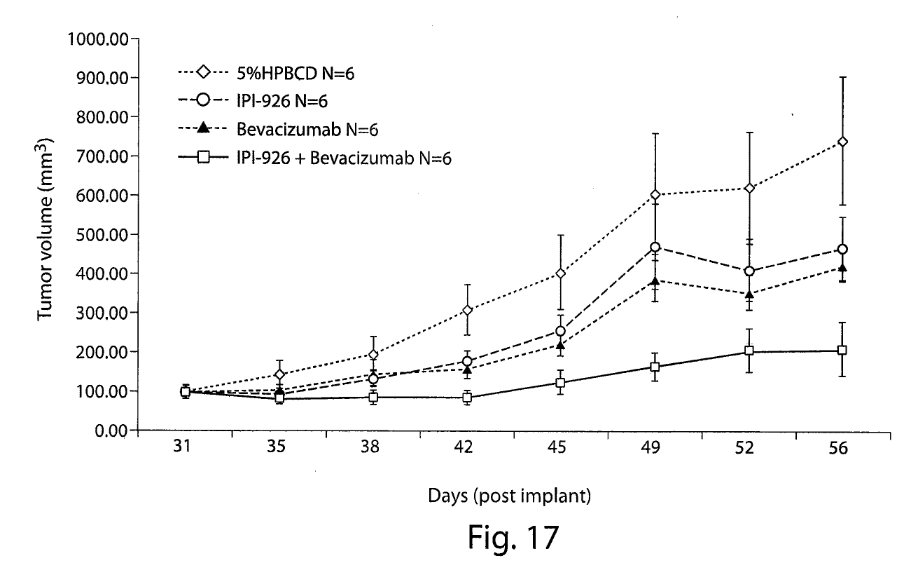

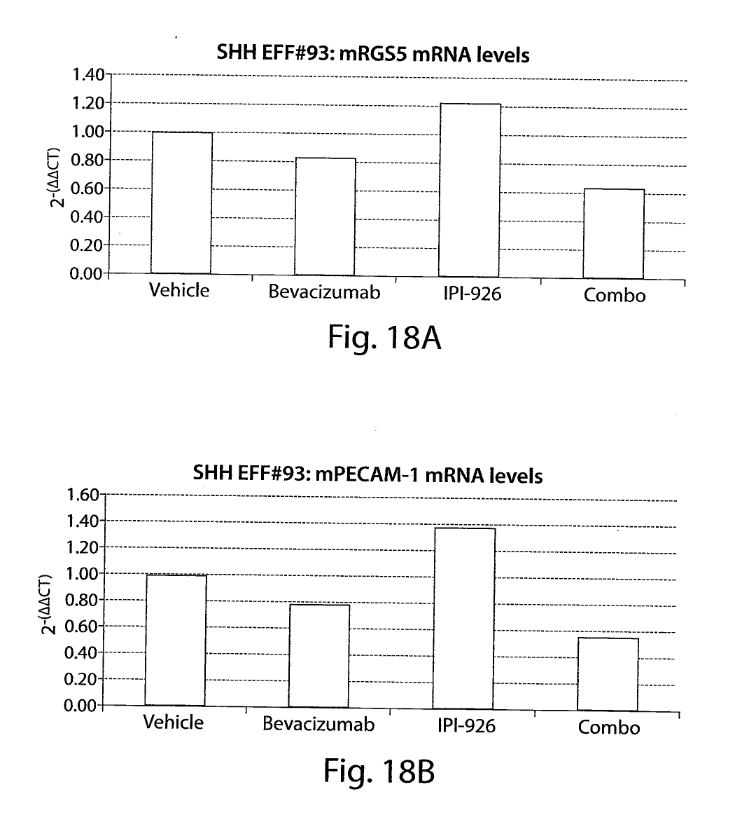

In support of this conclusion, the effect of IPI-926 in combination with Bevacizumab, an agent known to directly affect the endothelial cells of a tumor, was examined. Bevacizumab, a monoclonal antibody, exerts its activity by neutralizing VEGF, a factor important of the proliferation and survival of endothelial cells. In this experiment, both IPI-926 and Bevacizumab had approximately the same single agent activity on the growth of Bx-PC3 tumors in mice. When combined the tumor inhibitory activity appeared to be additive (FIG. 17). In the same study, the relative effect of these agents was explored by measuring the mRNA expression of PECAM-1, a marker of endothelial cells and RGS5, a marker of pericytes. These data show that each agent in each treatment group showed similar effects on both PECAM-1 and RGS5 (FIGS. 18A-18B). The role of the vasculature and its supporting pericyte population in the mechanism of action of IPI-926 continues to be explored.

The aforementioned activity of IPI-926 in enhancing drug delivery to tumors can also lead us to the identity of the IPI-926-responsive cell. A major impediment to the efficacy of current chemotherapeutic agents is the high interstitial fluid pressure in tumors, caused by the poor and disorganized vasculature (Heldin et al (2004) Nature Vol. 4:806). A cell type in the stroma of tumors, the perivascular fibroblast, or pericyte, a specialized type of tissue fibroblast, is involved in the regulation of interstitial fluid pressure. This has been demonstrated using small molecule inhibitors against the cell surface receptor, PDGFR-. which is an important marker on the surface of pericytes Inhibition of PDGFR- has the result of decreasing interstitial fluid pressure with a concomitant increase in chemotherapeutic drug delivery (Pietras et al. (2002) Cancer Research 62:5476-5484); Pietras et al. (2001) Cancer Research 61:2929-2934; Vlahovic et al. (2007) British J. of Cancer 97:735-740). In addition, the inhibition of pericytes with PDGF- inhibitors can also result in inhibition of tumor growth (Bergers et al. (2003) J. of Clin. Invest. 111(9):1287-1295).

Perivascular fibroblasts, or pericytes, are target cells of IPI-926, as the observed activities of IPI-926 are similar in many respects to those activities described for PDGFR-tyrosine kinase inhibitors. Cell sorting experiments are supportive of that contention in that the stromal cell actively signaling in a tumor is a non-hematopoeitic cell and non-CD31 (i.e. endothelial) cell.

IPI-926 enhances chemotherapeutic drug delivery to tumors by influencing the hedgehog signaling pathway in perivascular fibroblasts, i.e., pericytes. This is supported by the observation that inhibition of PDGF signaling in pericytes by agents that target the tyrosine kinase activity of PDGFR- can lead to the enhanced delivery of chemotherapeutics to tumors. This implies that any modulation of pericytes might have this effect and is not likely limited to PDGFR- receptor inhibition directly. For instance it is possible that the ligands of PDGFR-, those being PDGF-B and PDGF-D might mediate enhancement of interstitial fluid pressure of a tumor that expresses these ligands. Therefore, inhibition of PDGF signaling through administration of neutralizing antibodies directed against PDGF-B and/or PDGF-D can cause a decrease in interstitial fluid pressure and a concomitant enhancement of delivery of chemotherapeutic drugs. Moreover, and in more general terms, targeting the pericyte through inhibition of factors or molecular pathways that are important for the pericyte is a viable strategy to enhance the delivery and efficacy of chemotherapeutic agents.

### Methods

### Tumor Xenografting and Tumor Growth

For in vivo studies, tumors were grown in male NCR-nu/nu mice by subcutaneous injection of 5×10⁶ cells. Tumor growth was monitored using calipers. When tumors reached an approximate size of 200 mm³, tumor bearing animals were randomized into treatment arms and administered agents or vehicles as described. For studies designed to monitor tumor growth inhibition, tumor volumes were measured with calipers throughout the study.

### IHC

For immunohistochemical analysis, tissue sections were first de-waxed in changes of xylenes, 100% ethanol and subsequently rehydrated through a graded series of ethanols. Tissue sections were then subjected to 20 minutes of heat-induced antigen retrieval for 20 minutes in citrate buffer. The tissue sections were then blocked for endogenous peroxidase activity using hydrogen peroxide, washed, and then incubated with a primary, anti-sonic hedgehog antibody for 90 minutes at room temperature. After this incubation, the slides were washed 3 times with PBS and then incubated with HRP/polymer-conjugated secondary antibody for 45 minutes. The tissue sections were again washed 3 times with PBS and then incubated with DAB for 5 minutes to develop the staining reaction. Finally, tissue sections were washed, counterstained with hematoxylin and dehydrated for final coverslipping and analysis.

### Cell Sorting

Cells used for sorting experiments were obtained by the following procedure. Tumors from animals that had been administered IPI-926 (40 mg/kg) 24 hours prior to sacrifice were harvested and minced with scalpels. The resulting Brei was subject to enzymatic digestion in a cocktail of collagenase Type II for 2 hours at 37 degrees. After digestion, cells were filtered through a 100 micron mesh and immediately mixed with paramagnetic particles coated with antibodies directed against the human Ep-CAM antigen. After incubation of 30 minutes at four degrees, the beads were passed over a magnetic column and the flow-through collected. The cells remaining on the column were discarded. The cells in the flow-through were then subjected to a similar procedure, but this time using antibodies against the endothelial cell marker CD31, or a cocktail of antibodies directed against the committed cell types of the hematopoietic lineage (i.e. Lin+). These cells were again passed over the magnetic column After this incubation, the cells in the flow through and the column were then analyzed for their expression of specific mRNAs by RT-PCR.

RT-PCR mRNA Analysis

RNA for analysis was isolated from cell pellets or tumor tissue using the Ambion RNA 4-PCR isolation kit according to the manufacturer's guidance. Reverse transcription-PCR was performed for human GAPDH, Gli1 and SHh and murine GAPDH, Gli1, PECAM and RGS5 using an ABI-7300 system with ABI primer/probes. Relative quantification of the expression data was obtained using the ΔΔCT method as described by Applied Biosystems.

### Drug Levels

Levels of Abraxane were estimated by measuring levels of paclitaxel using Liquid chromatography—Mass spectrometry analysis of tumor lysates. Tumor lysates were prepared from frozen samples of tumor tissue. Samples were then injected and then subjected to MS analysis.

### Ultrasound

The L3.6pl tumor cell line was injected subcutaneously and treatment with IPI-926 was initiated. IPI-926 or vehicle was administered orally at 40 mg/kg for seven consecutive days. On the eighth day, animals were subjected to ultrasound image capture and analysis using the Vevo 2100 high frequency ultrasound instrument (Visualsonics) in conjunction with contrast enhancement (microbubbles) during the imaging procedure. Contrast agent was administered by iv administration during the imaging procedure and quantified by measuring echogenic signal derived from the contrast agent over time.

### Example 8

### Genes Differentially Expressed in Chondrosarcoma

Biomarkers differentially expressed in chondrosarcoma were identified by Affymetrix gene expression profiling.

After hybridization and detection, raw data from one color Affymetrix experiment was read into the R/BioConductor environment with control spots removed. An object of type “ExpressionSet” was created from this data and associated experiment details. The “vsn” library was used to perform Variance Stabilization Normalization via the “justvsn” function. Data was fit to a linear model using “lmfit” from the “limma” library and standard errors estimated using empirical Bayes moderation using “lmFit” (also from “limma”). Probes with an adjusted P-value <=0.05 were selected (using “topTable” from “limma” to yield the final list, which was annotated for gene symbols using the “hgug4112a.db” and “annotate” libraries. All named libraries are standard BioConductor libraries.

Table 1 shows a list of exemplary biomarkers differentially expressed in chondrosarcoma. Table 2 shows the fold change for the exemplary biomarkers listed in Table 1.

TABLE 2

| GenBank ™ | Sequence | Probe Set/ | | Fold |
|---|---|---|---|---|
| Accession No. | ID | Sequence Code | Gene | Change |
| NM_001040272 | 1485850 | A_23_P342138 | ADAMTSL1 | −3.55 |
| NM_032515 | 1471770 | A_23_P253029 | BOK | −3.56 |
| NM_000587 | 1466974 | A_23_P213857 | C7 | −3.71 |
| NM_001266 | 1482536 | A_23_P206733 | CES1 | −4.35 |
| NM_033181 | 1483224 | A_23_P214208 | CNR1 | −5.53 |
| NM_007207 | 1479296 | A_24_P182494 | DUSP10 | −3.03 |
| NM_001002919 | 1497721 | A_24_P561341 | FAM150B | −4.42 |
| AK095698 | 1472689 | A_23_P313031 | FLJ38379 | −3.43 |
| BG216229 | 1495860 | A_23_P135123 | FRMD3 | −3.36 |
| NM_004962 | 1491440 | A_24_P355464 | GDF10 | −4.91 |
| NM_005269 | 1472019 | A_23_P105251 | GLI1 | −6.52 |
| NM_001010931 | 1463981 | A_23_P93780 | HGF | −3.30 |
| NM_022475 | 1473036 | A_23_P167129 | HhIP | −5.27 |
| BC007638 | 1465040 | A_23_P86059 | HhIPL2 | −3.84 |
| NM_000212 | 1480360 | A_24_P318656 | ITGB3 | −3.61 |
| NM_014592 | 1496354 | A_23_P30554 | KCNIP1 | −5.29 |
| NM_005559 | 1478652 | A_24_P100613 | LAMA1 | −2.93 |
| NR_001443 | 1501447 | A_32_P46238 | LOC339240 | −3.88 |
| NM_032445 | 1489033 | A_23_P54340 | MEGF11 | −4.14 |
| NM_001005473 | 1485632 | A_32_P119033 | PLCXD3 | −5.66 |
| NM_006744 | 1483670 | A_23_P75283 | RBP4 | −3.86 |
| NM_006142 | 1490730 | A_23_P63254 | SFN | −3.06 |
| NM_012309 | 1485772 | A_23_P334883 | SHANK2 | −3.09 |
| NM_177550 | 1491165 | A_23_P66739 | SLC13A5 | −5.38 |
| NM_007191 | 1479354 | A_32_P216520 | WIF1 | 4.18 |
| NM_003862 | 1498008 | A_23_P412389 | FGF18 | 3.79 |
| NM_006398 | 1473246 | A_23_P81898 | UBD | 3.41 |
| NM_021146 | 1462986 | A_23_P114862 | ANGPTL7 | 3.34 |
| NM_001042 | 1476242 | A_32_P151263 | SLC2A4 | 3.14 |

Thus, changes in the level or activity of one or more of the biomarkers described in Table 1 or 2 can be used to evaluate disease progression and/or responsiveness to Hh inhibition.

### Example 9

### Detection of Nuclear Gli1 Staining in Tumor Samples

Tumor and stromal nuclear Gli1 staining can be detected in samples obtained from cancer patients by methods known in the art for nucleic acid or protein detection. This example describes protocols for immunohistochemical (IHC) detection of Gli1 protein. However, any method available in the art for Gli1 nucleic acid or protein detection can be used.

For immunohistochemical (IHC) detection, tissue samples can be fixed in 10% neutral buffered formalin at room temperature before processing for paraffin embedding. The length of fixation depends on the size of the tissue, for example, overnight but not more than 22-24 hours for large tissues; and no more than 2 hours for small core or needle biopsies. The fixed tissues are sectioned to 7 microns (mm) and mounted on Superfrost Microscope™ slides (Fisher Scientific).

Prior to incubation with anti-Gli1 antibody, antigen retrieval can be performed to break the protein cross-links formed by formalin fixation and thereby reveal hidden antigens. Antigen retrieval buffers with different buffer compositions and pH are commercially available, for example, CC1 (Tris/EDTA, pH 8.0; Ventana, Cat. No. 950-124); CC2 (citrate, pH 6.0; Ventana, Cat. No. 950-123); citrate buffer (pH 6.0; Invitrogen, Cat. No. 00-5000); Target Retrieval Solution (citrate, pH 6.1; Dako, Cat. No. S170084); Target Retrieval Solution (Tris/EDTA, pH 9.0; Dako, Cat. No. S236884); Diva Decloaker (citrate, pH 6.2; Biocare, Cat. No. DV2004 MM); and EDTA Decloaker (EDTA based, pH 8.5; Biocare, Cat. No. CB917 L).

Reagents for blocking non-specific binding of primary and/or secondary antibodies can also be used. For example, blocking reagents such as high ionic strength protein reagents can be dispensed into the same buffer pool as primary and/or secondary antibodies and incubated together. Commercially available blocking reagents for immunohistochemistry include, e.g., Discovery Antibody Block™ (Ventana, Cat. No. 760-4204); Enhanced Blocking Reagents for IHC (General Bioscience, Cat. No. SU-002-110); Hydrogen Peroxide Blocking Reagent (Abcam, Cat. No. ab94666); IHC Select® HRP Detection Set (Millipore, Cat. No. DET-HP1000); Protein Blocking Reagent (animal serum free) (GeneTex, Cat. No. GTX30963); and Ready-To-Use IHC Blocking Reagent (Bethyl Laboratories, Inc., Cat. No. IHC-101B).

Anti-Gli1 antibodies that can be used to detect nuclear Gli1 protein in tumor samples are commercially available. Exemplary anti-Gli1 antibodies include, e.g., rabbit polyclonal and monoclonal antibody from Cell Signaling Technology (Cat. Nos. 2553 and 3538, respectively); rabbit polyclonal antibody (H-300) from Santa Cruz Biotechnology (Cat. No. sc-20687, described in Chung et al., J. Clin. Oncol. 2011; 29(10):1326-34); rabbit polyclonal antibody from Abcam (Cat. No. ab92611); rabbit polyclonal antibody from Abnova (Cat. No. PAB 10214); rabbit polyclonal antibody from GenWay Biotech (Cat. No. 18-732-292074); rabbit polyclonal antibody from LifeSpan BioSciences (Cat. No. LS-C66529-50); rabbit polyclonal antibody from Novus Biologicals (Cat. No. NBP1-03294); mouse polyclonal antibody from ProSci (Cat. No. 49-519); goat polyclonal antibody from R&D Systems (Cat. No. AF3455); and rabbit polyclonal antibody from United States Biological (Cat. No. G2035-51A).

Detection of anti-Gli1 antibodies in tumor samples can be enhanced using amplification reagents. Exemplary commercially available immunohistochemistry amplification reagents include, but not limited to, Amplification Kit (Ventana, Cat. No. 760-080); Tyramide Signal Amplification (TSA™) Kit (Invitrogen, Cat. No. T20921); Goat anti-Rabbit IgG, Poly-HRP, Ampli-HRP (Millipore, Cat. No. AP342P-50mL); Goat anti-Mouse IgG, Poly-HRP, Ampli-HRP (Millipore, Cat. No. AP340P-50mL); Super Amplifying IHC Kit with Antibody Amplifier™ (IHC WORLD, Cat. No. IW-AAk-4); and Super Amplifying IHC Kit with Antibody Amplifier Eclipse™ (IHC WORLD, Cat. No. IW-AAk-4).

Antibody detection can be performed using commercially available immunohistochemical detection reagents, including, e.g., DABMap™ Kit (Ventana, Cat. No. 760-124); IHC Select® HRP Detection Set (Millipore, Cat. No. DET-HP1000); Peroxidase IHC Detection Kit (Pierce, Cat. No. 36000); SignalStain® Boost IHC Detection Reagent (HRP, Rabbit) (Cell Signaling Technology, Cat. Nos. 8114 (HRP, Rabbit) and 8125 (HRP, Mouse); and TruVision™ Poly-HRP IHC Detection Kit (anti-mouse/rabbit with DAB) (General Bioscience Corporation, Cat. No. IHC-701).

Platforms for immunohistochemical staining are commercially available. For example, nuclear Gli1 staining can be performed on platforms or instruments, such as Discovery® XT System (Ventana, Tucson, Ariz., USA); and Autostainer Link 48 (Dako, Cat. No. AS480).

### Example 10

### Detection of Nuclear Gli1 Staining in Tumor and Stroma of Tumor Samples

Detection of tumor nuclear Gli1 staining in basal cell carcinomas (BCC) was performed using the protocols described in Example 9. FIGS. 23C and 23D show nuclear staining of Gli1 in biopsy samples of a BCC lesion pre- and 22-day post-treatment of IPI-926.

Elevated Gli1 staining in the nucleus of tumor cells (in terms of expression levels and increased number of GLI1-positive tumor cells) are found in tumor samples showing advanced disease (FIG. 23C). A reduction in terms of the Gli1 expression and number of Gli1-positive cells is detected after treatment with IPI-926 (FIG. 23D). Thus, elevated Gli1 nuclear staining in BCC tumor samples is indicative of the severity of the disease, and predictive of responsiveness to hedgehog inhibition. Images of the BCC lesions in a patient before and 6-month after IPI-926 treatment are also shown in FIGS. 23A-23B, respectively. A dramatic reduction in the size and morphology of the lesion is detected in the patient 6-month after treatment.

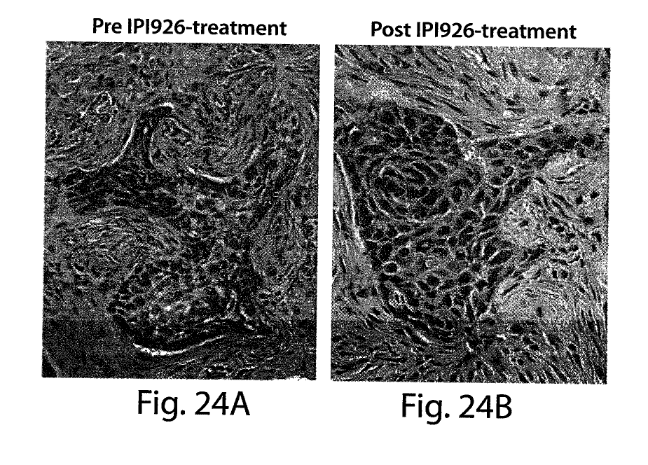

Hematoxylin and eosin (H&E) staining showing nuclear and cytoplasmic staining in biopsy samples of a BCC lesion pre- and post-IPI-926 treatment are depicted in FIGS. 24A-24B. Methods for H&E staining are described in, e.g., Lillie R. D. et al., (1976) Histochemistry 49: 23-35. These results indicate unusual clearing of nuclei in Gli1 immunostained tumors after IPI-926 treatment, but similar nuclear changes were not observed on H&E staining.

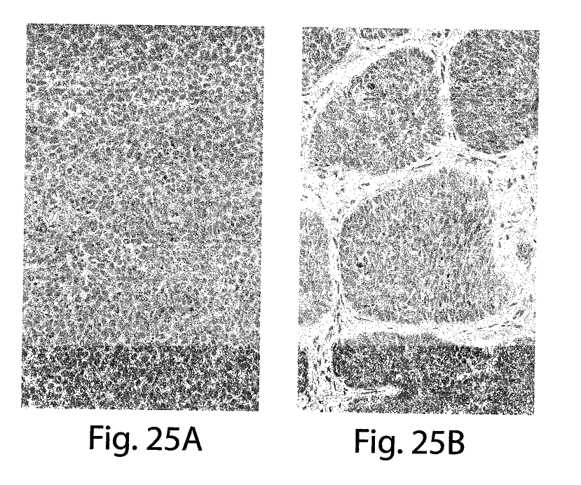

Nuclear staining for Gli1 is noted in both pre and post treatment samples with the post treatment sample showing a 60% reduction in nuclear Gli1 stain and an overall reduction of Gli1 protein staining by 57%. In the same BCC samples, 27% of the nuclei stained positive for Gli1 (H-score=100) before IPI-926 treatment, compared to 16% (H-score=57) post-treatment (FIGS. 25A-25B).

The definition of H-score is known in the art, e.g., as described in Detre, S. et al. (1995) J. Clin. Pathol. 48:876-878. H-scores are between 0 and 300. Briefly, it refers to the intensity of staining (rated from 0-3) times the percentage of tumor cells expressing the gene of interest. Typically for H-score assessment, several fields (e.g., 10) are chosen at random at, e.g., a 400× magnification. The intensity in the malignant cell nuclei can be scored as 0, 1, 2, or 3. Score 3 refers to strong and consistent staining pattern on nuclei across the entire cross-section of the nucleus; Score 2 refers to nucleus visible with staining and no evidence of any differential staining of the nuclear envelop; Score 1 refers to evidence of differential staining of the nuclear envelop but staining across the nucleus still present; and Score 0 refers to clear and strong ring-like staining pattern with minimal or no staining across the nucleus. The total number of cells in each field and the number of cells stained at each intensity are counted. The average percentage positive can be calculated and analyzed as described in Detre, S. et al. (1995) supra.

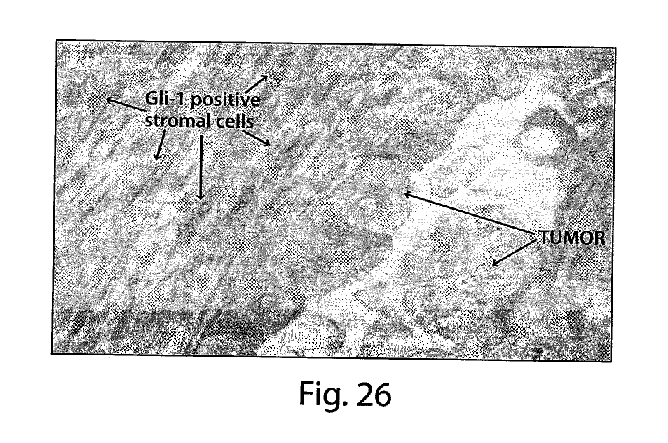

Detection of stromal nuclear Gli1 staining in pancreatic cancer was performed using the protocols described in Example 9. As shown in FIG. 26, nuclear Gli1 staining was observed in the stromal cells. Gli1 expression appears to be predominantly found in stromal cells, but not in pancreatic tumor cells which is consistent with a paracrine signaling mechanism in the stromal cells of pancreatic tumors. The presence of active paracrine signaling in the stromal cells of the pancreatic tumor samples indicates a therapeutic role for IPI-926.

Similar results regarding tumor and stromal nuclear Gli1 staining were observed in some of the other tumor types. A summary of cases stained for Gli1 by immunohistochemistry is shown in Table 3.

TABLE 3

| Cases Stained | Positive | Negative |
|---|---|---|
| n = 49 BCCs | 43 (88%) | 6 (12%) |
| n = 80 cutaneous SCCs | 0 (0%) | 80 (100%) |
| n = 29 Head & Neck SCCs | 0 (0%) | 29 (100%) |
| n = 7 metastatic | 3 (43%) | 4 (57%) |
| adenocarcinomas | *staining in the stroma | |
| n = 5 chondrosarcomas | 3 (60%) | 2 (40%) |

In Table 3, BCC and SCC refer to basal cell carcinoma and squamous-cell carcinoma, respectively. Nuclear localization of Gli1 was detected in chondrosarcoma samples (data not shown).

### Example 11

### Comparative GLI1 mRNA Detection

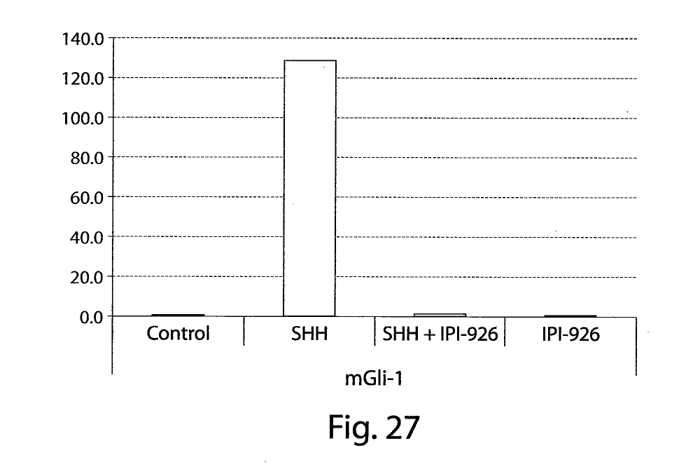

As a comparison, real-time RT-PCR was performed using RNA extracted from Laser Capture Microdissected tumor samples from BCC patients. The ddCt method was used to calculate the real-time PCR results (Livak K. J. et al. (2001) Methods. 25(4):402-8). FIG. 27 is a bar graph depicting the quantitation of Gli1 mRNA pre- and post treatment with IPI-926. Although a decrease level of Gli1 mRNA is detected post-treatment, the level of reduction is not as pronounced as that detected by evaluating nuclear localization of Gli1.

Real-time RT-PCR was also performed using RNA extracted from Laser Capture Microdissected tumor and stromal samples from pancreatic cancer patients to measure the expression of Gli1. Briefly, RNA was isolated using Qiagen RNeasy FFPE kit (Cat. No. 73504), and real-time RT-PCR was performed on ABI 7900HT using Taqman® probes and reagents obtained from Applied Biosystem (Gli1: Cat. No. Hs00171790_m1; GADPH: Cat. No. 4310884E-0904043). Consistent with the immunohistochemistry data, the real-time RT-PCR showed higher expression of Gli1 in stroma relative to tumor of each sample.

### Example 12

### RGS5 as a Target of SHh and IPI-926

In order to identify a cell target of IPI-926 and its mechanism of action, perivascular fibroblasts, or pericytes, were evaluated as a stromal cell target of SHh signaling from the tumor.

To test this, a model of pericyte cell differentiation, the C3H10T1/2 cells was evaluated. C3H10T1/2 cells were differentiated in vitro into pericytes. C3H10T1/2 cells are believed to be pluripotent mesenchymal cells, which can be induced to differentiate into multiple different cell lineages, including myoblasts, adipocytes, osteoblasts and pericytes (Taylor, et al. (1979) Cell 17(4):771-9). In this case, cells are cultured to confluence and then allowed to differentiate over the course of 5 additional days of culture. At this time, the cells are seen to express high levels of CD13, an established marker of pericytes.

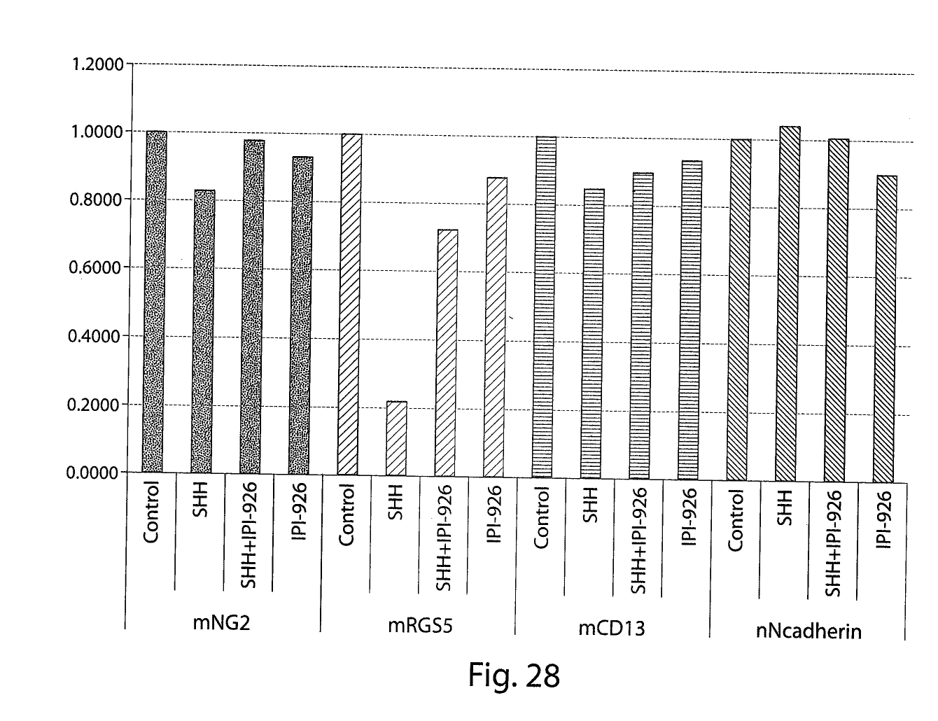

We subsequently explored if SHh signaling was intact in these pericyte cultures. In a typical experiment, confluent C3H10T1/2 cells were allowed to differentiate into pericyte-like cells. After 5 days in culture, SHh with or without IPI-926 were added to the cells in the presence of normal serum. On day 8 of culture, cells were re-fed SHh with or without IPI-926 and shifter to low serum. qRT-PCR analysis was performed on day 9. SHh signaling was detected in these cells as measured by Gli1 mRNA upregulation; the SHh signaling was inhibited by IPI-926 (FIG. 28). Cells were treated on day 8; Gli1 mRNA levels were evaluated in control samples, after SHh addition, after SHh and IPI-926 and cells treated with IPI-926 alone as shown in FIG. 28. Cells were collected 72 hours after treatment. Of particular interest was the observation that the expression of RGS5 mRNA appears to be downregulated by SHh, and this downregulation is reversed by IPI-926 in these pericyte cultures (FIG. 29).

The RGS5 gene is of particular interest for several reasons. First, RGS5 is a well validated marker of the pericyte lineage of cells, thus supporting the view that the pericyte is indeed a target cell of IPI-926 action. Moreover, the modulation of RGS5 by IPI-926 suggests that one action of SHh and thus, IPI-926 might be to modulate pericyte responsiveness to other signals, rather than IPI-926 having a direct effect on pericyte function. This view comes from what is known about RGS5 and RGS proteins in general. The RGS family of proteins are molecules that modulate, or fine-tune, signaling through GPCR molecules. They do so by inhibiting signaling flux through GPCR molecules (Cho, H. et al. (2003) FASEB J 17(3):440-2). Therefore, as SHh downregulates RGS5 expression, this would have the effect of increasing signaling through its cognate GPCR. In contrast, re-establishment of RGS5 expression levels in the presence of IPI-926 would, therefore, act to inhibit signal flux through its cognate GPCR. It is, therefore, possible that SHh and IPI-926 modulate other signals in the tumor stromal microenvironment and that these co-signaling molecules can prove to be useful predictive biomarkers for the action of IPI-926. For instance, Sphingosine-1-phosphate, the ligand for S1PR1 (a GPCR) and PDGF-BB have been reported to be important molecules in the growth and function of pericytes. Both of these molecules have also been demonstrated to be influenced by RGS5 (Cho, H. et al. (2003) supra). Therefore, the presence of one or both of these molecules in a tumor can be used to predict whether IPI-926 has a modulatory effect on the signal transduction initiated by these molecules. In other words, the presence of PDGF-BB and or S1P suggests that signaling through these axes might be active, and therefore susceptible to inhibition by IPI-926. If on the other hand these molecules were not present, then signaling through these pathways would not be expected to be active, and as such, IPI-926 would be predicted to not have a modulatory effect.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for evaluating a cancer or tumor sample, comprising detecting a hedgehog-associated biomarker chosen from one or more of: nuclear Gli1, the presence or absence of a cilium marker, an alteration in desmoplasia, or an alteration in a pericytic marker, in the sample, wherein one or more of: an increase in nuclear Gli1, the presence of the cilium marker, an alteration in desmoplasia, or an increased level of the pericytic marker, indicates an increased likelihood of responsiveness of the cancer or tumor to a hedgehog inhibitor.

2. The method of claim 1, further comprising one or more of the following:
(i) identifying a subject having a cancer or a tumor, or at risk of developing a cancer or a tumor, as having an increased or a decreased likelihood to respond to treatment with a hedgehog inhibitor;
(ii) selecting or altering one or more of the course of therapy, dosing, treatment schedule, or combination therapy;
(iii) analyzing a time course of the cancer or tumor in the subject; or
(iv) analyzing the probability of a significant event in the subject with the cancer or tumor.

3. The method of claim 1, further comprising comparing the level of the hedgehog-associated biomarker to a reference value or sample.

4. A method for identifying a subject having a cancer or a tumor, or at risk for developing a cancer or a tumor, as having an increased or decreased likelihood to respond to a treatment with a hedgehog inhibitor, comprising:
evaluating a sample from the subject to detect a hedgehog-associated biomarker chosen from one or more of: nuclear Gli1, the presence or absence of primary cilia or a phosphorylated hegdehog receptor in the cilia, an alteration in desmoplasia, or an alteration in a pericytic marker; and
identifying the subject having the cancer or at risk for developing the cancer as likely or unlikely to respond to the treatment with the hedgehog inhibitor, wherein one or more of: an increase in nuclear Gli1, the presence of the primary cilia or a phosphorylated hegdehog receptor in the cilia, an alteration in desmoplasia, or an increased level of the pericytic marker, indicates that the subject has an increased likelihood to respond to treatment with the hedgehog inhibitor.

5. A method for evaluating or monitoring a cancer therapy treatment regimen in a subject having a cancer or tumor, or at risk for developing cancer or tumor, comprising:
evaluating a sample from the subject to detect a hedgehog-associated biomarker chosen from one or more of: nuclear Gli1, the presence or absence of primary cilia, an alteration in desmoplasia, or an alteration in a pericytic marker; and
selecting or altering one or more of the course of therapy, dosing, treatment schedule, or a combination therapy,
wherein one or more of: a decrease in the nuclear Gli1, the presence of the primary cilia or the phosphorylated hegdehog receptor in the cilia, an alteration in desmoplasia, or level of the pericytic marker, is indicative of improved therapeutic outcome.

6. A method for evaluating a time course of a cancer, and/or the probability of a significant event, in a subject having a cancer or a tumor, or at risk for developing a cancer or a tumor, comprising:
evaluating a sample from the subject to detect a hedgehog-associated biomarker chosen from one or more of: nuclear Gli1, the presence or absence of primary cilia or a phosphorylated hegdehog receptor in the cilia, an alteration in desmoplasia, or an alteration in a pericytic marker; and
comparing the detected alteration to a reference value or sample,
wherein one or more of: a decrease in nuclear Gli1, the presence of the primary cilia or the phosphorylated hegdehog receptor in the cilia, an alteration in desmoplasia, or level of the pericytic marker, is indicative of improved prognosis.

7. The method of any of claim 1 or 4-6, further comprising treating the subject with a hedgehog inhibitor, alone or in combination with one or more of another chemotherapeutic agent, surgery and/or radiation.

8. The method of claim 1, wherein the cilium marker is a primary cilium or a component thereof chosen from one or more of: a microtubule or a component thereof, tubulin, a component of intraflagellar transport (IFT), a kinesin, a microtubule organizing center or a component thereof, a basal body or a component thereof, or a phosphorylated hedgehog receptor.

9. The method of claim 1, wherein the hedgehog biomarker evaluated further comprises evaluating one or more alterations in a marker of a hedgehog pathway, an alteration in a genomic marker, an alteration in a marker of Epithelial to Mesenchymal Transition (EMT), an alteration in a Gemcitabine marker, or an alteration in tumor architecture.

10. The method of claim 1, wherein the hedgehog biomarker evaluated further comprises evaluating one or more of:
(i) an alteration in a gene or a gene product of a hedgehog ligand chosen from Sonic Hedgehog (SHh), Indian Hedgehog (IHh) or Desert Hedgehog (DHh));
(ii) an alteration in a gene or a gene product of chosen from one or more of SMO or PTCH, SUFU, GLI3, or BOC;
(iii) an alteration in a gene or a gene product chosen from KRAS, TGFβ-SMADs, p53, cyclin D1, ALK, EGFR, PIK3CA, BRAF, PTEN, AKT, TP53, NRAS, CTNNB1 (beta-catenin), APC, K1T, JAK2, NOTCH, or FLT3;
(iv) an alteration in a marker of Epithelial to Mesenchymal Transition (EMT), chosen from one or more of snail, twist, slug, vimentin, cadherins, or SPARC;
(v) an alteration in a Gemcitabine markers chosen from SLC29A1, SLC28A1, SLC28A3, CDA, NT5C, DCK, UMP/CMP kinase, RRM1, RRM2, Nucleoside diphosphate kinase, or HuR; or
(vii) an alteration in tumor architecture chosen from an alteration in tumor size; an alteration in collagen, fibronectin, or alpha-smooth muscle-specific actin (SMA) levels; an alteration in tumor perfusion; an alteration in interstitial fluid pressure; an alteration in microvascular density (MVD); an alteration in a marker chosen from CD31 or Meca32; an alteration in pericytes; or an alteration in markers chosen from NG-2 (CSPG4), RGS5, N-cadherin, PDGFR-beta.

11. The method of claim 1, wherein the tumor or cancer sample is from a chondrosarcoma and the evaluation further comprises one or more of: a histological evaluation; cytogenetic analysis; changes in cytogenic molecular markers, such as LOH at loci; evaluation of osteogenic lesions by Magnetic Resonance Imaging (MRI) or CT-scan; or detection of one or more of type II collagen, MIB-1, p53, or Ki-MCM6.

12. The method of claim 1, wherein the tumor or cancer sample is from a chondrosarcoma and the evaluation further comprises detection of the level of expression of one or more of: ADAMTSL1, BOK, C7, CES1, CNR1, DUSP10, FAM150B, F1138379, FRMD3, GDF10, Gli1, HGF, HhIP, ITGB3, KCNIP1, LAMA1, LOC339240, MEGF11, PLCXD3, RBP4, SFN, SHANK2, WIF1, FGF18, UBD, ANGPTL7 or SLC2A4.

13. A method of treating a cancer or tumor harboring a hedgehog-associated biomarker chosen from one or more of: an increased nuclear Gli1, the presence of a cilium marker, an alteration in desmoplasia, or level of a pericytic marker, comprising administering to a subject a hedgehog inhibitor, alone or in combination with another therapeutic agent or radiation, in an amount sufficient to treat the cancer, in the subject, wherein the subject has been previously evaluated for the one or more hedgehog-associated markers.

14. A method of enhancing delivery of a therapeutic agent to a tumor or a cancer cell, comprising:
contacting a tumor or a cancer cell in a subject with, or administering to a subject, a hedgehog inhibitor, alone or in combination with the therapeutic agent, in an amount sufficient to increase the delivery of the therapeutic agent to the tumor or a cancer cell, wherein the subject has been previously evaluated for the presence a hedgehog-associated biomarker chosen from one or more of: an increased nuclear Gli1, the presence of a cilium marker, an alteration in desmoplasia, or level of a pericytic marker

15. The method of claim 13, wherein the subject identified or treated is a human having, or at risk of having, the cancer or tumor, wherein the cancer or tumor is hedgehog-independent or a hedgehog dependent cancer.

16. The method of claim 15, wherein the cancer or tumor is chosen from one or more of: bladder cancer, breast cancer, medulloblastoma, colorectal cancer, head and neck cancer, lung cancer, acute lymphoblastic leukemia (ALL), acute myeloid leukemia (AML), chronic myelogeneous leukemia (CML), chronic lymphocytic leukemia (CLL)), lymphoma (e.g., Hodgkin lymphoma (HL), non-Hodgkin lymphoma (NHL)), multiple myeloma (MM), chronic myeloproliferative disorder, primary myelofibrosis, polycythemia vera, essential thrombocytemia, osteosarcoma, ovarian cancer, pancreatic cancer, prostate cancer, basal cell carcinoma (BCC)) or chondrosarcoma.

17. The method of claim 16, further comprising the step of monitoring the subject for a change in one or more of: tumor size; stromal activation; levels of one or more cancer markers; the rate of appearance of new lesions; the appearance of new disease-related symptoms; the size of soft tissue mass; quality of life; amount of disease-associated pain.

18. The method of claim 16, further comprising monitoring the subject in one or more of the following periods: prior to beginning of treatment; during the treatment; or after one or more elements of the treatment have been administered.

19. The method of any of claim 1-6, or 13-14, wherein the hedgehog inhibitor is a compound of the formula: 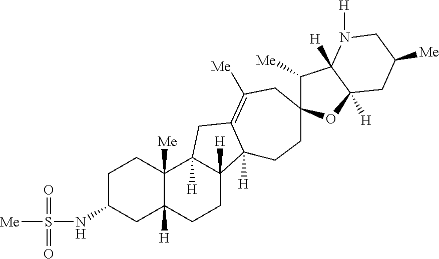
or a pharmaceutically acceptable salt thereof.

20. A kit for evaluating a sample from a cancer patient, to detect a hedgehog-associated biomarker chosen from one or more of: nuclear Gli1, a cilium marker, a marker of a hedgehog pathway, a genomic marker, a marker of Epithelial to Mesenchymal Transition (EMT), a Gemcitabine marker, or an alteration in tumor architecture, said kit comprising a reagent that specifically detects the hedgehog-associated biomarker, and instructions for use.
